(19) United States
(12) Patent Application Publication
(10) Pub. No.: US 2026/0211323 A1
(43) Pub. Date: Jul. 23, 2026

(54) RADIATION-SENSITIVE COMPOSITION, METHOD FOR FORMING PATTERN, AND ONIUM SALT

(71) Applicant: JSR CORPORATION, Tokyo (JP)

(72) Inventors: Ryuichi NEMOTO, Tokyo (JP); Kensuke MIYAO, Tokyo (JP); Satoshi OKAZAKI, Tokyo (JP); Hajime INAMI, Tokyo (JP); Taku OSHIRO, Tokyo (JP)

(73) Assignee: JSR CORPORATION, Tokyo (JP)

(21) Appl. No.: 19/569,093

(22) Filed: Mar. 17, 2026

Related U.S. Application Data
(63) Continuation-in-part of application No. PCT/JP2024/032894, filed on Sep. 13, 2024.

(30) Foreign Application Priority Data
Sep. 26, 2023 (JP) 2023-163070

(51) Int. Cl. G03F 7/004; C07C 309/17; C07C 381/12; C07D 317/70; C08F 20/10; G03F 7/00; G03F 7/038; G03F 7/039; G03F 7/20

(52) U.S. Cl. CPC G03F 7/0045; C07C 309/17; C07C 381/12; C07D 317/70; C08F 20/10; G03F 7/0382; G03F 7/0388; G03F 7/0397; G03F 7/2002; G03F 7/70008

(57) ABSTRACT

A radiation-sensitive composition includes: an onium salt compound represented by formula (1); a polymer including a structural unit (I) represented by formula (2); and a solvent. A is a (1+n)-valent organic group having 3 to 40 carbon atoms including a cyclic structure, when the group represented by A includes a linear alkanediyl group, a carbon number of the linear alkanediyl group is 1 or 2, the group represented by A does not contain a cyclohexylcarbonyloxy structure, R^{f1} and R^{f2} are each independently a hydrogen atom, a monovalent organic group having 1 to 20 carbon atoms, a fluorine atom, or a monovalent fluorinated hydrocarbon group, at least one of R^{f1} and R^{f2} is a fluorine atom or a monovalent fluorinated hydrocarbon group. W is a monocyclic lactone structure, a monocyclic carbonate structure, a monocyclic sultone structure, a monocyclic sulfone structure, or a monocyclic ether structure.

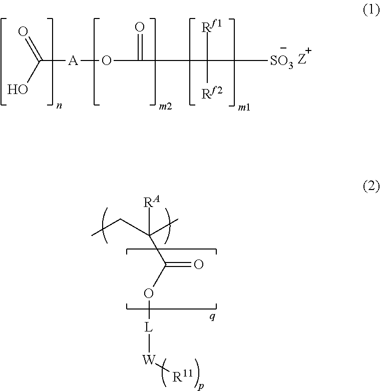

### CROSS-REFERENCE TO RELATED APPLICATIONS

The present application is a continuation-in-part application of International Patent Application No. PCT/JP2024/032894 filed Sep. 13, 2024, which claims priority to Japanese Patent Application No. 2023-163070 filed Sep. 26, 2023. The contents of these applications are incorporated herein by reference in their entirety.

### BACKGROUND OF THE DISCLOSURE

### Technical Field

The present disclosure relates to a radiation-sensitive composition, a method for forming a pattern, and an onium salt.

### Background Art

A photolithography technology using a resist composition has been used for the fine circuit formation in a semiconductor device. As the representative procedure, for example, a resist pattern is formed on a substrate by generating an acid by irradiating the coating of the resist composition with a radioactive ray through a mask pattern, and then reacting in the presence of the acid as a catalyst to generate the difference of solubility of a polymer into a developer such as an alkaline or organic solvent between an exposed part and a non-exposed part.

In the photolithography technique, pattern miniaturization is promoted by using short-wavelength radiation such as ArF excimer laser, or by combining such radiation with an immersion exposure method (liquid immersion lithography). As a next-generation technology, lithography using shorter wavelength radiation such as electron beams, X-rays and EUV (extreme ultraviolet rays) is also being studied.

On a photoacid generator, which is a main component of a resist composition, an acid generator in which a specific functional group is introduced into an anion has been studied from the viewpoint of pattern roughness (see WO 2021/199841 A1).

### SUMMARY

According to an aspect of the present disclosure, a radiation-sensitive composition includes: an onium salt compound represented by formula (1); a polymer including a structural unit (I) represented by formula (2); and a solvent.

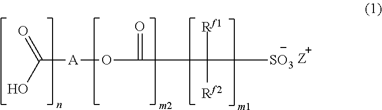

In the formula (1), A is a (1+n)-valent organic group having 3 to 40 carbon atoms including a cyclic structure, when the group represented by A includes a linear alkanediyl group, a carbon number of the linear alkanediyl group is 1 or 2, the group represented by A does not contain a cyclohexylcarbonyloxy structure, R^{f1} and R^{f2} are each independently a hydrogen atom, a monovalent organic group having 1 to 20 carbon atoms, a fluorine atom, or a monovalent fluorinated hydrocarbon group, when there are a plurality of R^{f1}s and R^{f2}s, the plurality of R^{f1}s and R^{f2}s are the same or different from each other, at least one of R^{f1} and R^{f2} is a fluorine atom or a monovalent fluorinated hydrocarbon group, m1 is an integer of 1 to 4, m2 is 0 or 1, n is an integer of 1 to 3, Z⁺ is a monovalent radiation-sensitive onium cation, and the number of fluorine atoms in Z⁺ is 8 or less.

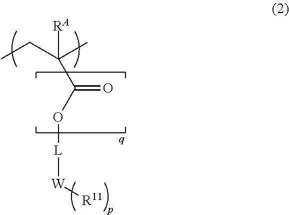

In the formula (2), R^{A} is a hydrogen atom, a fluorine atom, a methyl group, or a trifluoromethyl group, L is a single bond or a divalent linking group, W is a monocyclic lactone structure, a monocyclic carbonate structure, a monocyclic sultone structure, a monocyclic sulfone structure, or a monocyclic ether structure, R¹¹ is a monovalent organic group having 1 to 10 carbon atoms, a cyano group, a nitro group, a hydroxy group, or an amino group, when there are a plurality of R¹¹s, the plurality of R¹¹s are each the same or different from each other, p is an integer of 0 to 3, and q is 0 or 1.

According to another aspect of the present disclosure, a method for forming a pattern includes: applying the above-described radiation-sensitive composition directly or indirectly to a substrate to form a resist film; exposing the resist film to light; and developing the exposed resist film with a developer.

According to a further aspect of the present disclosure, an onium salt compound is represented by formula (1-d).

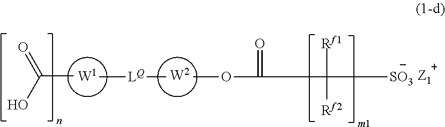

In the formula (1-d), W¹ and W² are each independently a substituted or unsubstituted cyclic structure, L^{Q} is a single bond, *—OCO—, or *—O—CO—O—, * is a bond on the W¹ side, R^{f1} and R^{f2} are each independently a hydrogen atom, a monovalent organic group having 1 to 20 carbon atoms, a fluorine atom, or a monovalent fluorinated hydrocarbon group, when there are a plurality of R^{f1}s and R^{f2}s, the plurality of R^{f1}s and R^{f2}s are the same or different from each other, at least one of R^{f1} and R^{f2} is a fluorine atom or a monovalent fluorinated hydrocarbon group, ml is an integer of 1 to 4, n is an integer of 1 to 3, and Z₁⁺ represents a monovalent radiation-sensitive onium cation.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

Among efforts for next-generation technologies, the resist composition is required to have various resist performances equal to or higher than conventional ones in terms of sensitivity, critical dimension uniformity (CDU) performance that is an index of uniformity of line width and hole diameter, pattern circularity indicating roundness of a hole shape, development defect suppression performance, depth of focus, line width roughness (LWR) performance indicating variation in line width of a resist pattern, pattern rectangularity indicating rectangularity of a cross-sectional shape of a resist pattern, exposure latitude (EL), and the like. However, existing radiation-sensitive compositions do not provide sufficient levels of these properties.

Since the radiation-sensitive composition of the present disclosure contains an onium salt compound as a radiation-sensitive acid generator and a polymer containing a specific structural unit (I) in combination, the radiation-sensitive composition can exhibit sensitivity, CDU performance, pattern circularity, development defect suppression performance, depth of focus, LWR performance, pattern rectangularity, and exposure latitude at a sufficient level when a pattern is formed. Although not bound by any theory, the reason for this can be presumed as follows.

Since the anion of the onium salt compound has a carboxy group, solubility in an alkaline developer is higher than that of a conventional onium salt compound. In addition, the interaction between the carboxy group and other components such as a polymer in the radiation-sensitive composition is enhanced, and steric hindrance occurs due to inclusion of a cyclic structure in the anion, whereby the diffusion length of the generated acid is shortened. In addition, by setting the number of fluorine atoms in the cation of the onium salt compound to a predetermined number, it is possible to achieve a balance among acid generation efficiency, solubility in a developer, and dispersibility.

By these actions of the onium salt compound, occurrence of roughness such as variations in pattern shape and unevenness and development defects can be suppressed. In addition, since the polymer contains the structural unit (I) into which a highly polar monocyclic structure is introduced, solubility in an alkaline developer or dissolution inhibition in an organic solvent developer is high. In addition, by adopting a polar monocyclic structure, the bulkiness is suppressed, the polymer easily approaches another polymer or the onium salt compound, and the interaction ability between the components is improved. By the synergistic effect of the above, the diffusion length of the generated acid can be specifically shortened, and the occurrence of roughness such as variations in pattern shape and unevenness can be remarkably suppressed. When the group A has a cyclohexylcarbonyloxy structure (that is, when the anion contains a carboxycyclohexylcarbonyloxy structure), the carboxylic acid moiety nucleophilically attacks a nearby carbonyl group to form an anhydride ring, so that storage stability may deteriorate and roughness may deteriorate. By using the respective properties of the onium salt compound and the polymer as described above in combination, it is presumed that given various resist performances can be exhibited. The organic group refers to a group containing at least one carbon atom.

In the method for forming a pattern of the present disclosure, since the radiation-sensitive composition capable of exhibiting sensitivity, CDU performance, pattern circularity, development defect suppression performance, depth of focus, LWR performance, pattern rectangularity, and exposure latitude at a sufficient level is used when a pattern is formed, a high-quality resist pattern can be efficiently formed.

The onium salt compound of the present disclosure has a specific structure including a cyclic structure to which a carboxy group is bonded, and may have moderate steric hindrance, rigidity, hydrophilicity, interactivity with other components, and the like, and thus is suitable as a radiation-sensitive acid generator of the radiation-sensitive composition.

Hereinbelow, embodiments of the present disclosure will be described in detail, but the present disclosure is not limited to these embodiments. Combinations of suitable embodiments are also preferable.

### <Radiation-Sensitive Composition>

The radiation-sensitive composition (hereinafter also simply referred to as “composition”) according to the present embodiment includes an onium salt compound, a polymer containing a structural unit (I) (hereinafter, also referred to as a “base polymer”), and a solvent. The composition may contain other optional components as long as the effects of the present disclosure are not impaired.

### (Onium Salt Compound)

The Onium salt compound is represented by the formula (1), and is a component that generates an acid upon irradiation with radiation. The acid generated by exposure to light has a function of dissociating the acid-dissociable group of the base polymer to generate a carboxy group or the like. The composition may contain one type or two or more types of the onium salt compound.

Examples of a (1+n)-valent organic group having 3 to 40 carbon atoms and having a cyclic structure represented by A include a group obtained by removing n hydrogen atoms from a monovalent organic group having 3 to 40 carbon atoms and having a cyclic structure. The carboxy group of the above formula (1) is directly bonded to the cyclic structure in A. However, A does not contain a cyclohexylcarbonyloxy structure. The organic group having a cyclic structure is not particularly limited, and examples thereof include a monovalent alicyclic hydrocarbon group having 3 to 20 carbon atoms, a monovalent aromatic hydrocarbon group having 6 to 20 carbon atoms, a heterocyclic group containing a divalent heteroatom-containing linking group between two carbon atoms of the foregoing groups, a group obtained by substituting some or all of the hydrogen atoms of any of the foregoing groups with a monovalent heteroatom-containing substituent, and a combination thereof. When a plurality of rings are combined, two adjacent rings may form any of a fused ring, a spiro ring, and a ring assembly (a structure in which adjacent rings are bonded by a single bond), and may be linked in a chain structure. Examples of the chain structure include a single bond, an alkanediyl group having 1 or 2 carbon atoms, a branched alkanediyl group having 3 to 10 carbon atoms, a divalent heteroatom-containing linking group, and a group obtained by combining these groups. Examples of the divalent heteroatom-containing linking group include —CO—, —CS—, —NH—, —O—, —S—, —SO—, —SO₂—, and a group obtained by combining two or more of these.

Examples of the monovalent alicyclic hydrocarbon group having 3 to 20 carbon atoms include cycloalkyl groups such as a cyclopropyl group, a cyclobutyl group, a cyclopentyl group, and a cyclohexyl group; cycloalkenyl groups such as a cyclopropenyl group, a cyclopentenyl group, and a cyclohexenyl group; bridged cyclic saturated hydrocarbon groups such as a norbornyl group, an adamantyl group, a tricyclodecyl group, and a bornyl group; and bridged cyclic unsaturated hydrocarbon groups such as a norbornenyl group and a tricyclodecenyl group.

Examples of the monovalent aromatic hydrocarbon group having 6 to 20 carbon atoms include a phenyl group, a tolyl group, a naphthyl group, an anthracenyl group, a pyrenyl group, and a fluorenyl group.

Examples of the heteroatom that constitutes the divalent heteroatom-containing linking group or the monovalent heteroatom-containing substituent include an oxygen atom, a nitrogen atom, a sulfur atom, a phosphorus atom, a silicon atom, and a halogen atom. Examples of the halogen atom include a fluorine atom, a chlorine atom, a bromine atom, and an iodine atom.

As the divalent heteroatom-containing linking group, the divalent heteroatom-containing linking group can be suitably employed.

Examples of the monovalent heteroatom-containing substituent include a hydroxy group, a sulfanyl group, a cyano group, a nitro group, and a halogen atom.

Examples of the heterocyclic group include a group obtained by removing one hydrogen atom from an aromatic heterocyclic structure and a group obtained by removing one hydrogen atom from an aliphatic heterocyclic structure. A 5-membered aromatic structure having aromaticity due to introduction of a hetero atom is also included in the heterocyclic structure. Examples of the hetero atom include an oxygen atom, a nitrogen atom, and a sulfur atom.

Examples of the aromatic heterocyclic structure include:
oxygen atom-containing aromatic heterocyclic structures such as furan, pyran, benzofuran, and benzopyran;
nitrogen atom-containing aromatic heterocyclic structures such as pyrrole, imidazole, pyridine, pyrimidine, pyrazine, indole, quinoline, isoquinoline, acridine, phenazine, and carbazole;
sulfur atom-containing aromatic heterocyclic structures such as thiophene; and
aromatic heterocyclic structures containing a plurality of hetero atoms such as thiazole, benzothiazole, thiazine, and oxazine.

Examples of the aliphatic heterocyclic structure include:
oxygen atom-containing aliphatic heterocyclic structures such as oxirane, tetrahydrofuran, tetrahydropyran, dioxolane, and dioxane;
nitrogen atom-containing aliphatic heterocyclic structures such as aziridine, pyrrolidine, piperidine, and piperazine;
sulfur atom-containing aliphatic heterocyclic structures such as thietane, thiolane, and thiane; and
aliphatic heterocyclic structures containing a plurality of hetero atoms such as morpholine, 1,2-oxathiolane, and 1,3-oxathiolane.

Examples of the cyclic structure corresponding to the heterocyclic group also include a lactone structure, a cyclic carbonate structure, a sultone structure, and a structure containing a cyclic acetal. Examples of such structures include structures represented by formulas (H-1) to (H-11) below.

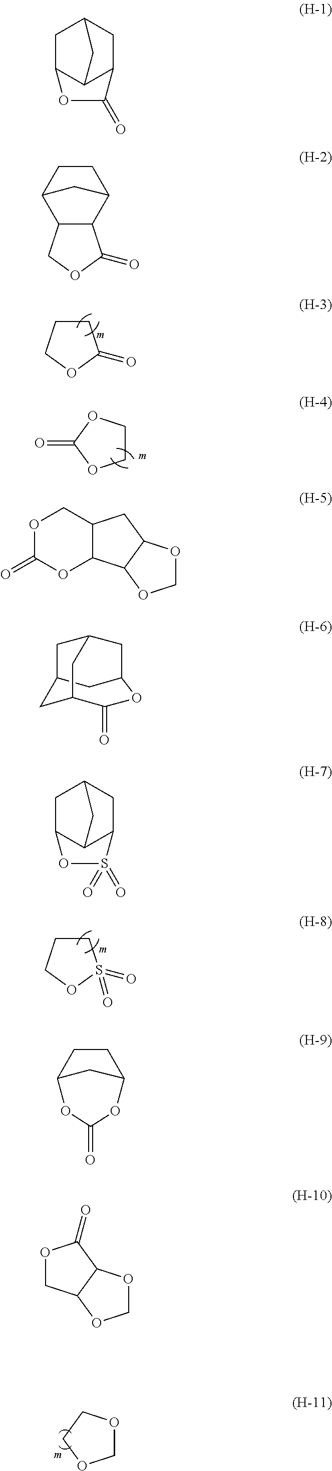

In the formulas, m's are each an integer of 1 to 3.

In the formula (1), the cyclic structure is preferably an alicyclic hydrocarbon structure, an aromatic hydrocarbon structure, a lactone structure, a sultone structure, a cyclic acetal structure, a cyclic carbonate structure, a cyclic ether structure, or a combination thereof. Examples of the alicyclic hydrocarbon structure include alicyclic monocyclic structures such as a cyclopentane structure and a cyclohexane structure, and alicyclic polycyclic structures such as an adamantane structure and a norbornane structure. Among them, an alicyclic polycyclic structure is preferable as the alicyclic hydrocarbon structure. As the aromatic hydrocarbon structure, a structure corresponding to the monovalent aromatic hydrocarbon group having 6 to 20 carbon atoms can be suitably employed. When the cyclic organic group has one or two or more of these structures, the diffusion length of the acid generated by exposure can be appropriately controlled, and the composition can exhibit the various resist performances at a high level.

Examples of the monovalent fluorinated hydrocarbon groups represented by R^{f1} and R^{f2} include a monovalent fluorinated chain hydrocarbon group having 1 to 20 carbon atoms and a monovalent fluorinated alicyclic hydrocarbon group having 3 to 20 carbon atoms.

Examples of the monovalent fluorinated chain hydrocarbon group having 1 to 20 carbon atoms include:
fluorinated alkyl groups such as a trifluoromethyl group, a difluoromethyl group, a 2,2,2-trifluoroethyl group, a pentafluoroethyl group, a 2,2,3,3,3-pentafluoropropyl group, a 1,1,1,3,3,3-hexafluoropropyl group, a heptafluoro-n-propyl group, a heptafluoro-i-propyl group, a nonafluoro-n-butyl group, a nonafluoro-i-butyl group, a nonafluoro-t-butyl group, a 2,2,3,3,4,4,5,5-octafluoro-n-pentyl group, a tridecafluoro-n-hexyl group, and a 5,5,5-trifluoro-1,1-diethylpentyl group;
fluorinated alkenyl groups such as a trifluoroethenyl group and a pentafluoropropenyl group; and
fluorinated alkynyl groups such as a fluoroethynyl group and a trifluoropropynyl group.

Examples of the monovalent fluorinated alicyclic hydrocarbon group having 3 to 20 carbon atoms include:
fluorinated cycloalkyl groups such as a fluorocyclopentyl group, a difluorocyclopentyl group, a nonafluorocyclopentyl group, a fluorocyclohexyl group, a difluorocyclohexyl group, an undecafluorocyclohexylmethyl group, a fluoronorbornyl group, a fluoroadamantyl group, a fluorobornyl group, a fluoroisobornyl group, and a fluorotricyclodecyl group; and
fluorinated cycloalkenyl groups such as a fluorocyclopentenyl group and a nonafluorocyclohexenyl group.

As the fluorinated hydrocarbon group, a monovalent fluorinated chain hydrocarbon group having 1 to 8 carbon atoms is preferable, and a monovalent fluorinated linear hydrocarbon group having 1 to 5 carbon atoms is more preferable.

R^{f1} and R^{f2} are preferably each independently a fluorine atom or a trifluoromethyl group. R^{f1} and R^{f2} on the carbon atom bonded to —SO₃⁻ in the formula (1) are preferably fluorine atoms.

m1 is preferably an integer of 1 to 3, and more preferably 1 or 2.

n is preferably 1 or 2, and more preferably 1.

The onium salt compound is preferably a compound represented by any one of the following formulas (1-a) to (1-e). The carboxy group itself has a certain degree of freedom while increasing the rigidity of the anion structure, and thus exhibits high interaction ability with the monocyclic lactone moiety of the polymer. As a result, the generated acid diffusion length can be controlled more precisely, and various performances such as CDU, LWR, and exposure latitude can be improved.

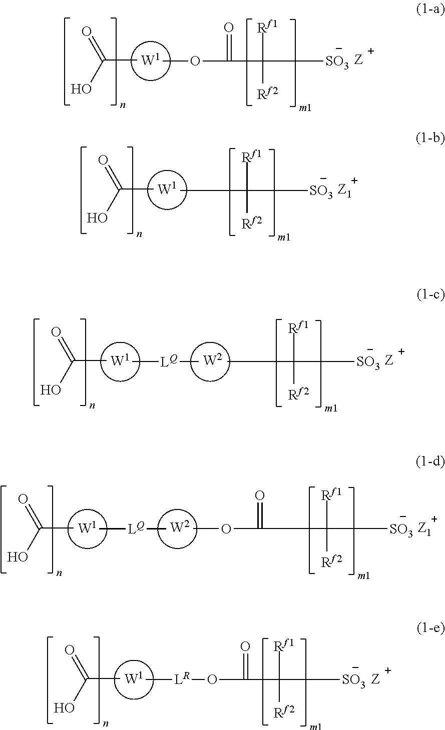

In the formulas (1-a) to (1-e),
W¹ and W² are each independently a substituted or unsubstituted cyclic structure,
L^{P} is a single bond or a divalent linking group,
L^{Q} is a single bond, *—OCO—, or *—O—CO—O—, * is a bond on a W¹ side,
L^{R} is a group obtained by substituting one or both of a methylene group and a hydrogen atom of a methylene group with a hydrocarbon group having 1 to 10 carbon atoms, or *—OCO—R″—, R″ is a group obtained by substituting one or both of a methylene group and a hydrogen atom of a methylene group with a hydrocarbon group having 1 to 10 carbon atoms, * is a bond on the W¹ side,
Z₁⁺ is a monovalent radiation-sensitive onium cation, and
R^{f1}, R^{f2}, Z⁺, m1, and n have the same meanings as in the above formula (1).

As the cyclic structure in W¹ and W², the cyclic structures represented as A in the formula (1) can suitably be employed. When W¹ and W² have substituents, examples of the substituent include a halogen atom such as a fluorine atom, a chlorine atom, a bromine atom, and an iodine atom; a hydroxy group; a carboxy group; a cyano group; a nitro group; an alkyl group, an alkoxy group, an alkoxycarbonyl group, an alkoxycarbonyloxy group, an acyl group, an acyloxy group, an aryloxy group, or groups obtained by substituting a hydrogen atom on these groups with a halogen atom; and an oxo group (═O).

As the divalent linking group represented by L^{P}, the chain structures represented as A in the formula (1) can suitably be employed.

Examples of the hydrocarbon group having 1 to 10 carbon atoms in L^{R} include a monovalent chain hydrocarbon group having 1 to 10 carbon atoms, a monovalent alicyclic hydrocarbon group having 3 to 5 carbon atoms, and a monovalent aromatic hydrocarbon group having 6 to 10 carbon atoms.

Among them, the onium salt compound is preferably a compound represented by the above formula (1-a), (1-b), (1-c), or (1-d), particularly preferably a compound represented by the above formula (1-b), (1-c), or (1-d).

Specific examples of the anion moiety of the onium salt compound include, but are not limited to, the structures represented by the following formulas (1-1-1) to (1-1-93).

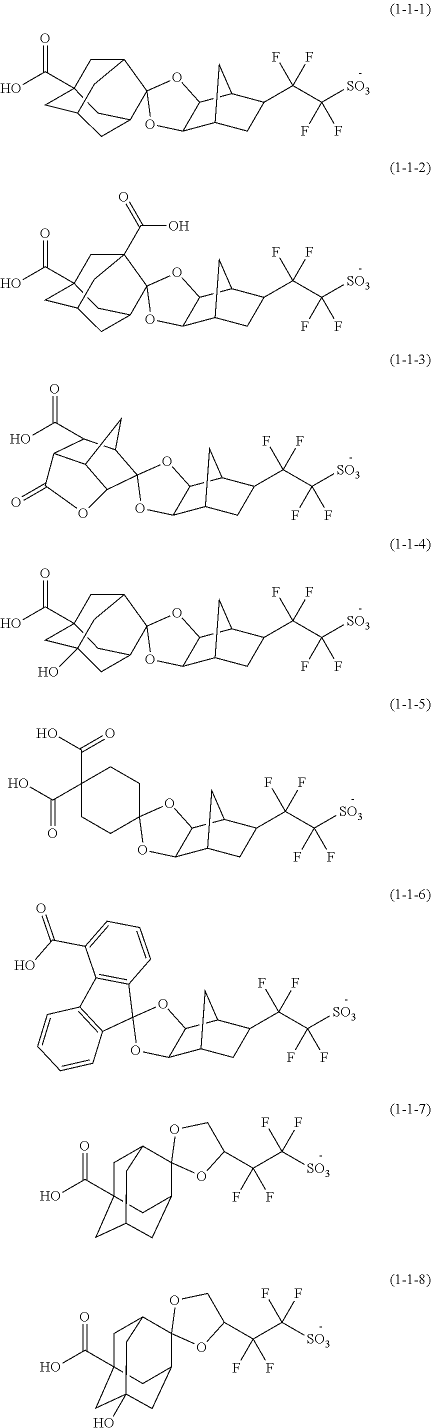

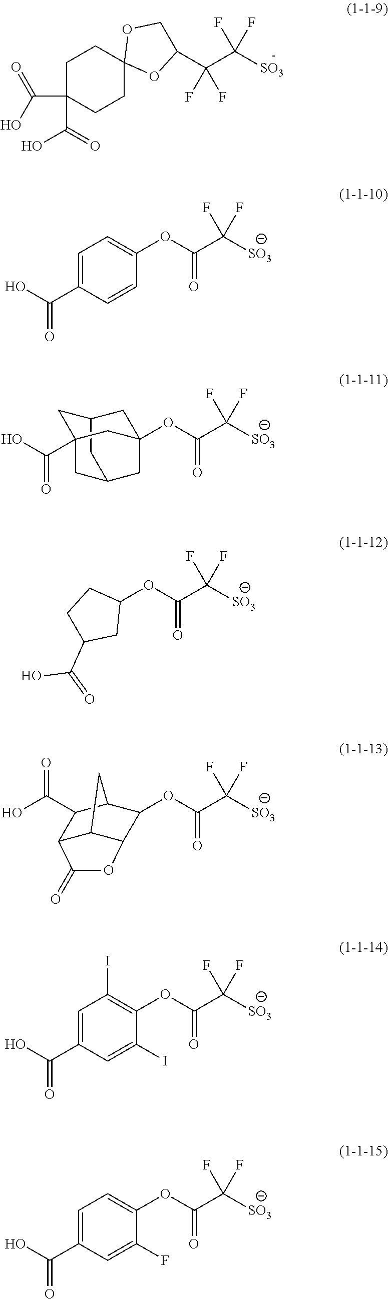

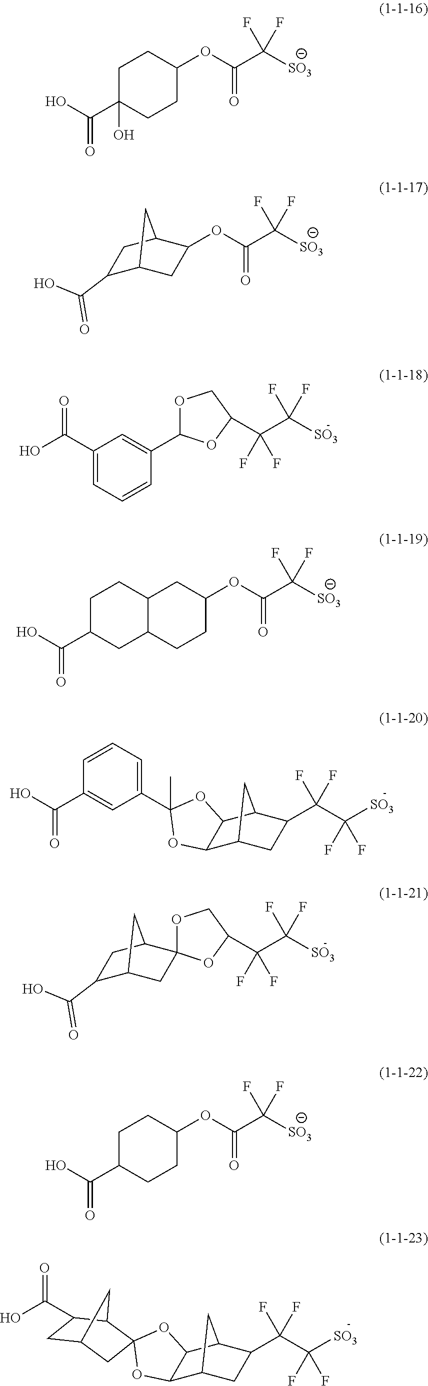

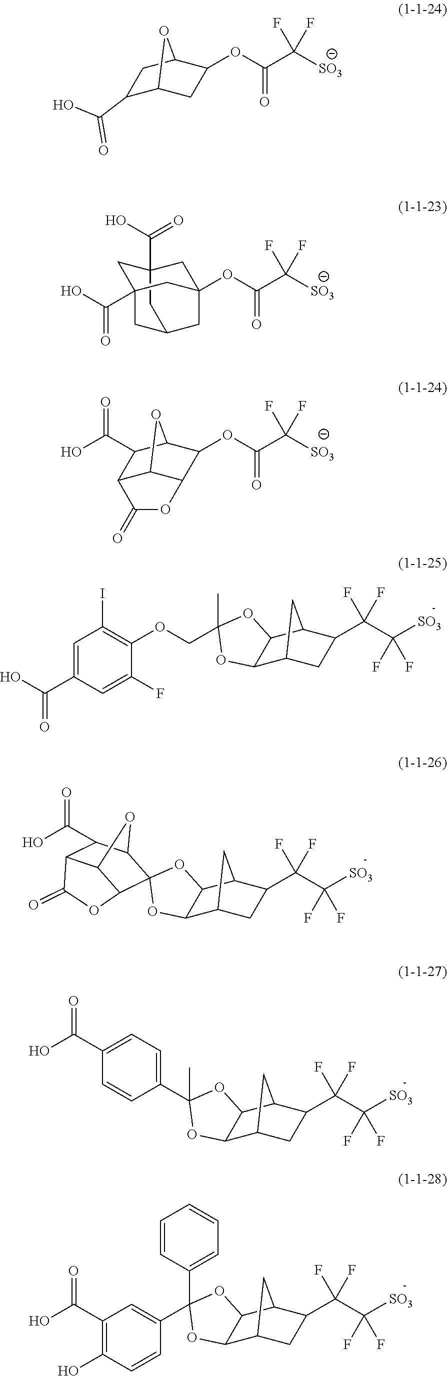

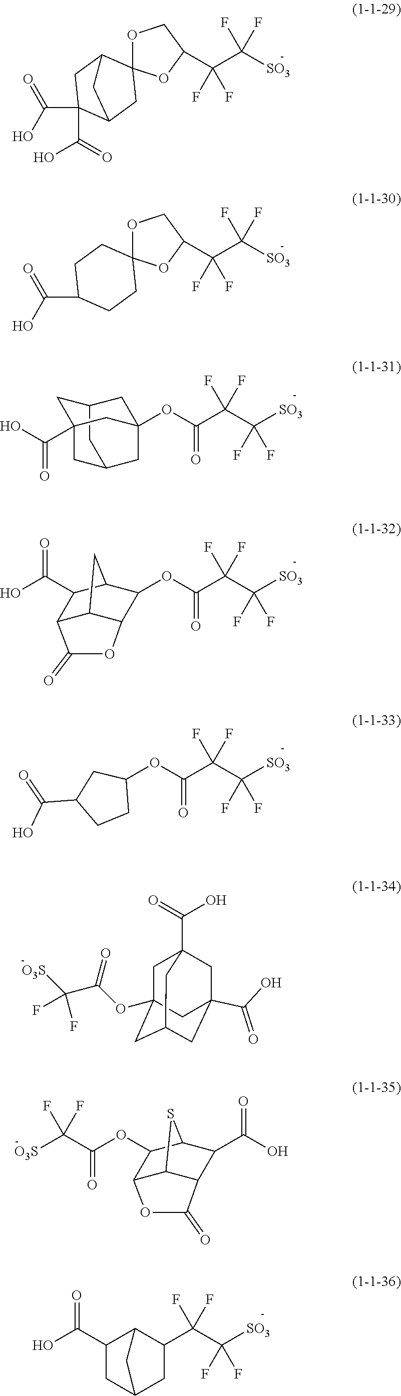

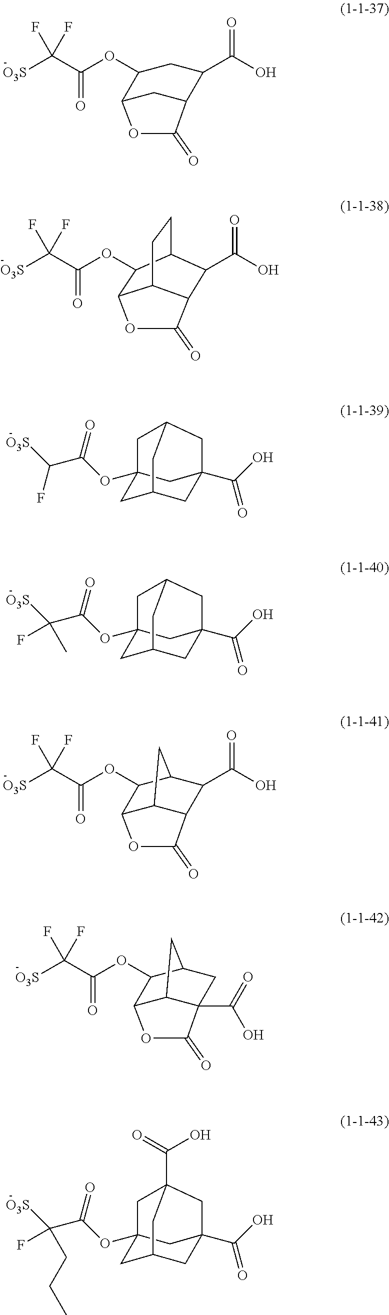

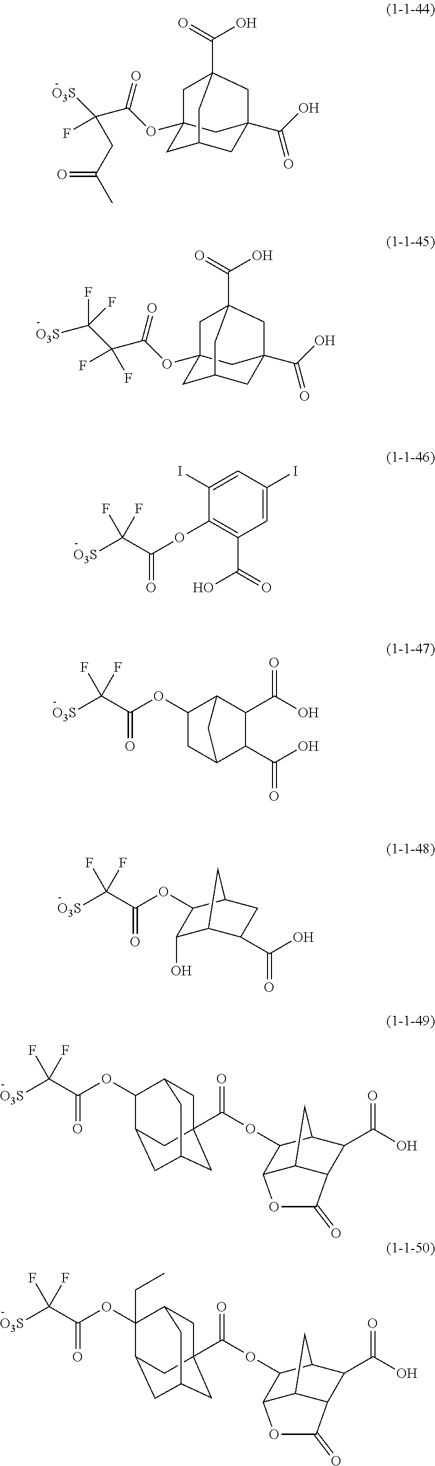

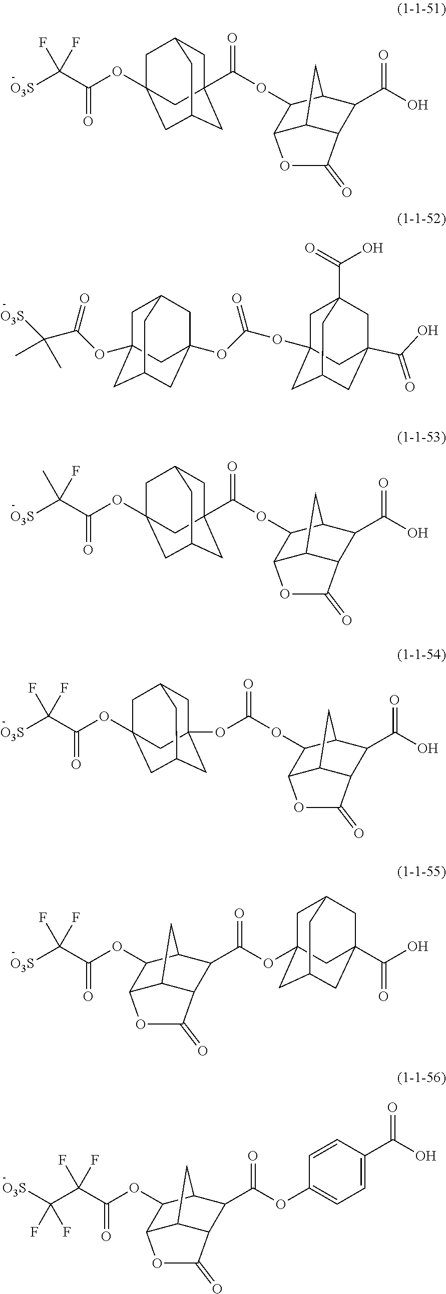

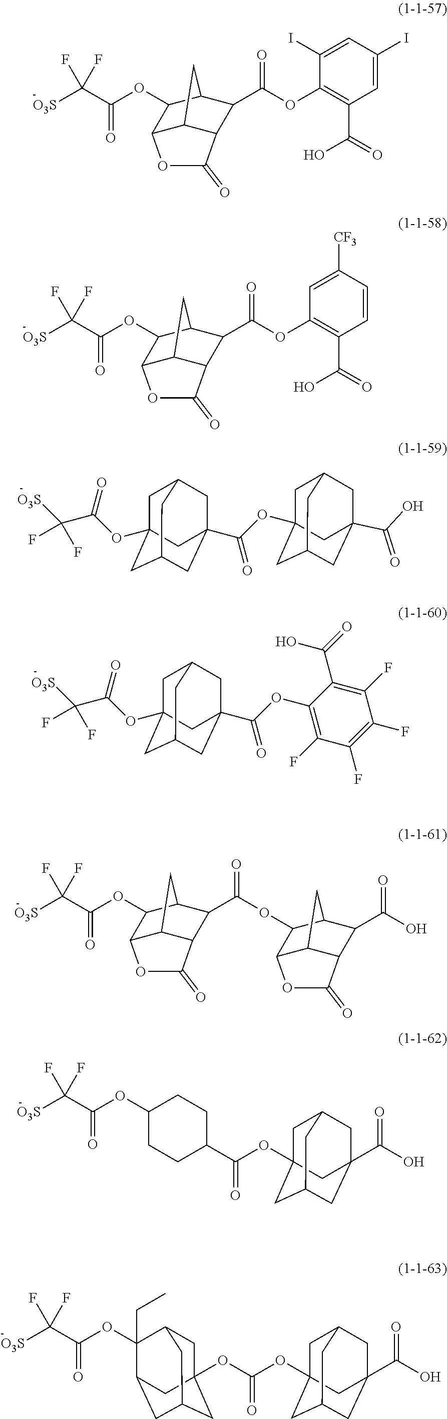

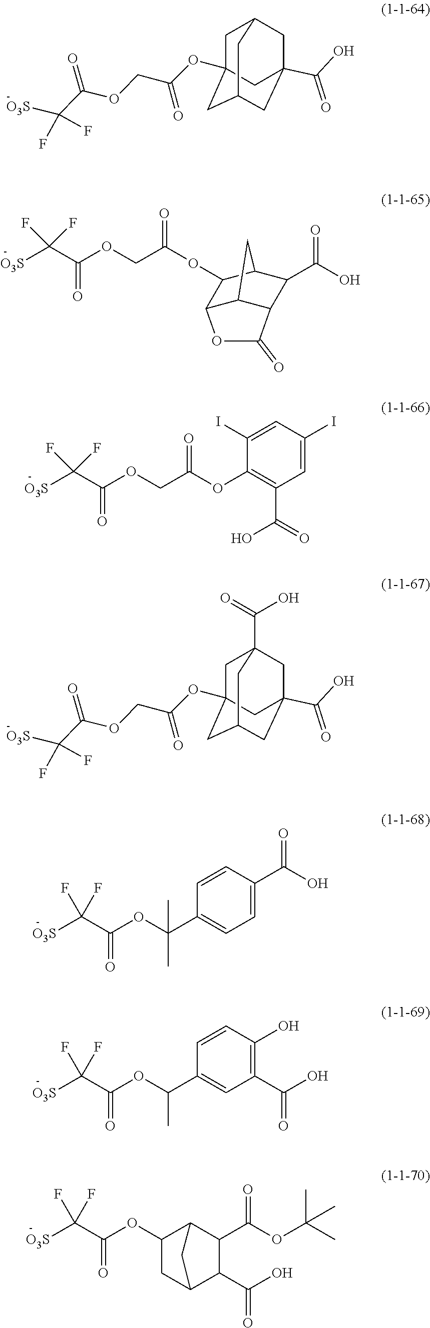

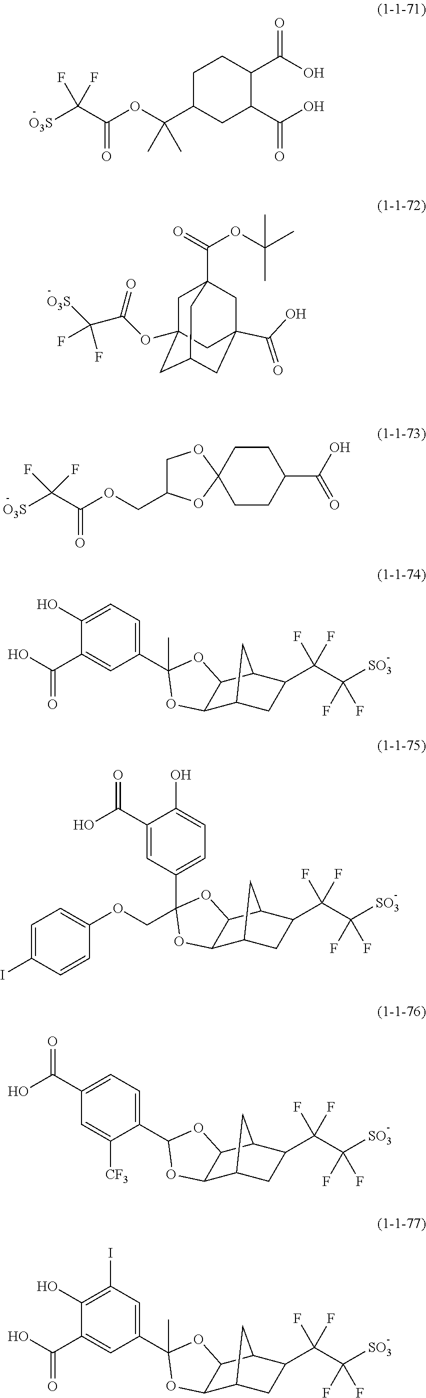

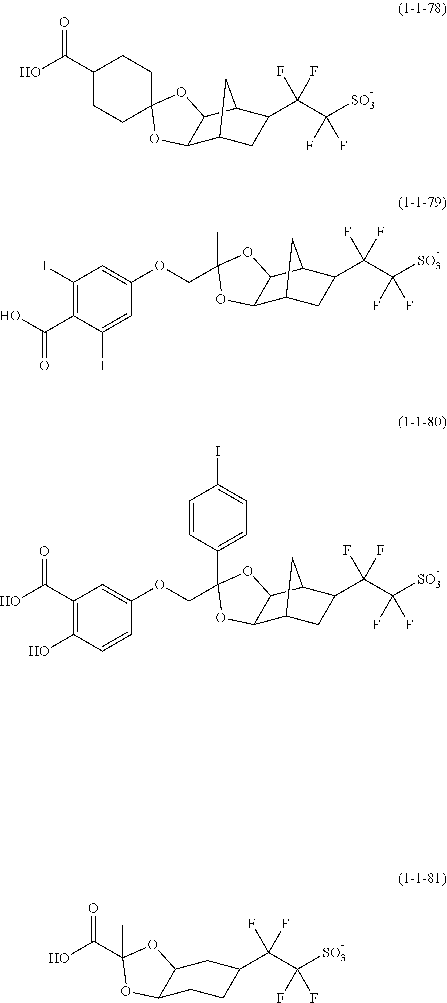

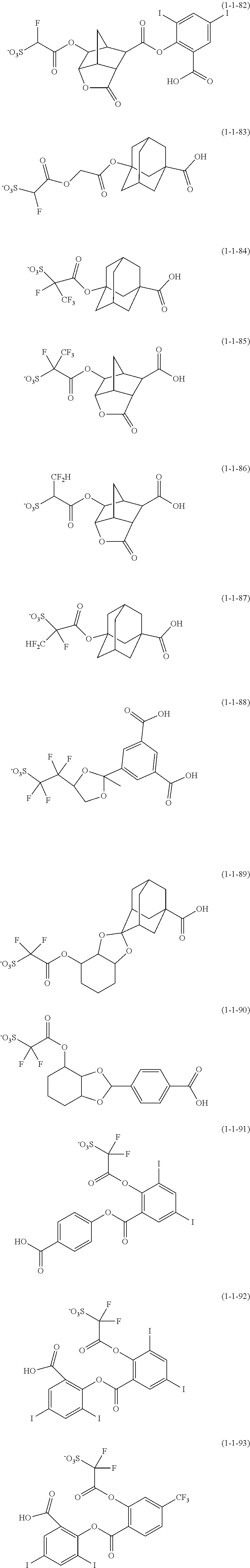

An example of the monovalent radiation-sensitive onium cation represented by Z⁺ and Z₁⁺ of the formula (1) is a radioactive ray-degradable onium cation containing an element such as S, I, O, N, P, Cl, Br, F, As, Se, Sn, Sb, Te, or Bi. Examples of such a radioactive ray-degradable onium cation include a sulfonium cation, a tetrahydrothiophenium cation, an iodonium cation, a phosphonium cation, a diazonium cation, and a pyridinium cation. Among them, a sulfonium cation or an iodonium cation is preferred, and a sulfonium cation is more preferred.

The number of fluorine atoms in Z⁺ is 8 or less. As a result, the dissolution promoting effect during alkali development and the dissolution inhibition effect during organic solvent development are both remarkably exhibited. In addition, the acid generation efficiency and the dispersibility can be balanced without impairing the polarity of the anion, and the pattern roughness and the shape improving effect can be exhibited.

The sulfonium cation or iodonium cation is preferably represented by the following formulas (X-1) to (X-6).

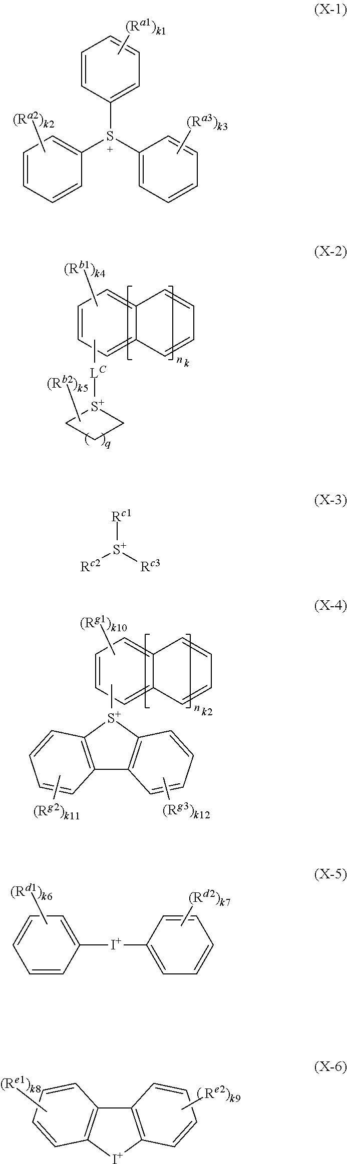

In the formula (X-1), R^{a1}, R^{a2} and R^{a3} are each independently a substituted or unsubstituted, straight or branched chain alkyl group, alkoxy group, alkoxycarbonyloxy group, or (cyclo)alkoxycarbonylalkoxy group having 1 to 12 carbon atoms; a substituted or unsubstituted, monocyclic or polycyclic cycloalkyl group having 3 to 12 carbon atoms; a substituted or unsubstituted aromatic hydrocarbon group having 6 to 12 carbon atoms; a hydroxy group, a halogen atom, —OSO₂—RP, —SO₂—R^{Q}, —S—R^{T}, —O—, —CO— or a combination thereof; or a ring structure obtained by combining two or more of these groups. The ring structure may contain heteroatoms such as 0 and S between the carbon-carbon bonds forming the skeleton. RP, R^{Q} and R^{T} are each independently a substituted or unsubstituted, straight or branched chain alkyl group having 1 to 12 carbon atoms; a substituted or unsubstituted alicyclic hydrocarbon group having 5 to 25 carbon atoms; or a substituted or unsubstituted aromatic hydrocarbon group having 6 to 12 carbon atoms. k1, k2 and k3 are each independently an integer of 0 to 5. When there are a plurality of R^{a1} to R^{a3} and a plurality of R^{P}, R^{Q} and R^{T}, a plurality of R^{a1} to R^{a3} and a plurality of R^{P}, R^{Q} and R^{T} may be each identical or different.

In the formula (X-2), R^{b1} is a substituted or unsubstituted, straight chain or branched alkyl group or alkoxy group, or an alkoxyalkoxy group having 1 to 20 carbon atoms; a substituted or unsubstituted acyl group having 2 to 8 carbon atoms; or a substituted or unsubstituted aromatic hydrocarbon group having 6 to 8 carbon atoms; a hydroxy group; or a halogen atom. nₖ is 0 or 1. When nₖ is 0, k4 is an integer of 0 to 4. When nₖ is 1, k4 is an integer of 0 to 7. When there are a plurality of R^{b1}, a plurality of R^{b1} may be each identical or different. A plurality of R^{b1} may represent a ring structure obtained by combining them. R^{b2} is a substituted or unsubstituted, straight chain or branched alkyl group having 1 to 7 carbon atoms; or a substituted or unsubstituted aromatic hydrocarbon group having 6 or 7 carbon atoms. Lc is a single bond or divalent linking group. k5 is an integer of 0 to 4. When there are a plurality of R^{b2}, a plurality of R^{b2} may be each identical or different. A plurality of R^{b2} may represent a ring structure obtained by combining them. q is an integer of 0 to 3. In the formula, the ring structure containing S⁺ may contain a heteroatom such as 0 or S between the carbon-carbon bonds forming the skeleton.

In the formula (X-3), R^{c1}, R^{c2} and R^{c3} are each independently a substituted or unsubstituted, straight or branched chain alkyl group having 1 to 12 carbon atoms.

In the formula (X-4), R^{g1} is a substituted or unsubstituted linear or branched alkyl or alkoxy group having 1 to 20 carbon atoms, a substituted or unsubstituted acyl group having 2 to 8 carbon atoms, a substituted or unsubstituted aromatic hydrocarbon group having 6 to 8 carbon atoms, or a hydroxy group. nₖ₂ is 0 or 1. When nₖ₂ is 0, k10 is an integer of 0 to 4, and when nₖ₂ is 1, k10 is an integer of 0 to 7. When there are two or more R^{g1}s, the two or more R^{g1}'s are the same or different from each other, and may represent a cyclic structure formed by combining them together. R^{g2} and R^{g3} are each independently a substituted or unsubstituted linear or branched alkyl, alkoxy, or alkoxycarbonyloxy group having 1 to 12 carbon atoms, a substituted or unsubstituted monocyclic or polycyclic cycloalkyl group having 3 to 12 carbon atoms, a substituted or unsubstituted aromatic hydrocarbon group having 6 to 12 carbon atoms, a hydroxyl group, a halogen atom, or a ring structure formed by combining two or more of these groups together. K11 and k12 are each independently an integer of 0 to 4. When there are two or more R^{g2}s and two or more R^{g3}s, the two or more R^{g2}s may be the same or different from each other, and the two or more R^{g3}s may be the same or different from each other.

In the formula (X-5), R^{d1} and R^{d2} are each independently a substituted or unsubstituted, straight or branched chain alkyl group, alkoxy group or alkoxycarbonyl group having 1 to 12 carbon atoms; a substituted or unsubstituted aromatic hydrocarbon group having 6 to 12 carbon atoms; a halogen atom; a halogenated alkyl group having 1 to 4 carbon atoms; a nitro group; or a ring structure obtained by combining two or more of these groups. k6 and k7 are each independently an integer of 0 to 5. When there are a plurality of R^{d1} and a plurality of R^{d2}, a plurality of R^{d1} and a plurality of R^{d2} may be each identical or different.

In the formula (X-6), R^{e1} and R^{e2} are each independently a halogen atom; a substituted or unsubstituted straight or branched chain alkyl group having 1 to 12 carbon atoms; or a substituted or unsubstituted aromatic hydrocarbon group having 6 to 12 carbon atoms. k8 and k9 are each independently an integer of 0 to 4.

Specific examples of the radiation-sensitive onium cation include, but not limited thereto, the structures represented by the formulas (1-2-1) to (1-2-44).

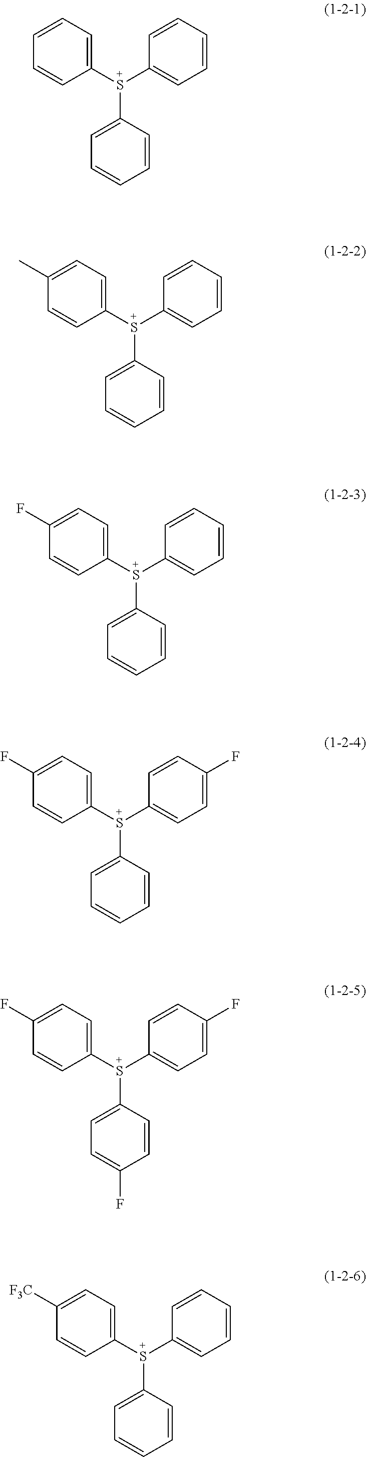

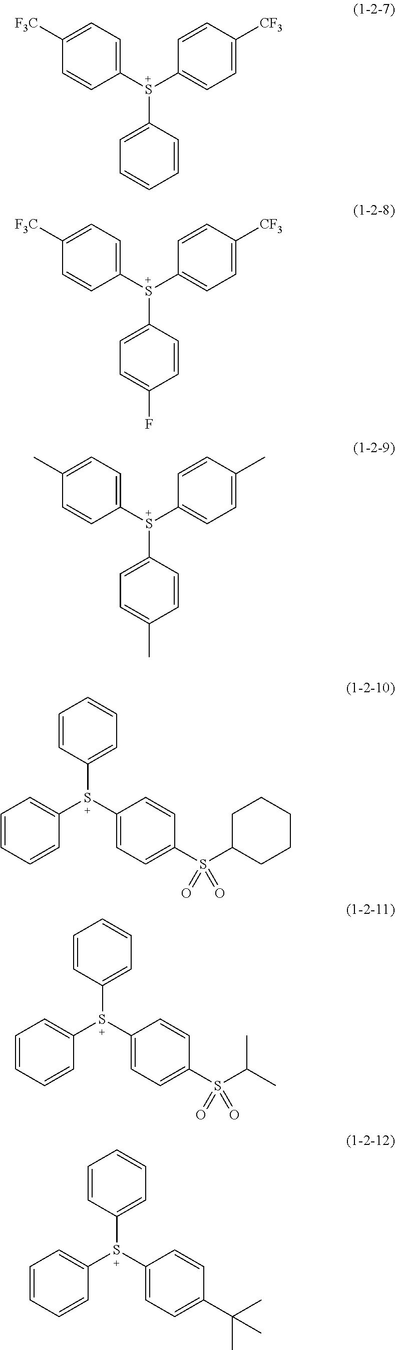

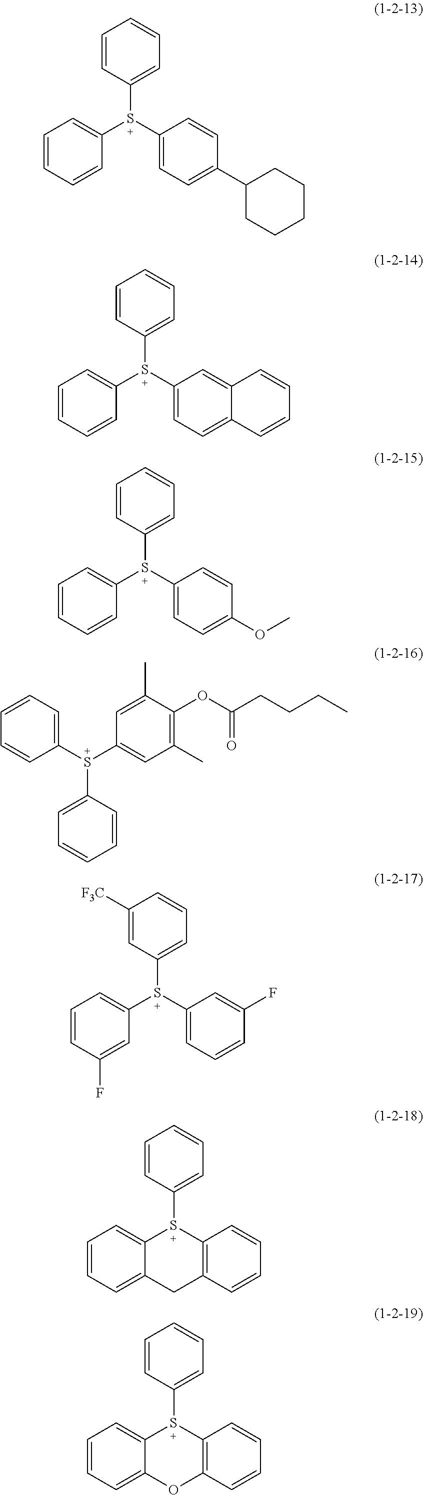

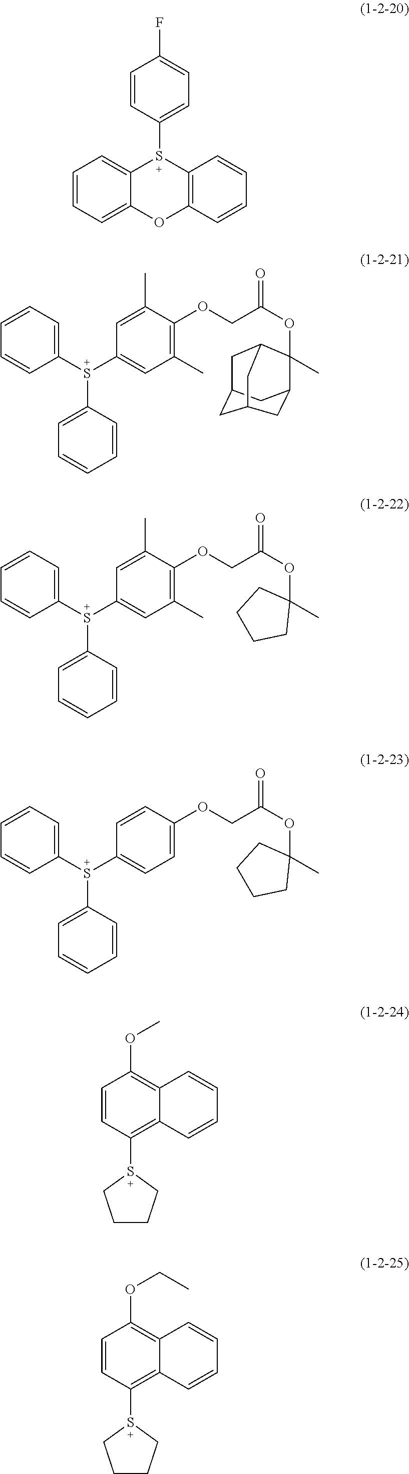

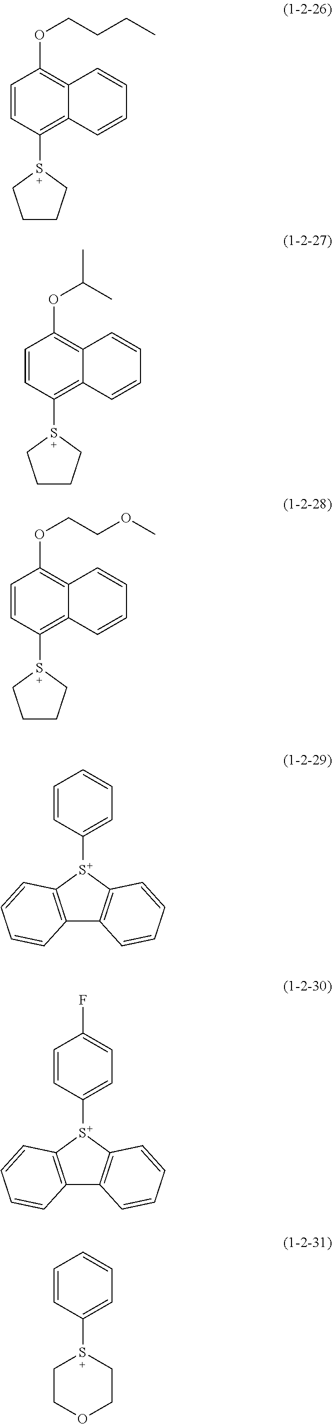

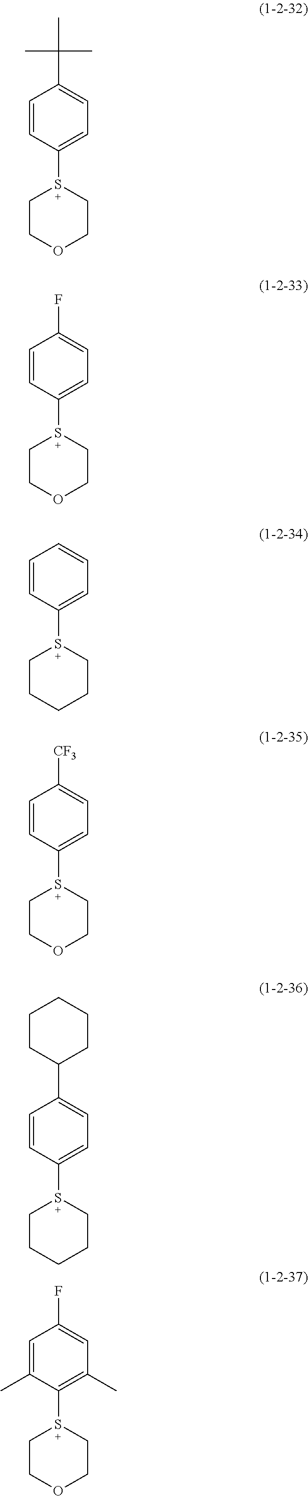

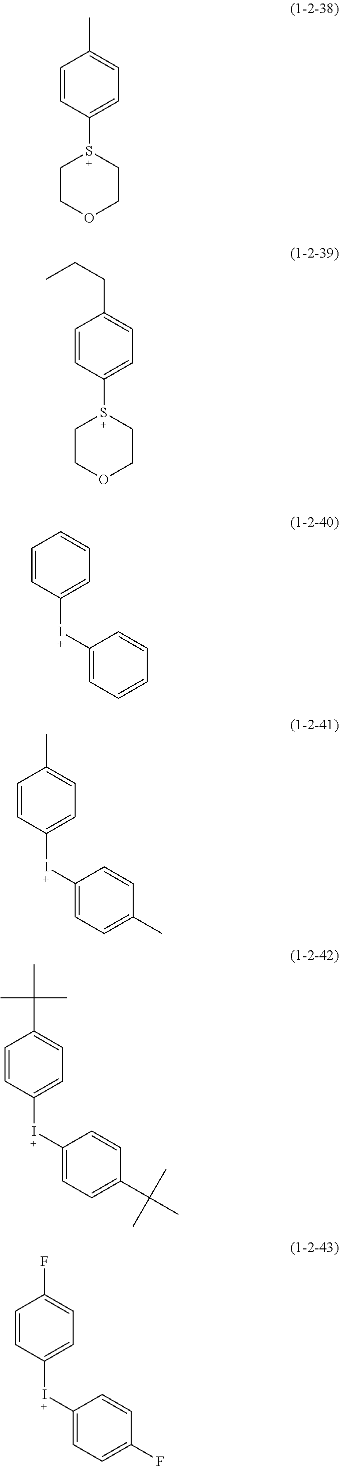

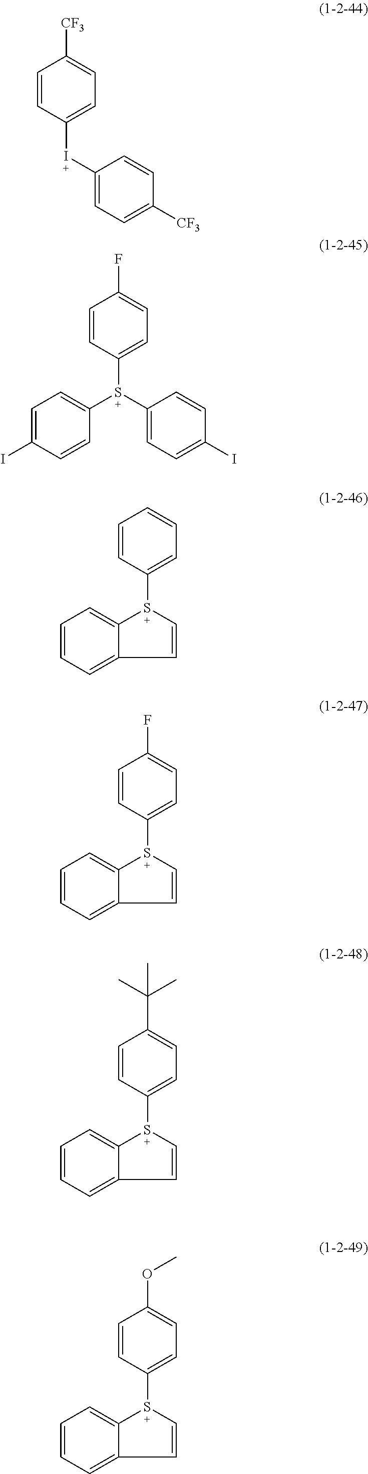

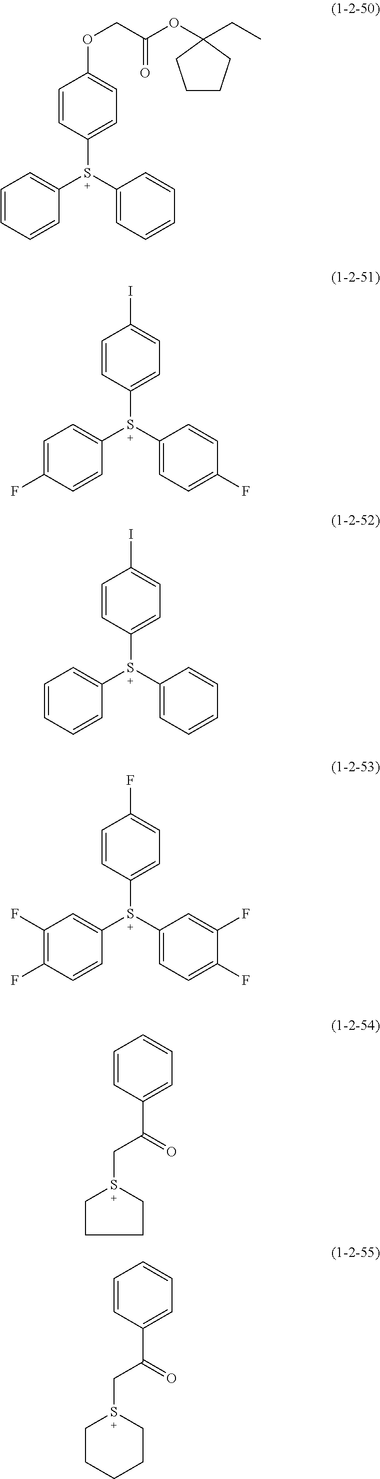

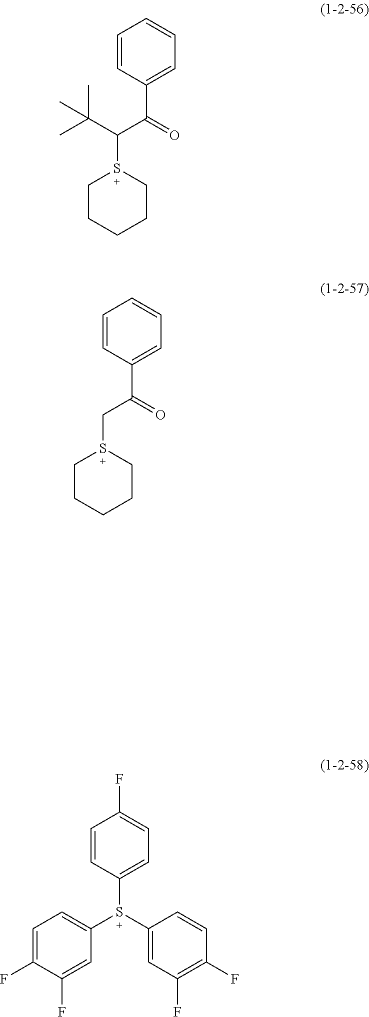

The onium salt compound can be obtained by appropriately combining the above-mentioned anion moiety with the above-mentioned radiation-sensitive onium cation. Specific examples include, but are not limited to, structures of the following formulae (1-1) to (1-55).

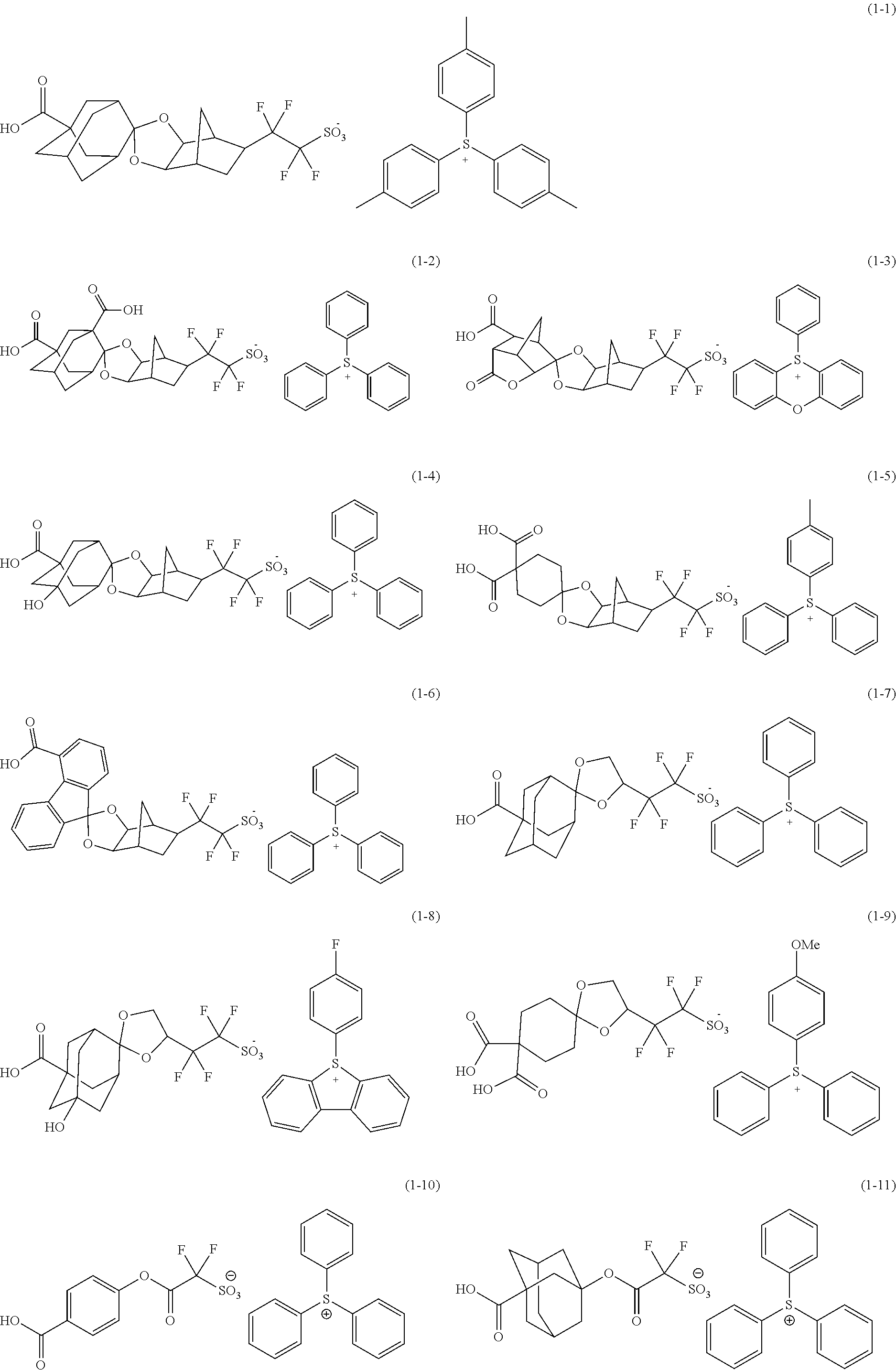

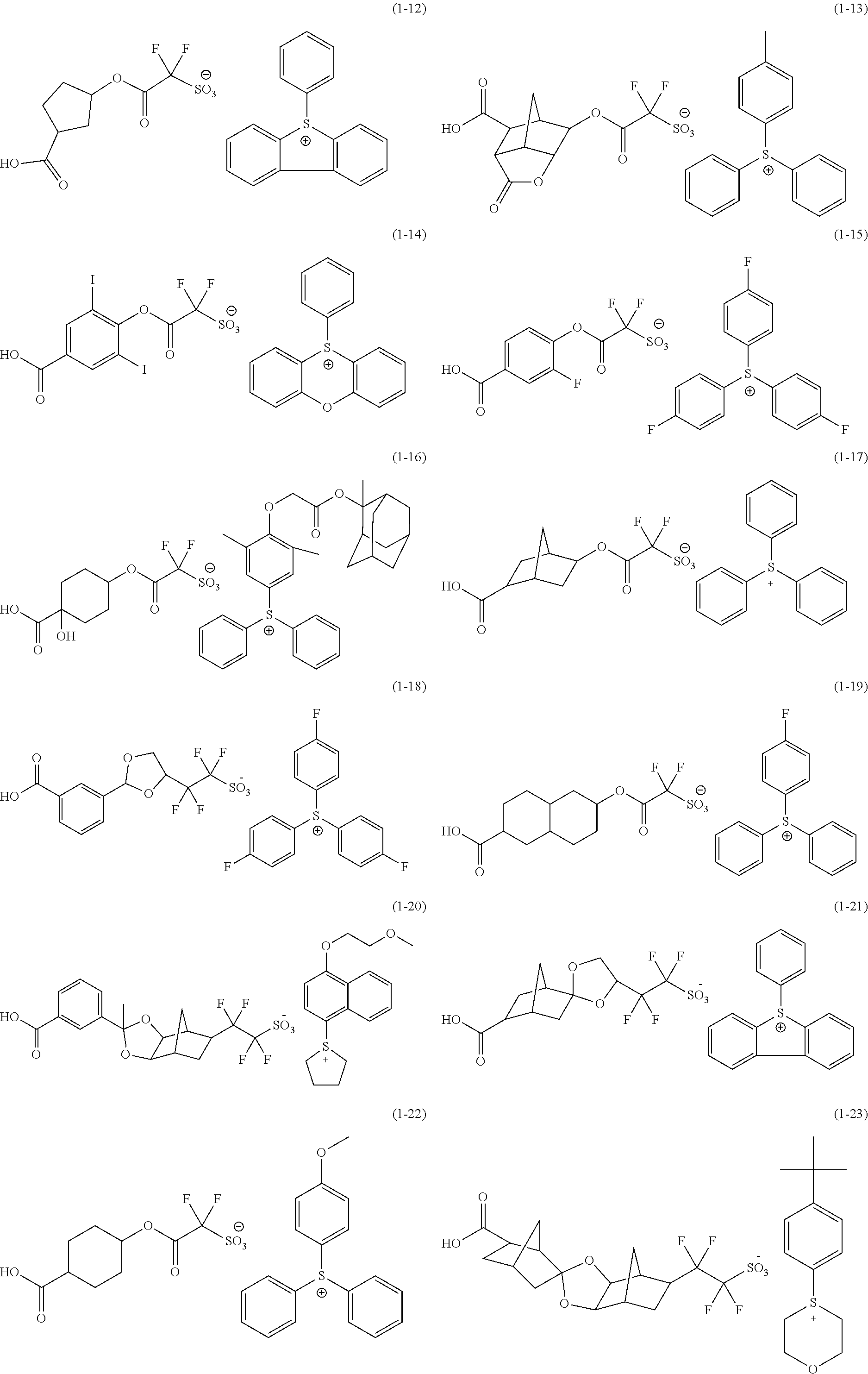

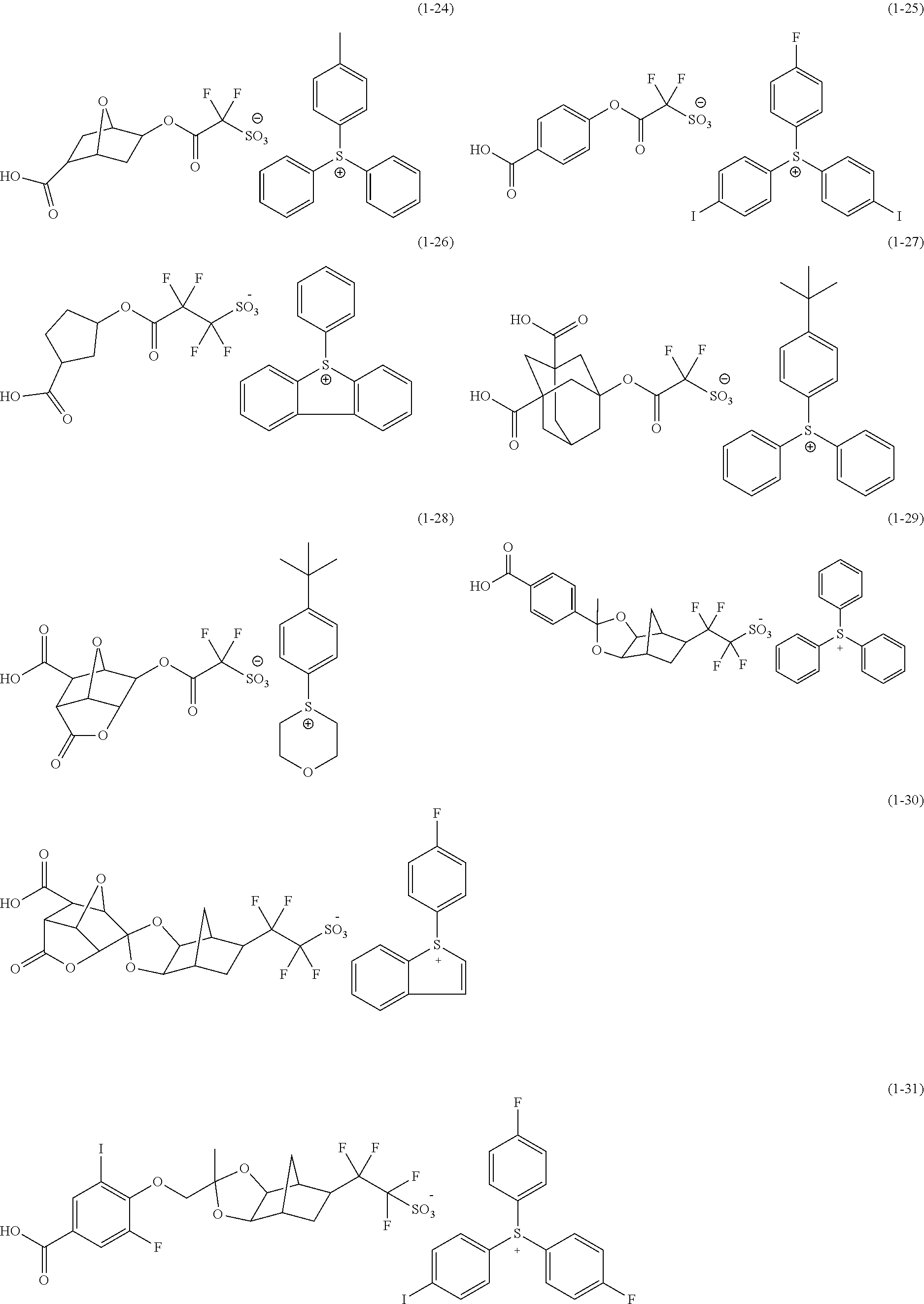

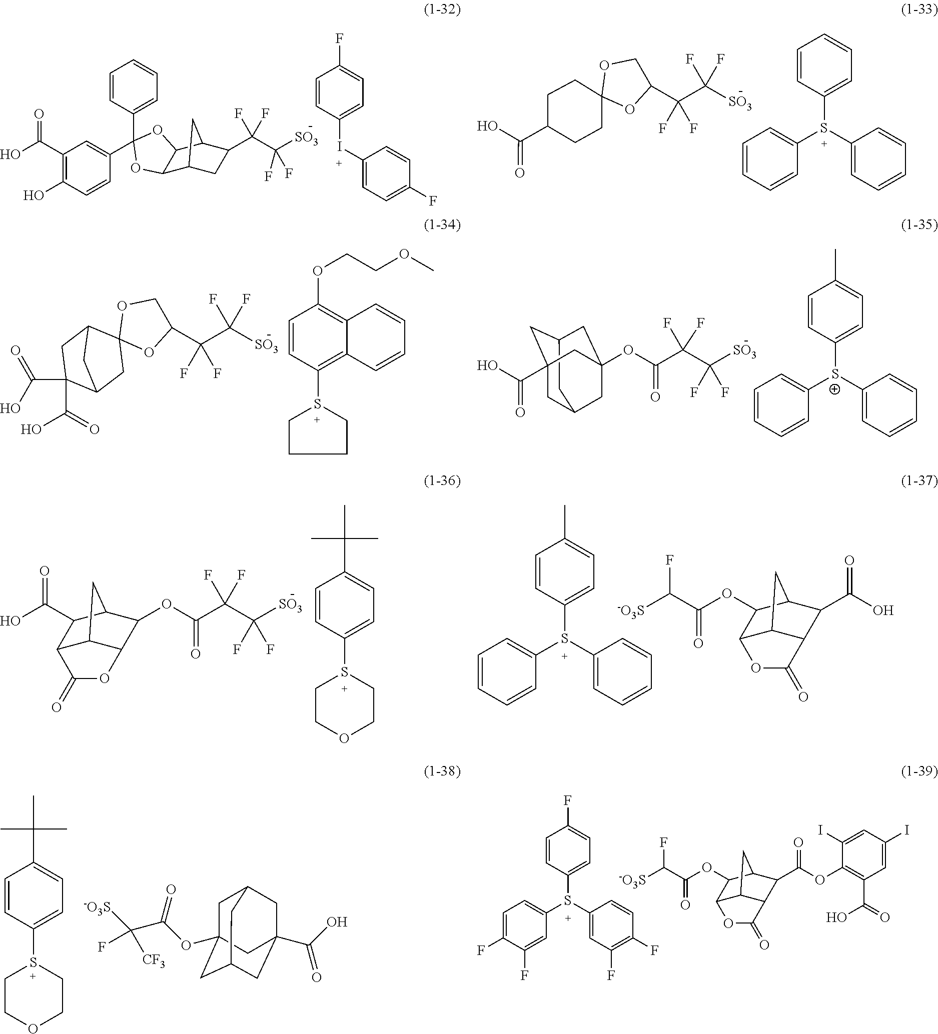

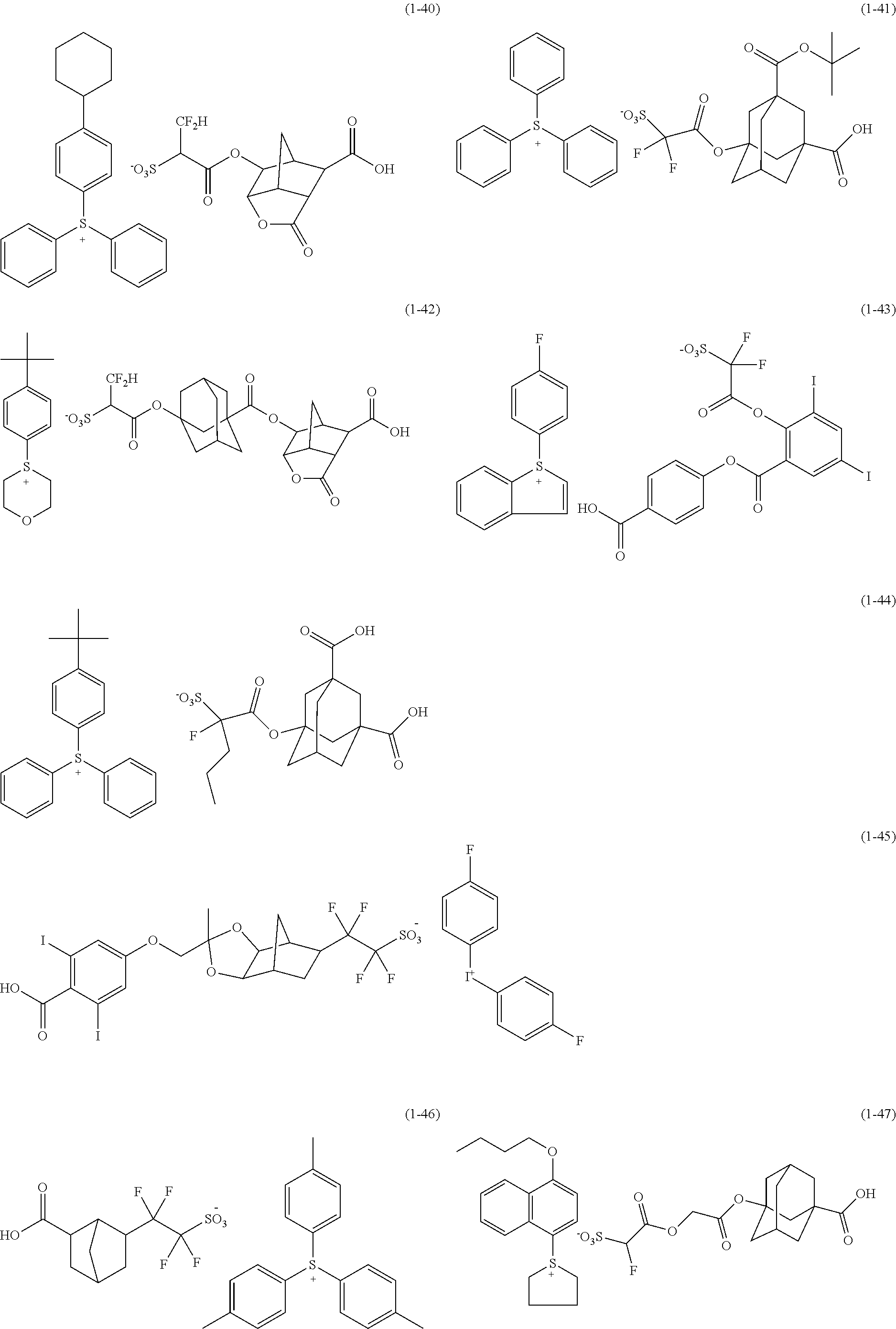

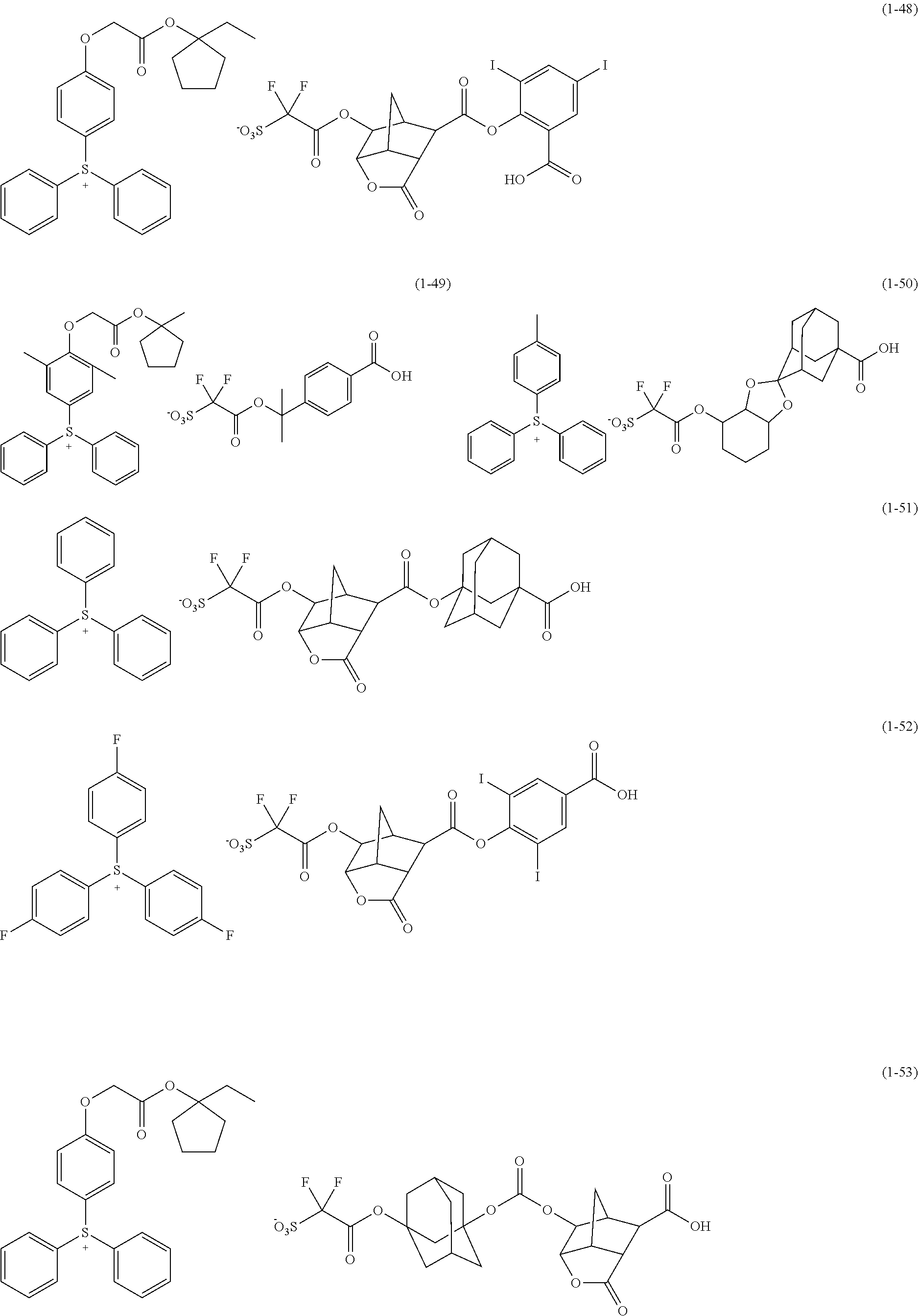

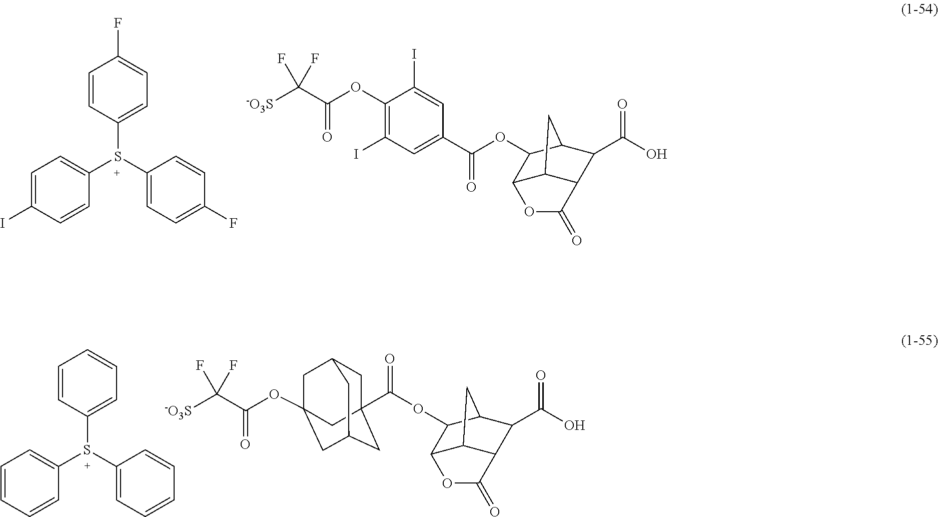

The lower limit of the content of the onium salt compound (when plural kinds of onium salt compounds are contained, the total content thereof) is preferably 1 part by mass, more preferably 2 parts by mass, and still more preferably 3 parts by mass based on 100 parts by mass of the polymer described later. The upper limit of the content is preferably 60 parts by mass, more preferably 40 parts by mass, and still more preferably 35 parts by mass. The content of the onium salt compound is appropriately selected depending on the type of a polymer to be used, exposure conditions, required sensitivity, and the like. This makes it possible to exhibit superior sensitivity, CDU performance, pattern circularity, development defect suppression performance, depth of focus, LWR performance, pattern rectangularity, and exposure latitude when forming a resist pattern.

### (Method for Synthesizing Onium Salt Compound)

As a method for synthesizing the onium salt compound, a target onium salt compound can be synthesized by performing skeleton formation of an organic acid anion, an introduction reaction of a sulfonate structure between a sulfite and an alkyl halide, and salt exchange for introduction of an onium cation in an appropriate order. Known reactions such as an esterification reaction, an acetalization reaction between a diol and a ketone, and an addition cyclization reaction between a conjugated diene and an alkene can be used for skeleton formation of the organic acid anion. Various onium salt compounds can be synthesized by appropriately selecting starting materials and precursors corresponding to the organic acid anion and the onium cation.

### (Polymer)

The polymer (base polymer) is an aggregate of polymerized chains containing the structural unit (I) represented by the formula (2). The base polymer preferably contains a structural unit (hereinafter also referred to as a “structural unit (II)”) containing an acid-dissociable group, other than the structural unit (I). The base polymer may contain another structural unit other than the structural units (I) and (II). Each of the structural units will be described below.

### [Structural Unit (I)]

The structural unit (I) is a structural unit represented by the formula (2). The base polymer may contain one structural unit (I) or two or more structural units (I).

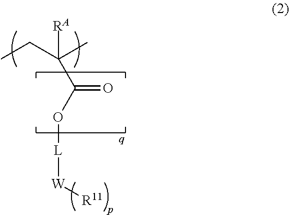

In the formula (2),
R^{A} is a hydrogen atom, a fluorine atom, a methyl group, or a trifluoromethyl group,
L is a single bond or a divalent linking group,
W is a monocyclic lactone structure, a monocyclic carbonate structure, a monocyclic sultone structure, a monocyclic sulfone structure, or a monocyclic ether structure (hereinafter they are also collectively referred to as “monocyclic polar structures”),
R¹¹ is a monovalent organic group having 1 to 10 carbon atoms, a cyano group, a nitro group, a hydroxy group, or an amino group, when there are a plurality of R¹¹s, the plurality of R¹¹s are each the same or different from each other,
p is an integer of 0 to 3, and
q is 0 or 1.

As R^{A}, a hydrogen atom and a methyl group are preferable from the viewpoint of the copolymerizability of a monomer that affords the structural unit (I), and a methyl group is more preferable.

Examples of the divalent linking group represented by L include a divalent linear or branched hydrocarbon group having 1 to 10 carbon atoms; a divalent alicyclic hydrocarbon group having 4 to 12 carbon atoms; a divalent aromatic hydrocarbon group having 6 to 12 carbon atoms; and a group composed of one or more of these hydrocarbon groups and at least one group of —CO—, —O—, —NH—, and —S—.

L is preferably a single bond or a divalent alkanediyl group having 1 to 10 carbon atoms, more preferably a single bond or a divalent alkanediyl group having 1 to 5 carbon atoms, still more preferably a single bond or a methanediyl group.

The lower limit of the carbon number in the monocyclic polar structure represented by W is preferably 3, more preferably 4. The upper limit of the carbon number is preferably 6, more preferably 5. This makes it possible to improve the solubility in an alkaline developer.

The monocyclic lactone structure represented by W is suitably represented by the formula (H-3) shown in the anion moiety of the onium salt compound. Similarly, the monocyclic carbonate structure represented by W is suitably represented by the formula (H-4), and the monocyclic sultone structure is suitably represented by the formula (H-8). Preferable examples of the monocyclic ether structure include an oxirane structure, an oxetane structure, and a tetrahydrofuran structure.

W is preferably a monocyclic lactone structure from the viewpoint of solubility in an alkaline developer in the case of development with an alkaline aqueous solution, and from the viewpoint of dissolution inhibition in an organic solvent developer in the case of development using an organic solvent. The bulkiness is thus suppressed, the polymer easily approaches another polymer or the onium salt compound, and the interaction ability between the components is improved. By the synergistic effect of the above, the diffusion length of the generated acid can be specifically shortened, and the occurrence of roughness such as variations in pattern shape and unevenness can be remarkably suppressed.

As the monovalent organic group having 1 to 10 carbon atoms represented by R¹¹, a chain hydrocarbon group having 1 to carbon atoms, a group having the above-mentioned divalent heteroatom-containing linking group between carbons of the chain hydrocarbon group, a group obtained by substituting some or all of hydrogen atoms of the chain hydrocarbon group with the above-mentioned monovalent heteroatom-containing substituent, or a combination thereof can be suitably employed together with or in place of a group corresponding to 1 to 10 carbon atoms among the monovalent cyclic organic groups having 1 to 40 carbon atoms represented by A of the above formula (1).

p is preferably an integer of 0 to 2, more preferably 0 or 1.

Specific examples of the structural unit (I) include, but are not particularly limited to, structures represented by the following formulas (2-1) to (2-34).

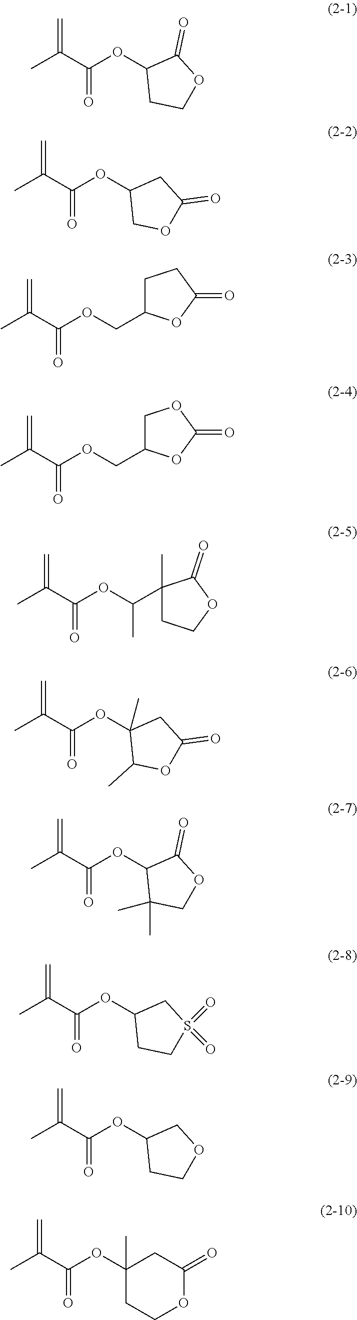

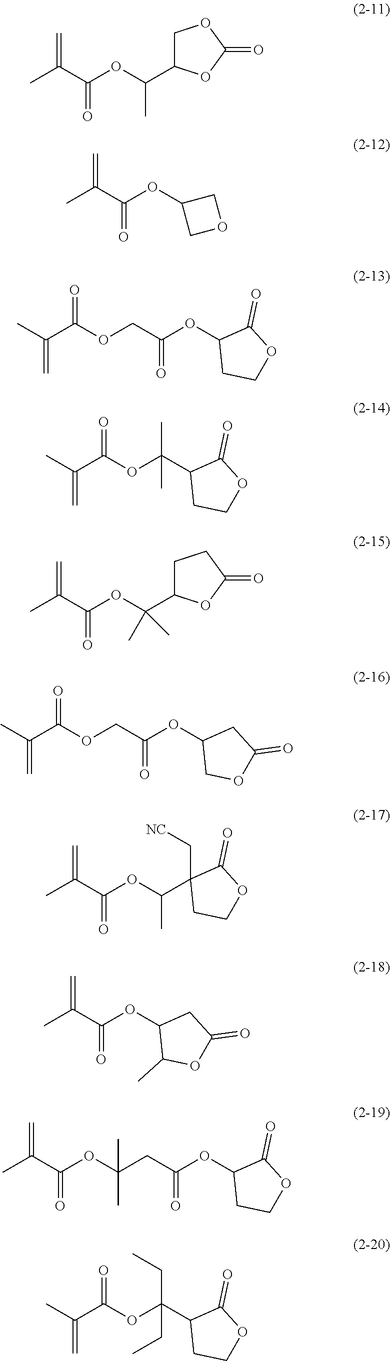

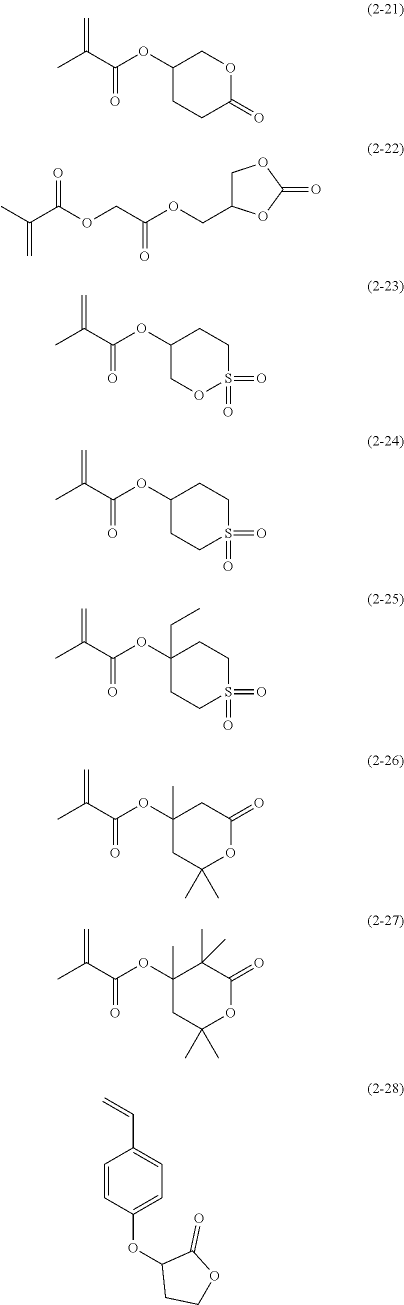

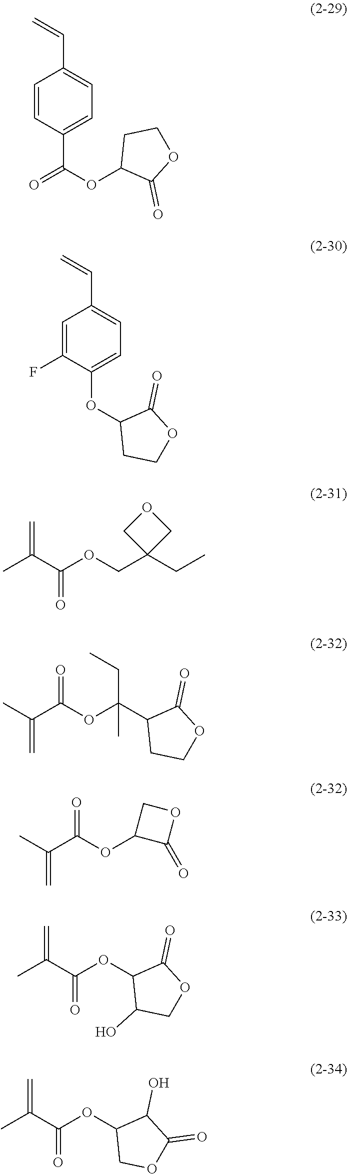

The lower limit of the content ratio of the structural unit (I) (the total content ratio when a plurality of structural units (I) are contained) is preferably 1 mol %, more preferably 3 mol %, still more preferably 5 mol % based on all structural units composing the base polymer. The upper limit of the content ratio is preferably 70 mol %, more preferably 65 mol %, still more preferably 60 mol %. When the content ratio of the structural unit (I) is adjusted within the above range, the solubility in an alkaline developer can be improved, and the pattern roughness can be further reduced.

### [Structural Unit (II)]

The structural unit (II) contains an acid-dissociable group. The “acid-dissociable group” refers to a group that substitutes for a hydrogen atom of a carboxy group, a phenolic hydroxyl group, an alcoholic hydroxyl group, a sulfo group, or the like, and is dissociated by the action of an acid. The radiation-sensitive composition is excellent in pattern-forming performance because the polymer has the structural unit (II).

The structural unit (I) is not particularly limited as long as it contains an acid-dissociable group. Examples of such a structural unit (I) include a structural unit having a tertiary alkyl ester moiety, a structural unit having a structure obtained by substituting the hydrogen atom of a phenolic hydroxyl group with a tertiary alkyl group, and a structural unit having an acetal bond. From the viewpoint of improving the pattern-forming performance of the radiation-sensitive composition, a structural unit represented by the formula (3) (hereinafter also referred to as a “structural unit (I-1)”) is preferred.

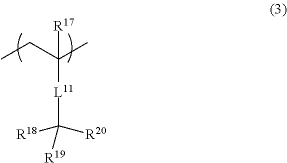

In the formula (3), R¹⁷ is a hydrogen atom, a fluorine atom, a methyl group, or a trifluoromethyl group. R¹⁸ is a monovalent hydrocarbon group having 1 to 20 carbon atoms. R¹⁹ and R²⁰ are each independently a monovalent chain hydrocarbon group having 1 to 10 carbon atoms or a monovalent alicyclic hydrocarbon group having 3 to 20 carbon atoms, or R¹⁹ and R²⁰ taken together represent a divalent alicyclic group having 3 to 20 carbon atoms together with a carbon atom to which R¹⁹ and R²⁰ are bonded. L¹¹ represents *—COO—, *-L^{11a}COO—, or *—COOL^{11a}COO—. L^{11a} is a substituted or unsubstituted alkanediyl group or arenediyl group. * is a bond to a carbon atom to which R¹⁷ is bonded.

From the viewpoint of copolymerizability of a monomer that will give the structural unit (II-1), R⁵¹ is preferably a hydrogen atom or a methyl group, more preferably a methyl group.

Examples of the alkanediyl group represented by L^{11a} include alkanediyl groups having 1 to 10 carbon atoms such as a methylene group, an ethanediyl group, a 1,3-propanediyl group, and a 2,2-propanediyl group. L^{11a} is preferably a methylene group or an ethanediyl group.

Examples of the arenediyl group represented by L^{11a} include divalent aromatic hydrocarbon groups having 6 to 20 carbon atoms such as a benzenediyl group and a naphthalenediyl group. As L^{11a}, a benzenediyl group is preferable.

Examples of the substituent that can be possessed by the arenediyl group represented by L^{11a} include a halogen atom, a hydroxy group, a carboxy group, a cyano group, a nitro group, an alkyl group, a fluorinated alkyl group, an alkoxycarbonyloxy group, an acyl group, an acyloxy group, and an alkoxy group.

Examples of the monovalent hydrocarbon group having 1 to carbon atoms represented by R¹⁸ include a chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms.

Examples of the chain hydrocarbon groups having 1 to 10 carbon atoms represented by R¹⁸ to R²⁰ include a linear or branched saturated hydrocarbon group having 1 to 10 carbon atoms or a linear or branched unsaturated hydrocarbon group having 1 to 10 carbon atoms.

As the alicyclic hydrocarbon groups having 3 to 20 carbon atoms represented by R¹⁸ to R²⁰, the monovalent alicyclic hydrocarbon groups having 3 to 20 carbon atoms in A of the formula (1) can be suitably employed.

As the monovalent aromatic hydrocarbon group having 6 to carbon atoms represented by R¹⁸, the monovalent aromatic hydrocarbon groups having 6 to 20 carbon atoms in A in the formula (1) can be suitably employed.

R¹⁸ is preferably a linear or branched saturated hydrocarbon group having 1 to 10 carbon atoms or an alicyclic hydrocarbon group having 3 to 20 carbon atoms.

The divalent alicyclic group having 3 to 20 carbon atoms formed by the chain hydrocarbon group or the alicyclic hydrocarbon group represented by R¹⁹ and R²⁰ taken together with the carbon atom to which R⁵³ and R⁵⁴ are bonded is not particularly limited as long as it is a group obtained by removing two hydrogen atoms from the same carbon atom constituting a carbon ring of a monocyclic or polycyclic alicyclic hydrocarbon having the above-described carbon number. The divalent alicyclic group having 3 to 20 carbon atoms may either be a monocyclic hydrocarbon group or a polycyclic hydrocarbon group. The polycyclic hydrocarbon group may either be a bridged alicyclic hydrocarbon group or a condensed alicyclic hydrocarbon group and may either be a saturated hydrocarbon group or an unsaturated hydrocarbon group. It is to be noted that the condensed alicyclic hydrocarbon group refers to a polycyclic alicyclic hydrocarbon group in which two or more alicyclic rings share their sides (bond between two adjacent carbon atoms).

When the monocyclic alicyclic hydrocarbon group is a saturated hydrocarbon group, preferred examples thereof include a cyclopentanediyl group, a cyclohexanediyl group, a cycloheptanediyl group, and a cyclooctanediyl group. When the monocyclic alicyclic hydrocarbon group is an unsaturated hydrocarbon group, preferred examples thereof include a cyclopentenediyl group, a cyclohexenediyl group, a cycloheptenediyl group, a cyclooctenediyl group, and a cyclodecenediyl group. The polycyclic alicyclic hydrocarbon group is preferably a bridged alicyclic saturated hydrocarbon group, and preferred examples thereof include a bicyclo[2.2.1]heptane-2,2-diyl group (norbornane-2,2-diyl group), a bicyclo[2.2.2]octane-2,2-diyl group, a tricyclo[3.3.1.1^{3,7}]decane-2,2-diyl group (adamantane-2,2-diyl group), and a tricyclo[5.2.1.0^{2,6}]decane-8,8-diyl group.

Among them, R¹⁸ is preferably an alkyl group having 1 to 4 carbon atoms, and the alicyclic structure formed by R¹⁹ and R²⁰ combined together and a carbon atom to which they are bonded is preferably a polycyclic or monocyclic cycloalkane structure.

Examples of the structural unit (II-1) include structural units represented by the formulas (3-1) to (3-15) (hereinafter also referred to as “structural units (II-1-1) to (II-1-15)”).

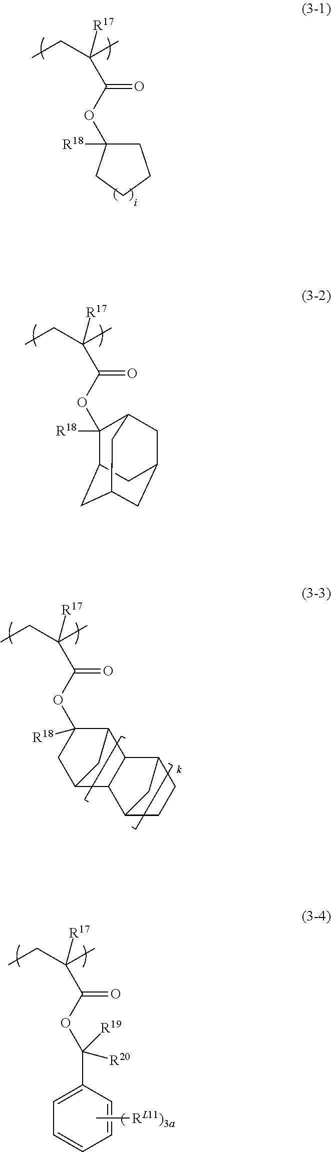

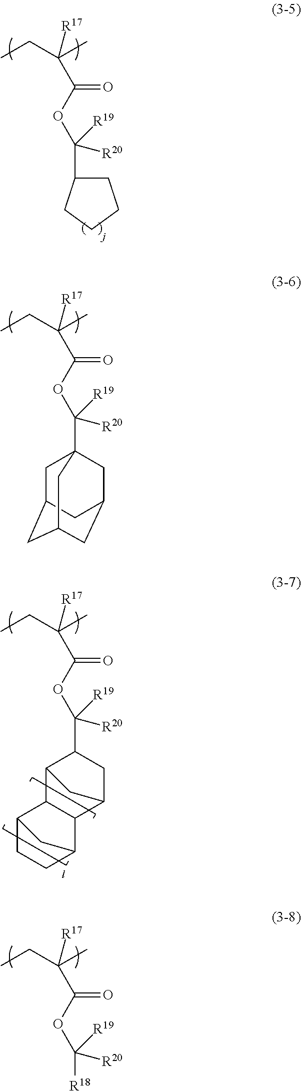

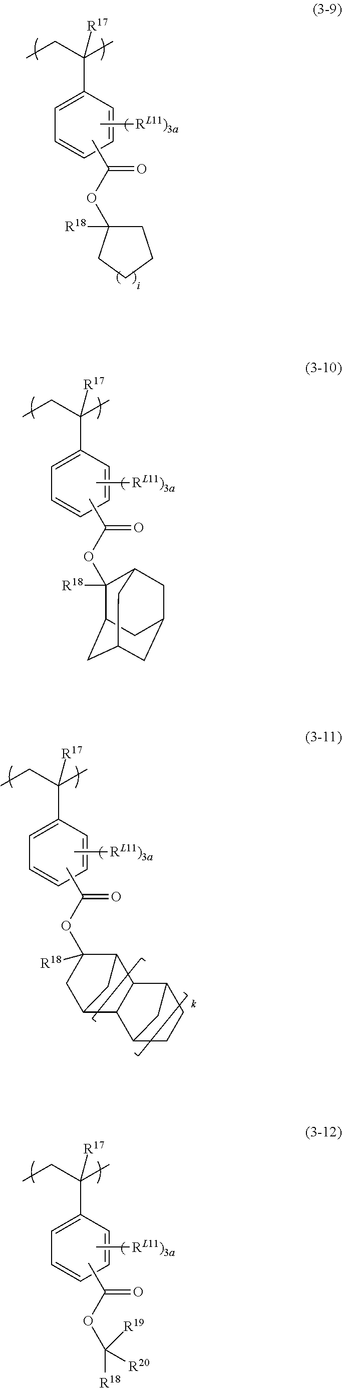

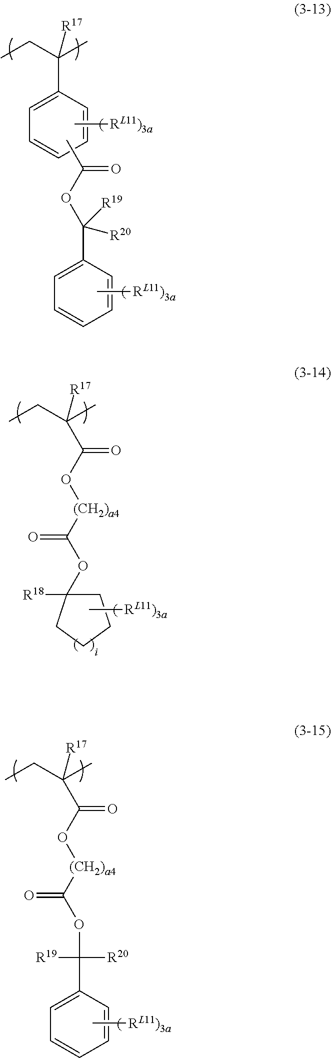

In the formulas (3-1) to (3-15), R¹⁷ to R²⁰ have the same meaning as in the formula (3). R^{L11} is a halogen atom, a hydroxy group, a carboxy group, a cyano group, a nitro group, an alkyl group, a fluorinated alkyl group, an alkoxycarbonyloxy group, an acyl group, an acyloxy group, or an alkoxy group. i and j are each independently an integer of 1 to 4. k and 1 are each 0 or 1. 3a's are each independently an integer of 0 to 3. When 3a is 2 or more, a plurality of R^{L11}s are the same as or different from each other. a4 is an integer of 1 to 3.

As i and j, 1 is preferable. R¹⁸ is preferably a methyl group, an ethyl group, an isopropyl group, a t-butyl group, an ethenyl group, a phenyl group, or an iodophenyl group. As R¹⁹ and R²⁰, a methyl group, an ethyl group, and an isopropyl group are preferable. By employing an iodine atom as R^{L11}, an iodo group can be suitably introduced into the structural unit (II).

Furthermore, the polymer may contain structural units represented by formulas (1f) to (2f) below as the structural unit (II).

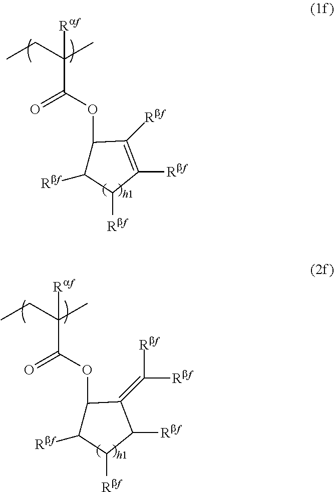

In the formulas (1f) to (2f), R^{αf}s are each independently a hydrogen atom, a fluorine atom, a methyl group, or a trifluoromethyl group. R^{βf}s are each independently a hydrogen atom or a chain alkyl group having 1 to 5 carbon atoms. h1 is an integer of 1 to 4.

As the R^{βf}, a hydrogen atom, a methyl group, or an ethyl group is preferable. h1 is preferably 1 or 2.

The base polymer may contain one type or a combination of two or more types of the structural units (II).

The lower limit of the content by percent of the structural unit (II) (a total content by percent when a plurality of types are contained) is preferably 10 mol %, more preferably 20 mol %, and still more preferably 30 mol % based on all structural units constituting the base polymer. The upper limit of the content by percent is preferably 80 mol %, more preferably 70 mol %, and still more preferably 60 mol %. When the content of the structural unit (II) is set to fall within the above range, the pattern-forming performance of the radiation-sensitive composition can further be improved.

### [Structural Unit (III)]

The structural unit (III) is a structural unit containing at least one structure selected from the group consisting of a polycyclic lactone structure, a polycyclic carbonate structure, and a polycyclic sultone structure. The structural unit (III) is an arbitrary structural unit. Since the base polymer further has the structural unit (III), the etching resistance of the resist film can be improved while the solubility into a developer is adjusted.

Examples of the structural unit (III) include structural units represented by the following formulas (T-1) to (T-6).

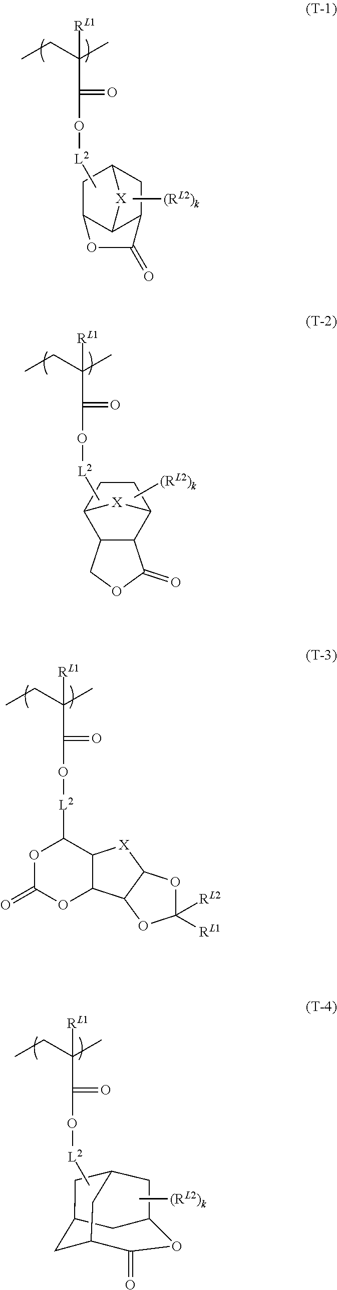

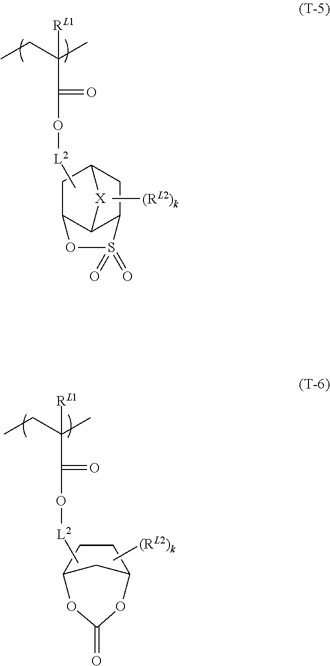

In the formulas, R^{L1} is a hydrogen atom, a fluorine atom, a methyl group, or a trifluoromethyl group. R^{L2} to R^{L3} are each independently a hydrogen atom, an alkyl group having 1 to 4 carbon atoms, a cyano group, a trifluoromethyl group, a methoxy group, a methoxycarbonyl group, a hydroxy group, a hydroxymethyl group, or a dimethylamino group. L² is a single bond or a divalent linking group. X is an oxygen atom or a methylene group. k is an integer of 0 to 3.

As the divalent linking group represented by L², a divalent linking group represented by L in the above formula (2) can be suitably employed.

Among them, a structural unit containing a lactone structure is preferable as the structural unit (III), a structural unit containing a norbornane lactone structure is more preferable, and a structural unit derived from norbornane lactone-yl (meth)acrylate is still more preferable.

When the base polymer contains the structural unit (III), the lower limit of the content ratio of the structural unit (III) is preferably 2 mol %, more preferably 5 mol %, still more preferably 8 mol % with respect to all structural units constituting the base polymer. The upper limit of the content ratio is preferably 60 mol %, more preferably 50 mol %, still more preferably 45 mol %. When the content ratio of the structural unit (III) is adjusted within the above range, the lithographic performance, such as resolution, of the radiation-sensitive composition and the adhesion between a resist pattern to be formed and a substrate can be improved.

### [Structural Unit (IV)]

The structural unit (IV) is a structural unit containing a polar group (excluding those corresponding to the structural unit (III)). The structural unit (IV) is an arbitrary structural unit. When the base polymer further has a structural unit (IV), solubility in the developer can be adjusted. As a result, lithographic performance such as resolution of the radiation-sensitive composition can be improved. Examples of the polar group include a hydroxy group, a carboxy group, a cyano group, a nitro group, and a sulfonamide group. Among them, a hydroxy group and a carboxy group are preferable, and a hydroxy group is more preferable.

Examples of the structural unit (IV) include structural units represented by the formulas.

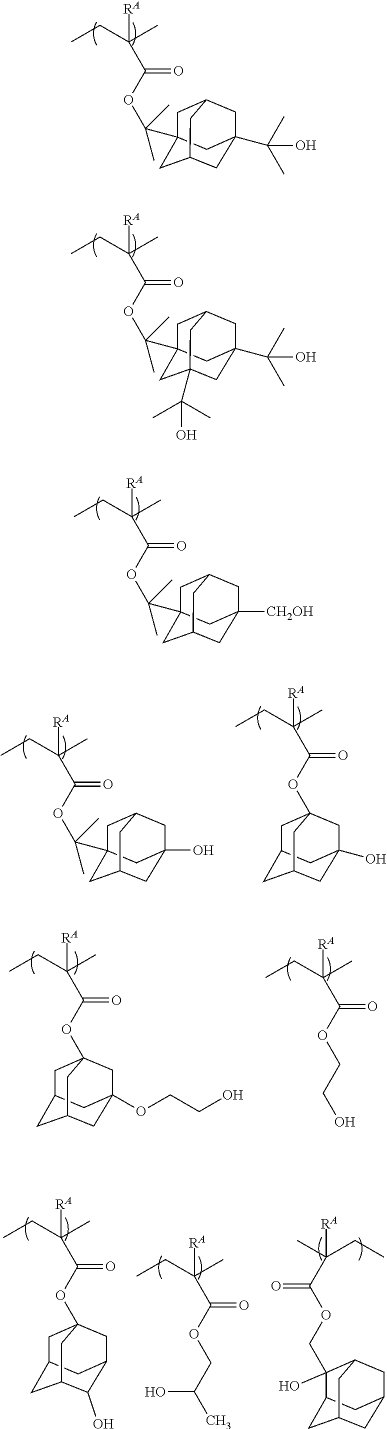

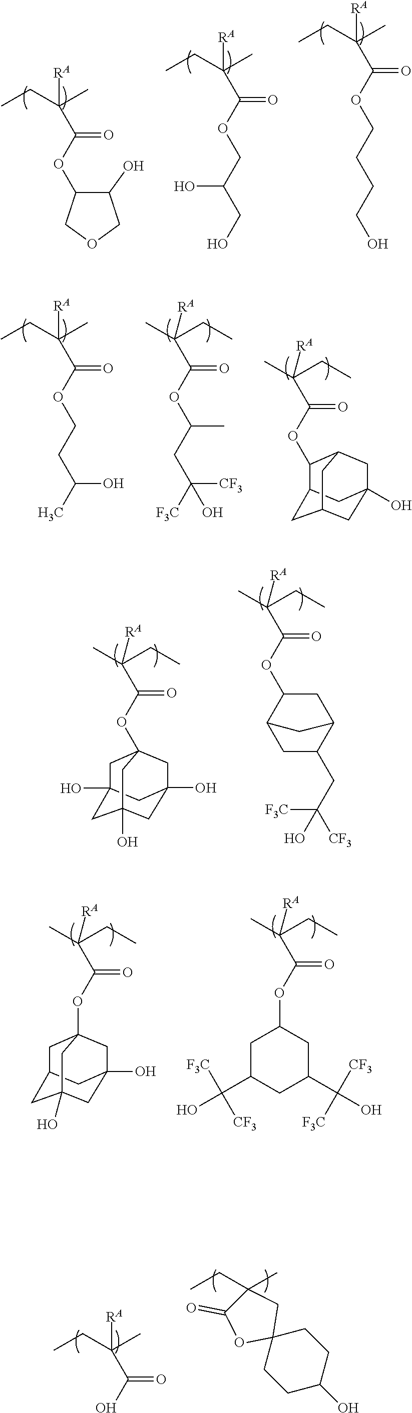

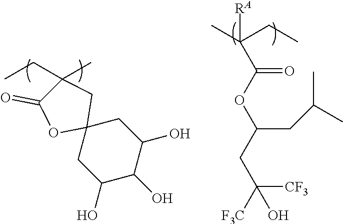

In the formulas, R^{A} is a hydrogen atom, a fluorine atom, a methyl group, or a trifluoromethyl group.

When the base polymer has the structural unit (IV) having a polar group, the lower limit of the content by percent of the structural unit (IV) is preferably 5 mol %, more preferably 8 mol %, and still more preferably 10 mol % based on all structural units constituting the base polymer. The upper limit of the content by percent is preferably 60 mol %, more preferably 50 mol %, and still more preferably 40 mol %. When the content of the structural unit (III) is set to fall within the above range, the radiation-sensitive composition can provide further improved lithography properties such as the resolution.

### [Structural Unit (V)]

The base polymer optionally has, as another structural unit, a structural unit having a phenolic hydroxyl group (hereinafter also referred to as “structural unit (V)”), in addition to the structural unit (V) having a polar group. The structural unit (V) contributes to an improvement in etching resistance and an improvement in a difference in solubility of a developer (dissolution contrast) between an exposed part and a non-exposed part. In particular, the structural unit (V) can be suitably applied to pattern formation using exposure with a radioactive ray having a wavelength of 50 nm or less, such as an electron beam or EUV. In this case, the polymer preferably has the structural units (I) and (II) together with the structural unit (V).

The structural unit containing a phenolic hydroxy group is represented by, for example, the formulas (4-1) to (4-2).

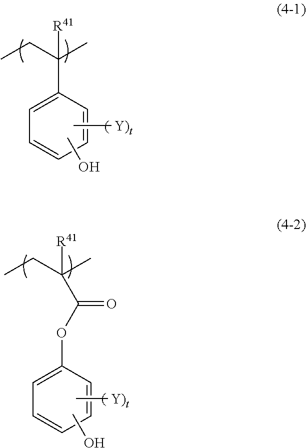

In the formulas (4-1) to (4-2), R⁴¹ is independently at each occurrence a hydrogen atom, a fluorine atom, a methyl group, or a trifluoromethyl group. Y is a halogen atom, a trifluoromethyl group, a cyano group, an alkyl or alkoxy group having 1 to 6 carbon atoms, or an acyl, acyloxy, or alkoxycarbonyl group having 2 to 7 carbon atoms. When there are a plurality of Y's, the plurality of Y's are the same or different from each other, t is an integer of 0 to 4.

When the structural unit (V) is obtained, it is preferable to obtain the structural unit (V) by polymerizing the corresponding monomer in a state where the phenolic hydroxy group is protected by a protecting group such as an alkali-dissociable group (e.g., an acyl group) during polymerization, and then deprotecting the polymerized product by hydrolysis. The polymerization of the corresponding monomer may be carried out without protecting the phenolic hydroxyl group.

In the case of a polymer for exposure to radiation having a wavelength of 50 nm or less, the lower limit of the content by percent of the structural unit (V) is preferably 10 mol %, and more preferably 20 mol % based on all structural units constituting the polymer. The upper limit of the content by percent is preferably 70 mol %, and more preferably 60 mol %.

### [Other Structural Unit]

The base polymer may contain a structural unit having an alicyclic structure represented by the formula (6) (hereinafter also referred to as “structural unit (VIII)”) as a structural unit other than the structural units listed above.

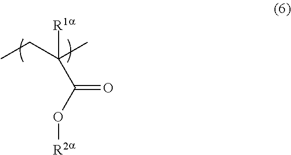

In the formula (6), R^{1α} represents a hydrogen atom, a fluorine atom, a methyl group, or a trifluoromethyl group, and R^{2α} represents a monovalent alicyclic hydrocarbon group having 3 to 20 carbon atoms.

In the formula (6), as the monovalent alicyclic hydrocarbon group having 3 to 20 carbon atoms represented by R^{2α}, the monovalent alicyclic hydrocarbon groups having 3 to 20 carbon atoms represented by A in the formula (1) can be suitably employed.

When the base polymer contains the structural unit (VIII), the lower limit of the content by percent of the structural unit (VIII) is preferably 2 mol %, more preferably 5 mol %, and still more preferably 8 mol % based on all structural units constituting the base polymer. The upper limit of the content by percent is preferably 30 mol %, more preferably 20 mol %, and still more preferably 15 mol %.

### (Synthesis Method of Base Polymer)

For example, the base polymer can be synthesized by performing a polymerization reaction of each monomer for providing each structural unit with a radical polymerization initiator or the like in a suitable solvent.

Examples of the radical polymerization initiator include an azo-based radical initiator, including azobisisobutyronitrile (AIBN), 2,2′-azobis(4-methoxy-2,4-dimethylvaleronitrile), 2,2′-azobis(2-cyclopropylpropanenitrile), 2,2′-azobis(2,4-dimethylvaleronitrile), and dimethyl 2,2′-azobisisobutyrate; and peroxide-based radical initiator, including benzoyl peroxide, t-butyl hydroperoxide, and cumene hydroperoxide. Among them, AIBN or dimethyl 2,2′-azobisisobutyrate is preferred. The radical initiator may be used alone, or two or more radical initiators may be used in combination.

Examples of the solvent used for the polymerization reaction include
alkanes including n-pentane, n-hexane, n-heptane, n-octane, n-nonane, and n-decane;
cycloalkanes including cyclohexane, cycloheptane, cyclooctane, decalin, and norbornane;
aromatic hydrocarbons including benzene, toluene, xylene, ethylbenzene, and cumene;
halogenated hydrocarbons including chlorobutanes, bromohexanes, dichloroethanes, hexamethylenedibromide, and chlorobenzenes;
saturated carboxylate esters, including ethyl acetate, n-butyl acetate, i-butyl acetate, and methyl propionate;
polyhydric alcohol partial ether acetate-based solvents, such as diethylene glycol mono-n-butyl ether acetate, propylene glycol monomethyl ether acetate, and dipropylene glycol monomethyl ether acetate;
ketones such as acetone, 2-butanone (methyl ethyl ketone), 4-methyl-2-pentanone, 2-heptanone, and cyclohexanone;
ethers such as tetrahydrofuran, dimethoxyethane, diethoxyethane, and 1,4-dioxane;
alcohols such as methanol, ethanol, 1-propanol, 2-propanol, 4-methyl-2-pentanol, and 1-methoxy-2-propanol; and
lactones such as y-butyrolactone. The solvents to be used in the polymerization may be used singly, or two or more thereof may be used in combination.

The reaction temperature of the polymerization reaction is typically from 40° C. to 150° C., and preferably from 50° C. to 120° C. The reaction time is typically from 1 hour to 48 hours, and preferably from 1 hour to 24 hours.

The molecular weight of the base polymer is not particularly limited, but the lower limit of the weight average molecular weight (Mw) as determined by Gel Permeation Chromatography (GPC) relative to standard polystyrene is preferably 3,000, more preferably 4,000, and still more preferably 5,000. The upper limit of the Mw is preferably 20,000, more preferably 15,000, and still more preferably 10,000. When the Mw of the base polymer is within the above range, the resulting resist film can obtain good heat resistance and developability.

For the base polymer, the ratio of Mw to the number average molecular weight (Mn) as determined by GPC relative to standard polystyrene (Mw/Mn) is typically 1 or more and 5 or less, preferably 1 or more and 3 or less, and more preferably 1 or more and 2 or less.

The Mw and Mn of the polymer in the specification are amounts measured by using Gel Permeation Chromatography (GPC) with the condition as described below.
GPC column: two G2000HXL, one G3000HXL, and one G4000HXL (all manufactured from Tosoh Corporation)
Column temperature: 40° C.
Eluting solvent: tetrahydrofuran
Flow rate: 1.0 mL/min
Sample concentration: 1.0% by mass
Sample injection amount: 100 μL
Detector: Differential Refractometer
Reference material: monodisperse polystyrene

The content by percent of the base polymer is preferably 60% by mass or more, more preferably 65% by mass or more, and still more preferably 70% by mass or more based on the total solid content of the radiation-sensitive composition.

### (Another Polymer)

The radiation-sensitive composition according to the present embodiment may contain, as another polymer, a polymer having higher content by mass of fluorine atoms than the above-described base polymer (hereinafter, also referred to as a “high fluorine-content polymer”). When the radiation-sensitive composition contains the high fluorine-content polymer, the high fluorine-content polymer can be localized in the surface layer of a resist film compared to the base polymer, which as a result makes it possible to enhance the water repellency of the surface of the resist film during immersion exposure or to perform surface modification of the resist film during EUV exposure or control of the distribution of the composition in the film. The radiation-sensitive composition may contain one or more high fluorine-content polymers.

The high fluorine-content polymer preferably has, for example, a structural unit represented by the formula (5) (hereinafter, also referred to as “structural unit (VI)”).

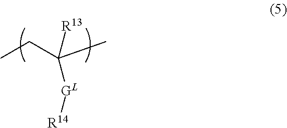

In the formula (5), R¹³ is a hydrogen atom, a methyl group, or a trifluoromethyl group; G^{L} is a single bond, an alkanediyl group having 1 to 5 carbon atoms, an oxygen atom, a sulfur atom, —COO—, —SO₂ONH—, —CONH—, —OCONH—, or a combination thereof; and R¹⁴ is a monovalent fluorinated chain hydrocarbon group having 1 to 20 carbon atoms, or a monovalent fluorinated alicyclic hydrocarbon group having 3 to 20 carbon atoms.

As R¹³ as described above, in terms of the copolymerizability of monomers resulting in the structural unit (V), a hydrogen atom or a methyl group is preferred, and a methyl group is more preferred.

As G^{L} as described above, a single bond, and —COO— are preferable, and —COO— is more preferable from the viewpoint of the copolymerizability of a monomer that gives the structural unit (V).

Example of the monovalent fluorinated chain hydrocarbon group having 1 to 20 carbon atoms represented by R¹⁴ as described above includes a group in which a part of or all of hydrogen atoms in the straight or branched chain alkyl group having 1 to 20 carbon atoms is/are substituted with a fluorine atom.

Example of the monovalent fluorinated alicyclic hydrocarbon group having 3 to 20 carbon atoms represented by R¹⁴ as described above includes a group in which a part of or all of hydrogen atoms in the monocyclic or polycyclic hydrocarbon group having 3 to 20 carbon atoms is/are substituted with a fluorine atom.

The R¹⁴ as described above is preferably a fluorinated chain hydrocarbon group, more preferably a fluorinated alkyl group, and further preferably 2,2,2-trifluoroethyl group, 2,2,3,3,3-pentafluoropropyl group, 1,1,1,3,3,3-hexafluoropropyl group and 5,5,5-trifluoro-1,1-diethylpentyl group.

When the high fluorine-content polymer has the structural unit (VI), the lower limit of the content ratio of the structural unit (VI) is preferably 50 mol %, more preferably 60 mol %, and still more preferably 70 mol % with respect to all structural units constituting the high fluorine-content polymer. The upper limit of the content ratio is preferably 95 mol %, more preferably 90 mol %, and still more preferably 85 mol %. When the content ratio of the structural unit (V) is set to fall within the above range, the content by mass of fluorine atoms of the high fluorine-content polymer can more appropriately be adjusted to further promote the localization of the high fluorine-content polymer in the surface layer of a resist film and, as a result, the water repellency of the surface of the resist film during immersion exposure can be increased, and the surface modification of the resist film during EUV exposure and the control of distribution of the composition in the film can be performed at a sufficient level.

The high fluorine-content polymer may have a fluorine atom-containing structural unit represented by the formula (f-2) (hereinafter, also referred to as a “structural unit (VII)”) in addition to or in place of the structural unit (VI). When the high fluorine-content polymer has the structural unit (VI), solubility in an alkaline developing solution is improved, and therefore generation of development defects can be prevented.

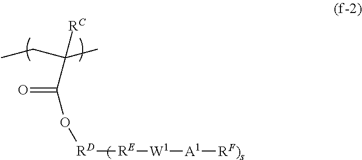

The structural unit (VII) is classified into two groups: a unit having an alkali soluble group (x); and a unit having a group (y) in which the solubility into the alkaline developing solution is increased by the dissociation by alkali (hereinafter, simply referred as an “alkali-dissociable group”). In both cases of (x) and (y), R^{c} in the formula (f-2) is a hydrogen atom, a fluorine atom, a methyl group, or a trifluoromethyl group; RD is a single bond, a hydrocarbon group having 1 to 20 carbon atoms with the valency of (s+1), a structure in which an oxygen atom, a sulfur atom, —NR^{dd}—, a carbonyl group, —COO—, —OCO—, or —CONH— is connected to the terminal on RE side of the hydrocarbon group, or a structure in which a part of hydrogen atoms in the hydrocarbon group is substituted with an organic group having a hetero atom; R^{dd} is a hydrogen atom, or a monovalent hydrocarbon group having 1 to carbon atoms; and s is an integer of 1 to 3.

When the structural unit (VII) has the alkali soluble group (x), R^{F} is a hydrogen atom; Al is an oxygen atom, —COO—* or —SO₂O—*; * refers to a bond to R^{F}; W¹ is a single bond, a hydrocarbon group having 1 to 20 carbon atoms, or a divalent fluorinated hydrocarbon group. When Al is an oxygen atom, W¹ is a fluorinated hydrocarbon group having a fluorine atom or a fluoroalkyl group on the carbon atom connecting to A¹. R^{E} is a single bond, or a divalent organic group having 1 to 20 carbon atoms. When s is 2 or 3, a plurality of R^{E}, W¹, A¹ and R^{F} may be each identical or different. The affinity of the high fluorine-content polymer into the alkaline developing solution can be improved by including the structural unit (VI) having the alkali soluble group (x), and thereby prevent from generating the development defect. As the structural unit (VI) having the alkali soluble group (x), particularly preferred is a structural unit in which A¹ is an oxygen atom and W¹ is a 1,1,1,3,3,3-hexafluoro-2,2-methanediyl group.

When the structural unit (VII) has the alkali-dissociable group (y), R^{F} is a monovalent organic group having 1 to 30 carbon atoms; Al is an oxygen atom, —NR^{aa}—, —COO—*, —OCO—*, or —SO₂O—*; R^{aa} is a hydrogen atom, or a monovalent hydrocarbon group having 1 to 10 carbon atoms; * refers to a bond to R^{F}; W¹ is a single bond, or a divalent fluorinated hydrocarbon group having 1 to 20 carbon atoms; R^{E} is a single bond, or a divalent organic group having 1 to 20 carbon atoms. When Al is —COO—*, —OCO—* or —SO₂O—*, W¹ or R^{F} has a fluorine atom on the carbon atom connecting to Al or on the carbon atom adjacent to the carbon atom. When Al is an oxygen atom, W¹ and R^{E} are a single bond; R^{D} is a structure in which a carbonyl group is connected at the terminal on R^{E} side of the hydrocarbon group having 1 to 20 carbon atoms; and R^{F} is an organic group having a fluorine atom. When s is 2 or 3, a plurality of R^{E}, W¹, A¹ and R^{E} may be each identical or different. The surface of the resist film is changed from hydrophobic to hydrophilic in the alkaline developing step by including the structural unit (VI) having the alkali-dissociable group (y). As a result, the affinity of the high fluorine-content polymer into the alkaline developing solution can be significantly improved, and thereby prevent from generating the development defect more efficiently. As the structural unit (VI) having the alkali-dissociable group (y), particularly preferred is a structural unit in which A¹ is —COO—*, and R^{F} or W¹, or both is/are a group having a fluorine atom.

In terms of the copolymerizability of monomers resulting in the structural unit (VII), R^{c} is preferably a hydrogen atom or a methyl group, and more preferably a methyl group.

When R^{E} is a divalent organic group, R^{E} is preferably a group having a lactone structure, more preferably a group having a polycyclic lactone structure, and further preferably a group having a norbornane lactone structure.

When the high fluorine-content polymer has the structural unit (VII), the lower limit of the content ratio of the structural unit (VII) is preferably 40 mol %, more preferably 50 mol %, and still more preferably 55 mol % based on all structural units constituting the high fluorine-content polymer. The upper limit of the content ratio is preferably 90 mol %, more preferably 85 mol %, and still more preferably 80 mol %. When the content ratio of the structural unit (VI) is set to fall within the above range, the water repellency and surface modification properties of the resist film can be improved, and the solubility in an alkaline developer can be improved to efficiently suppress the occurrence of development defects.

### [Other Structural Unit]

The high fluorine-content polymer may contain the structural unit (II), the structural unit (IV), and the structural unit (VIII) in the base polymer, as structural units other than the structural units listed above.

When the high fluorine-content polymer has the structural unit (II), the lower limit of the content ratio of the structural unit (II) is preferably 2 mol %, more preferably mol %, and still more preferably 8 mol % based on all structural units constituting the high fluorine-content polymer. The upper limit of the content ratio is preferably 40 mol %, more preferably 30 mol %, and still more preferably 25 mol %.

When the high fluorine-content polymer has the structural unit (IV), the lower limit of the content ratio of the structural unit (IV) is preferably 5 mol %, more preferably mol %, and still more preferably 15 mol % based on all structural units constituting the high fluorine-content polymer. The upper limit of the content ratio is preferably 50 mol %, more preferably 40 mol %, and still more preferably 35 mol %.

When the high fluorine-content polymer contains the structural unit (VIII), the lower limit of the content by percent of the structural unit (VIII) is preferably 10 mol %, more preferably 20 mol %, and still more preferably 30 mol % based on all structural units constituting the high fluorine-content polymer. The upper limit of the content by percent is preferably 60 mol %, more preferably 50 mol %, and still more preferably 45 mol %.

The lower limit of the Mw of the high fluorine-content polymer is preferably 2,000, more preferably 4,000, and still more preferably 6,000. The upper limit of the Mw is preferably 20,000, more preferably 12,000, still more preferably 8,000.

The lower limit of the Mw/Mn of the high fluorine-content polymer is usually 1, and more preferably 1.1. In addition, the upper limit of the Mw/Mn is usually 5, preferably 3, and more preferably 2.

When the radiation-sensitive composition contains the high-fluorine-content polymer, the lower limit of the content of the high fluorine-containing polymer is preferably 0.5 parts by mass, more preferably 1 part by mass, and still more preferably 2 parts by mass based on 100 parts by mass of the base polymer. The upper limit of the content of the high fluorine-containing polymer is preferably 15 parts by mass, more preferably 10 parts by mass, and still more preferably 6 parts by mass.

### (Method for Synthesizing High Fluorine-Content Polymer)

The high fluorine-content polymer can be synthesized by a method similar to the above-described method for synthesizing a base polymer.

### (Acid Diffusion Controlling Agent)

If necessary, the radiation-sensitive composition may contain an acid diffusion controlling agent. The acid diffusion controlling agent has the effect of controlling a phenomenon in which an acid generated from the onium salt compound by exposure diffuses in a resist film to prevent an undesired chemical reaction in an unexposed area. In addition, the storage stability of the resulting radiation-sensitive composition is improved. Further, the resolution of a resist pattern is further improved, and it is possible to suppress a change in the line width of a resist pattern caused by variation in post-exposure time delay between exposure and a development process. That is, a radiation-sensitive composition having excellent process stability can be obtained.

As the acid diffusion controlling agent, a radiation-sensitive weak acid generator, which generates a weak acid by exposure to light, can be suitably used. The acid generated from the radiation-sensitive weak acid generator is a weak acid that does not induce dissociation of the acid-dissociable group under the conditions of dissociating the acid-dissociable group in the polymer. In the present specification, the “dissociation” of the acid-dissociable group refers to dissociation that occurs when post-exposure baking is performed at 110° C. for 60 seconds.

Examples of the radiation-sensitive weak acid generator include an onium salt compound that is decomposed by exposure to light to lose the acid diffusion controllability thereof. Examples of the onium salt compound include a sulfonium salt compound represented by the following formula (8-1) and an iodonium salt compound represented by the following formula (8-2). In addition, a compound represented by the following formula (8-3) containing a sulfonium cation and an anion in the same molecule and a compound represented by the following formula (8-4) containing an iodonium cation and an anion in the same molecule are also included.

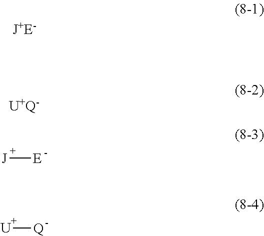

In the above formulas (8-1) to (8-4), J⁺ is a sulfonium cation, and U⁺ is an iodonium cation. Examples of the sulfonium cation represented by J⁺ include sulfonium cations represented by the formulas (X-1) to (X-4). Examples of the iodonium cation represented by U⁺ include iodonium cations represented by the formulas (X-5) and (X-6). E⁻ and Q⁻ are each independently an anion represented by OH⁻, R^{α}—COO⁻, or R^{α}—SO₃⁻. R^{α} is a single bond, or a monovalent organic group having 1 to 30 carbon atoms. As the monovalent organic group having 1 to 30 carbon atoms, the monovalent organic groups of R¹¹ in the formula (2) having, instead of 1 to 10 carbon atoms, an expanded range of carbon atoms of 1 to 30 can suitably be employed.

Examples of the radiation-sensitive weak acid generator include compounds represented by the following formulas.

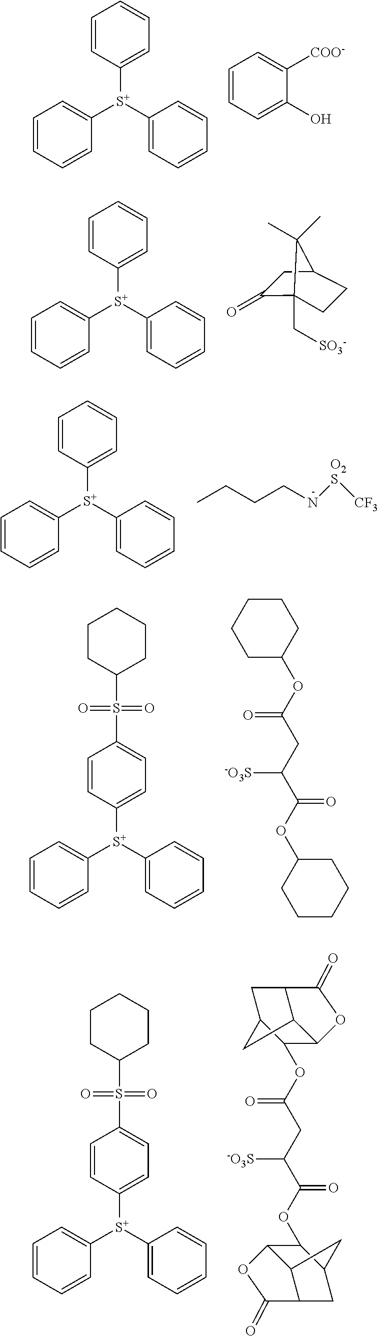

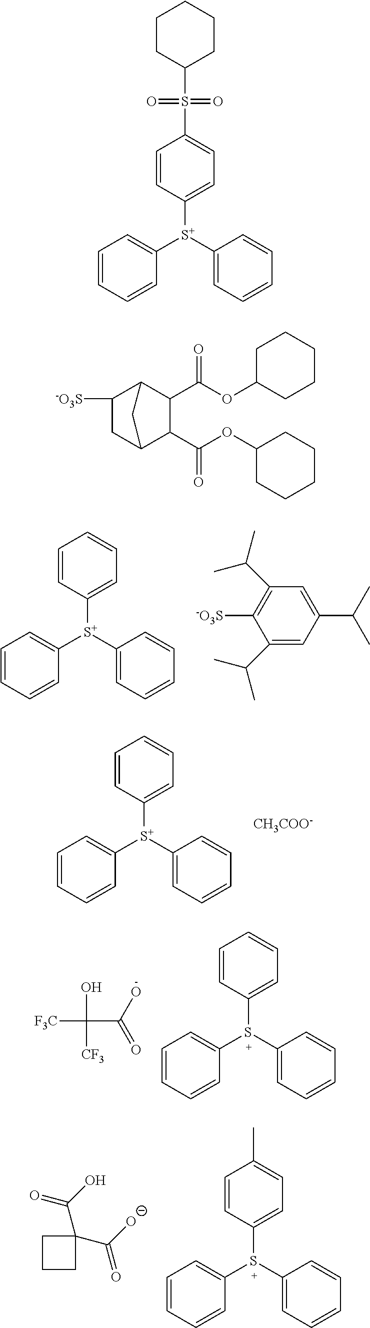

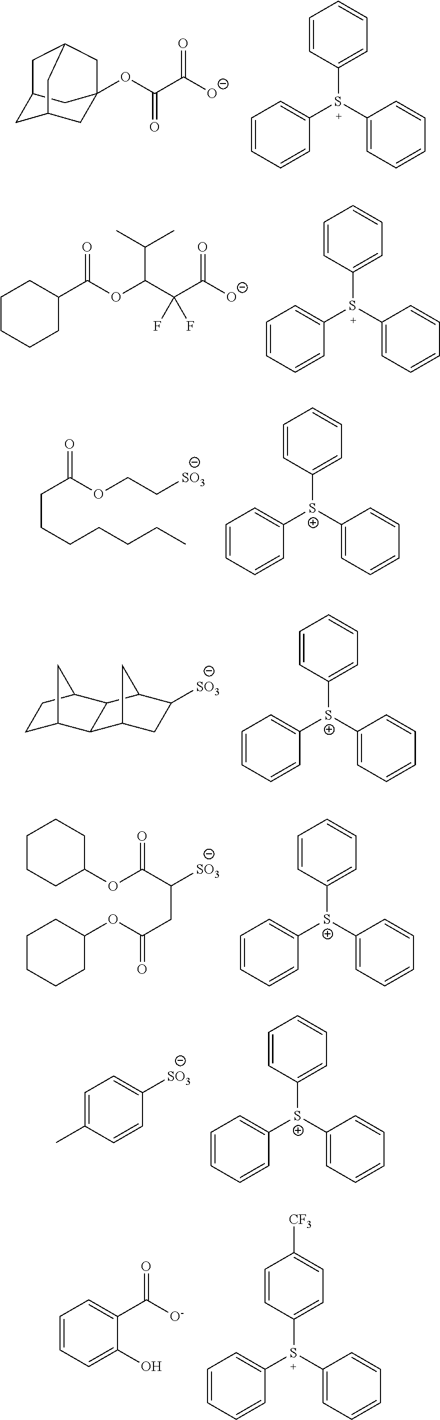

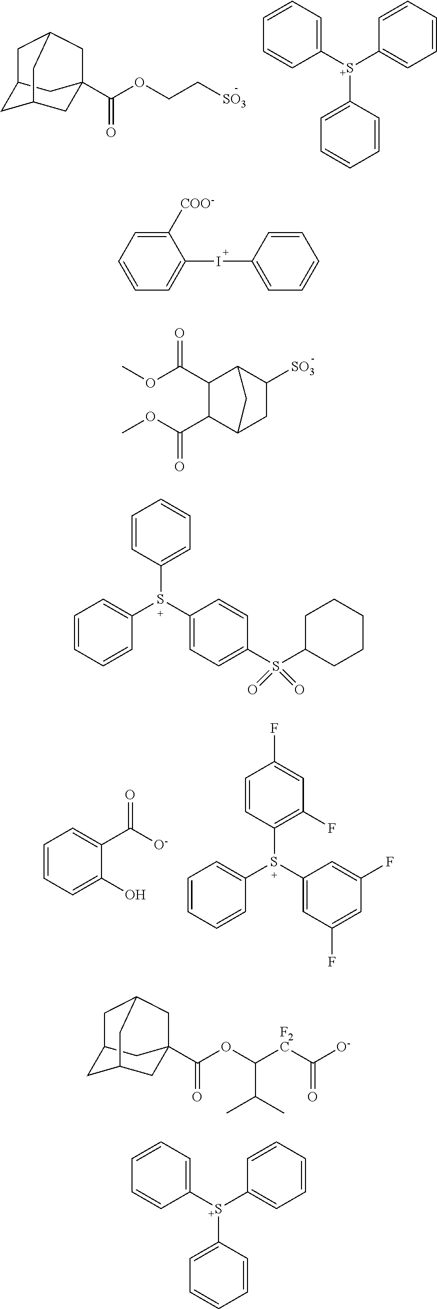

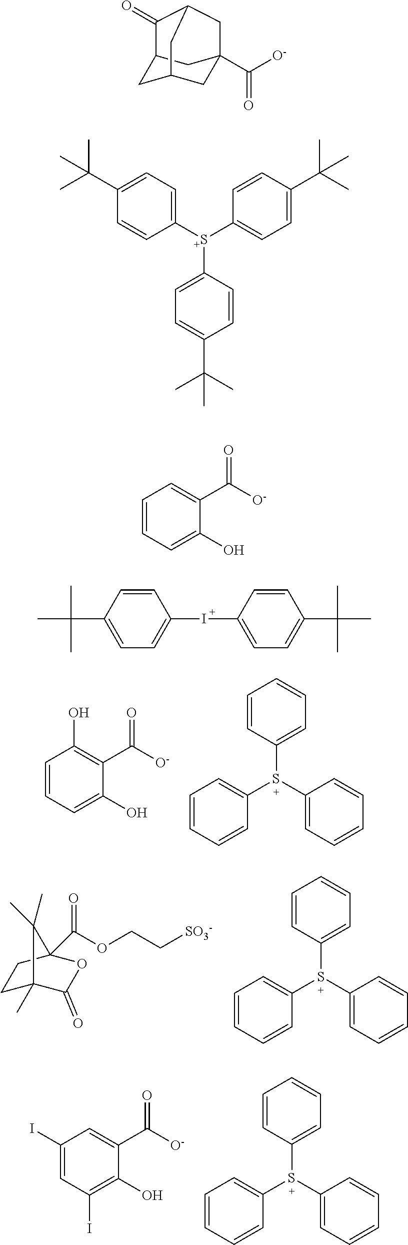

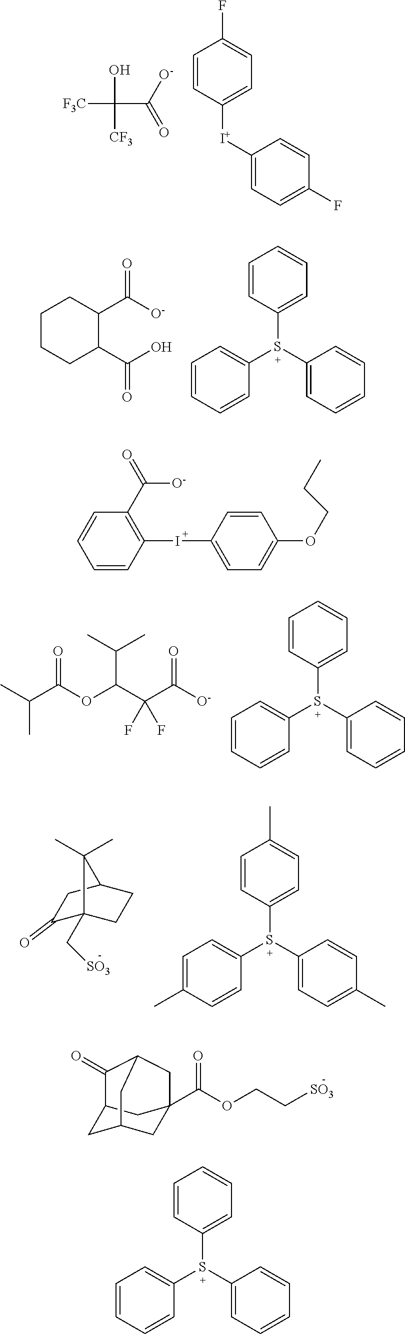

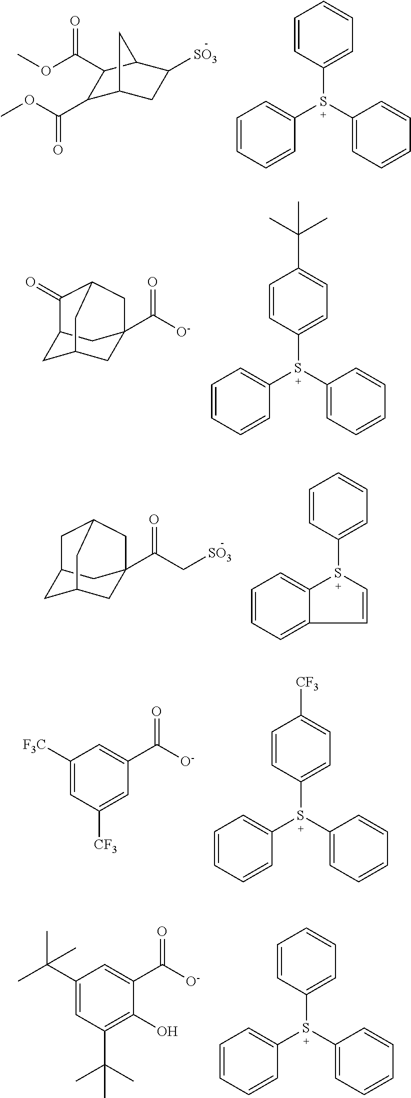

Among them, the radiation-sensitive weak acid generator is preferably a sulfonium salt, more preferably a triarylsulfonium salt, still more preferably an onium salt of triarylsulfonium and a salicylic acid anion that may have a substituent, an onium salt of triarylsulfonium and a benzoic acid anion that may have a substituent, an onium salt of triarylsulfonium and a difluorocarboxylic acid anion that may have a substituent, an onium salt of triarylsulfonium and a dicarboxylic acid monoanion that may have a substituent, an onium salt of triarylsulfonium and an adamantanecarboxylic acid anion that may have a substituent, and a triarylsulfonium 10-camphorsulfonate.

The lower limit of the content of the acid diffusion controlling agent is preferably 0.5 parts by mass, more preferably 1 part by mass, still more preferably 2 parts by mass based on 100 parts by mass of the polymer. The upper limit of the content is preferably 30 parts by mass, more preferably 20 parts by mass, still more preferably 15 parts by mass. When the content of the acid diffusion controlling agent is set within the above range, the lithographic performance of the radiation-sensitive composition can be further improved. The radiation-sensitive composition may include one type of the acid diffusion controlling agent, or two or more acid diffusion controlling agents in combination.

### (Solvent)

The radiation-sensitive composition according to the present embodiment contains a solvent. The solvent is not particularly limited as long as the solvent is a solvent capable of dissolving or dispersing at least the onium salt compound, the base polymer, and optional components or the like contained as desired.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
a monoalcohol-based solvent having 1 to 18 carbon atoms, including iso-propanol, 4-methyl-2-pentanol, n-hexanol, 2-ethylhexanol, furfuryl alcohol, cyclohexanol, 3,3,5-trimethylcyclohexanol, and diacetone alcohol;
a polyhydric alcohol having 2 to 18 carbon atoms, including ethylene glycol, 1,2-propylene glycol, 2-methyl-2,4-pentanediol, 2,5-hexanediol, diethylene glycol, dipropylene glycol, triethylene glycol, and tripropylene glycol; and
a polyhydric alcohol partial ether-based solvents obtained by etherifying a part of hydroxy groups of the polyhydric alcohol-based solvents such as 3-methoxybutanol and 1-methoxy-2-propanol (propylene glycol monomethyl ether).

In the present embodiment, the alcohol-based solvents also include alcohol acid ester-based solvents such as methyl lactate, ethyl lactate, propyl lactate, butyl lactate, methyl 2-hydroxyisobutyrate, i-propyl 2-hydroxyisobutyrate, i-butyl 2-hydroxyisobutyrate, and n-butyl 2-hydroxyisobutyrate.

Examples of the ether-based solvent include:
a dialkyl ether-based solvent, including diethyl ether, dipropyl ether, and dibutyl ether;
a cyclic ether-based solvent, including tetrahydrofuran and tetrahydropyran;
an ether-based solvent having an aromatic ring, including diphenylether and anisole (methyl phenyl ether); and
an etherized polyhydric alcohol-based solvent in which a hydroxy group in the polyhydric alcohol-based solvent is etherized.

Examples of the ketone-based solvent include:
a chain ketone-based solvent, including acetone, butanone, and methyl-iso-butyl ketone;
a cyclic ketone-based solvent, including cyclopentanone, cyclohexanone, and methylcyclohexanone; and 2,4-pentanedione, acetonylacetone, and acetophenone.

Examples of the amide-based solvent include:
a cyclic amide-based solvent, including N,N′-dimethyl imidazolidinone and N-methylpyrrolidone; and
a chain amide-based solvent, including N-methylformamide, N,N-dimethylformamide, N,N-diethylformamide, acetamide, N-methylacetamide, N,N-dimethylacetamide, and N-methylpropionamide.

Examples of the ester-based solvent include:
a monocarboxylate ester-based solvent, including n-butyl acetate;
a partially etherized polyhydric alcohol acetate-based solvent, including diethylene glycol mono-n-butyl ether acetate, propylene glycol monomethyl ether acetate, and dipropylene glycol monomethyl ether acetate;
a lactone-based solvent, including y-butyrolactone and valerolactone;
a carbonate-based solvent, including diethyl carbonate, ethylene carbonate, and propylene carbonate; and
a polyhydric carboxylic acid diester-based solvent, including propylene glycol diacetate, methoxy triglycol acetate, diethyl oxalate, ethyl acetoacetate, and diethyl phthalate.

Examples of the hydrocarbon-based solvent include:
an aliphatic hydrocarbon-based solvent, including n-hexane, cyclohexane, and methylcyclohexane;
an aromatic hydrocarbon-based solvent, including benzene, toluene, di-iso-propylbenzene, and n-amylnaphthalene.

Among these, an alcohol-based solvent, an ester-based solvent, and an ether-based solvent are preferable, an alcohol acid ester-based solvent, a polyhydric alcohol partial ether acetate-based solvent, a lactone-based solvent, and a polyhydric alcohol partial ether-based solvent are more preferable, and propylene glycol monomethyl ether acetate, γ-butyrolactone, ethyl lactate, and propylene glycol monomethyl ether are still more preferable. The radiation-sensitive composition may contain one solvent, or two or more solvents.

### (Other Optional Components)

The radiation-sensitive composition may contain other optional components other than the above-descried components. Examples of other optional components include a known radiation-sensitive acid generator other than the onium salt compound, a cross-linking agent, a localization enhancing agent, a surfactant, an alicyclic backbone-containing compound, and a sensitizer. These other optional components may be used singly or in combination of two or more of them.

### <Method for Preparing Radiation-Sensitive Composition>

The radiation-sensitive composition can be prepared by, for example, mixing the onium salt compound, the polymer, and, as necessary, optional components such as the high fluorine-content polymer and the like, as well as the solvent in a prescribed ratio. The radiation-sensitive composition is preferably filtered through, for example, a filter having a pore diameter of about 0.05 μm to 0.40 μm after mixing. The solid matter concentration of the radiation-sensitive composition is usually 0.1 mass % to 50 mass %, preferably 0.5 mass % to 30 mass %, more preferably 1 mass % to 20 mass %.

### <Method for Forming Pattern>

A pattern forming method according to an embodiment of the present disclosure includes:
a step (1) of applying the radiation-sensitive composition directly or indirectly to a substrate to form a resist film (hereinafter, also referred to as a “resist film forming step”);
a step (2) of exposing the resist film (hereinafter, also referred to as an “exposure step”); and
a step (3) of developing the exposed resist film (hereinafter, also referred to as a “developing step”).

In accordance with this method for forming a resist pattern, a high-quality resist pattern can be formed because of the use of the radiation-sensitive composition described above capable of forming a resist film superior in sensitivity, LWR performance, pattern rectangularity, CDU performance, and pattern circularity, and Exposure latitude. Hereinbelow, each of the steps will be described.

### [Resist Film Forming Step]

In this step (the above mentioned step (1)), a resist film is formed with the radiation-sensitive composition. Examples of the substrate on which the resist film is formed include one traditionally known in the art, including a silicon wafer, silicon dioxide, and a wafer coated with aluminum. An organic or inorganic antireflection film may be formed on the substrate, as disclosed in JP-B-06-12452 and JP-A-59-93448. Examples of the applicating method include a rotary coating (spin coating), flow casting, and roll coating. After applicating, a prebake (PB) may be carried out in order to evaporate the solvent in the film, if needed. The temperature of PB is typically from 60° C. to 150° C., and preferably from 80° C. to 140° C. The duration of PB is typically from 5 seconds to 600 seconds, and preferably from 10 seconds to 300 seconds.

The lower limit of the thickness of the resist film to be formed is preferably 10 nm, more preferably 15 nm, and still more preferably 20 nm. The upper limit of the film thickness is preferably 500 nm, more preferably 400 nm, and still more preferably 300 nm. In particular, when a thick resist film is exposed to ArF excimer laser light in an exposure step described later, the lower limit of the film thickness may be 100 nm, may be 150 nm, or may be 200 nm.

When the immersion exposure is carried out, irrespective of presence of a water repellent polymer additive such as the high fluorine-content polymer in the radiation-sensitive composition, the formed resist film may have a protective film for the immersion which is not soluble into the immersion liquid on the film in order to prevent a direct contact between the immersion liquid and the resist film. As the protective film for the immersion, a solvent-removable protective film that is removed with a solvent before the developing step (for example, see JP-A-2006-227632); or a developer-removable protective film that is removed during the development of the developing step (for example, see WO2005-069076 and WO2006-035790) may be used. In terms of the throughput, the developer-removable protective film is preferably used.

When the next step, the exposure step, is performed with radiation having a wavelength of 50 nm or less, it is preferable to use a polymer having the structural unit (II) and the structural unit (V) as the base polymer in the composition.

### [Exposing Step]

In this step (the above mentioned step (2)), the resist film formed in the resist film forming step as the step (1) is exposed by irradiating with a radioactive ray through a photomask (optionally through an immersion medium such as water). Examples of the radioactive ray used for the exposure include visible ray, ultraviolet ray, far ultraviolet ray, extreme ultraviolet ray (EUV); an electromagnetic wave including X ray and y ray; an electron beam; and a charged particle radiation such as a ray. Among them, far ultraviolet ray, an electron beam, or EUV is preferred. ArF excimer laser light (wavelength is 193 nm), KrF excimer laser light (wavelength is 248 nm), an electron beam, or EUV is more preferred.

When the exposure is carried out by immersion exposure, examples of the immersion liquid include water and fluorine-based inert liquid. The immersion liquid is preferably a liquid which is transparent with respect to the exposing wavelength, and has a minimum temperature factor of the refractive index so that the distortion of the light image reflected on the film becomes minimum. However, when the exposing light source is ArF excimer laser light (wavelength is 193 nm), water is preferably used because of the ease of availability and ease of handling in addition to the above considerations. When water is used, a small proportion of an additive that decreases the surface tension of water and increases the surface activity may be added. Preferably, the additive cannot dissolve the resist film on the wafer and can neglect an influence on an optical coating at an under surface of a lens. The water used is preferably distilled water.

After the exposure, post exposure bake (PEB) is preferably carried out to promote the dissociation of the acid-dissociable group in the polymer by the acid generated from the radiation-sensitive acid generator with the exposure in the exposed part of the resist film. The difference of solubility into the developer between the exposed part and the non-exposed part is generated by the PEB. The temperature of PEB is typically from 50° C. to 180° C., and preferably from 80° C. to 140° C. The duration of PEB is typically from 5 seconds to 600 seconds, and preferably from 10 seconds to 300 seconds.

### [Developing Step]

In this step (the above mentioned step (3)), the resist film exposed in the exposing step as the step (2) is developed. By this step, the predetermined resist pattern can be formed. After the development, the resist pattern is washed with a rinse solution such as water or alcohol, and the dried, in general.

Examples of the developer used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferred, and 2.38% by mass of aqueous TMAH solution is more preferred.

In the case of the development with organic solvent, examples of the solvent include an organic solvent, including a hydrocarbon-based solvent, an ether-based solvent, an ester-based solvent, a ketone-based solvent, and an alcohol-based solvent; and a solvent containing an organic solvent. Examples of the organic solvent include one, two or more solvents listed as the solvent for the radiation-sensitive composition. Among them, an ether-based solvent, an ester-based solvent or a ketone-based solvent is preferred. As the ether-based solvent, a glycol ether-based solvent is preferable, and ethylene glycol monomethyl ether and propylene glycol monomethyl ether are more preferable. The ester-based solvent is preferably an acetate ester-based solvent, and more preferably n-butyl acetate or amyl acetate. The ketone-based solvent is preferably a chain ketone, and more preferably 2-heptanone. The content of the organic solvent in the developer is preferably 80% by mass or more, more preferably 90% by mass or more, further preferably 95% by mass or more, and particularly preferably 99% by mass or more. Examples of the ingredient other than the organic solvent in the developer include water and silicone oil.

As described above, the developer may be either an alkaline developer or an organic solvent developer. The developer can be appropriately selected depending on whether the desired positive pattern or negative pattern is desired.

Examples of the developing method include a method of dipping the substrate in a tank filled with the developer for a given time (dip method); a method of developing by putting and leaving the developer on the surface of the substrate with the surface tension for a given time (paddle method); a method of spraying the developer on the surface of the substrate (spray method); and a method of injecting the developer while scanning an injection nozzle for the developer at a constant rate on the substrate rolling at a constant rate (dynamic dispense method).

### <Onium Salt Compound>

The onium salt compound is a compound represented by the following formula (1-d).

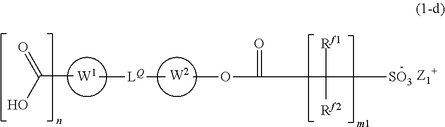

wherein,
W¹ and W² are each independently a substituted or unsubstituted cyclic structure,
L^{Q} is a single bond, *—OCO—, or *—O—CO—O—, * is a bond on the W¹ side,
R^{f1} and R^{f2} are each independently a hydrogen atom, a monovalent organic group having 1 to 20 carbon atoms, a fluorine atom, or a monovalent fluorinated hydrocarbon group, when there are a plurality of Rf's and R^{f2}s, the plurality of R^{f1}s and R^{f2}s are the same or different from each other, At least one of R^{f1} and R^{f2} is a fluorine atom or a monovalent fluorinated hydrocarbon group,
m1 is an integer of 1 to 4,
n is an integer of 1 to 3, and Z₁⁺ represents a monovalent radiation-sensitive onium cation.

As the onium salt compound, the onium salt compound represented by the formula (1-d) in the radiation-sensitive composition can be suitably employed.

Specific examples of the anion structure of the onium salt compound represented by the formula (1-d) include compounds represented by the following formulas.

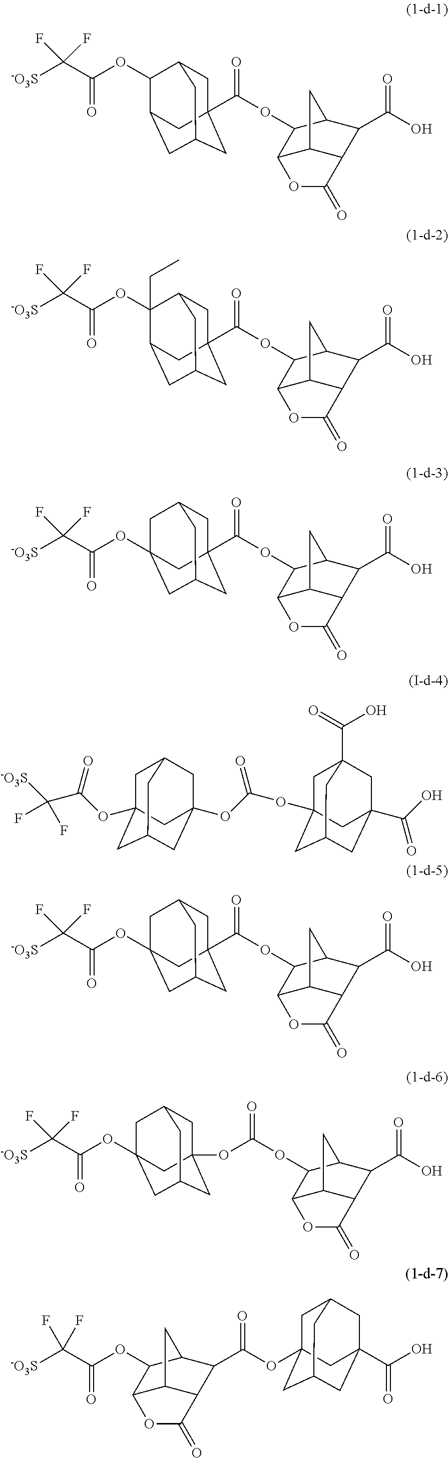

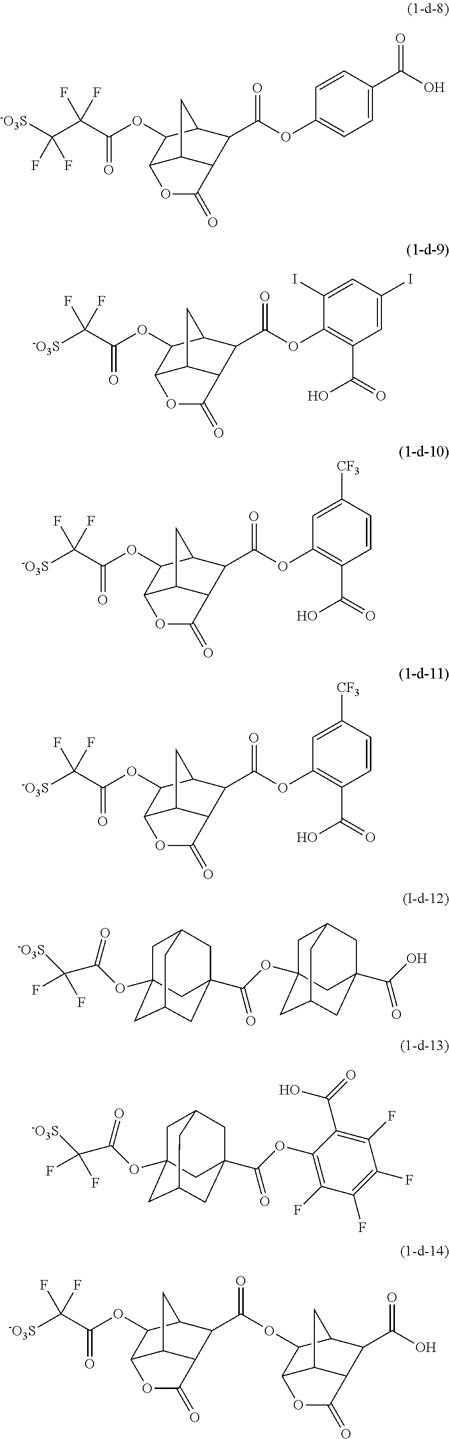

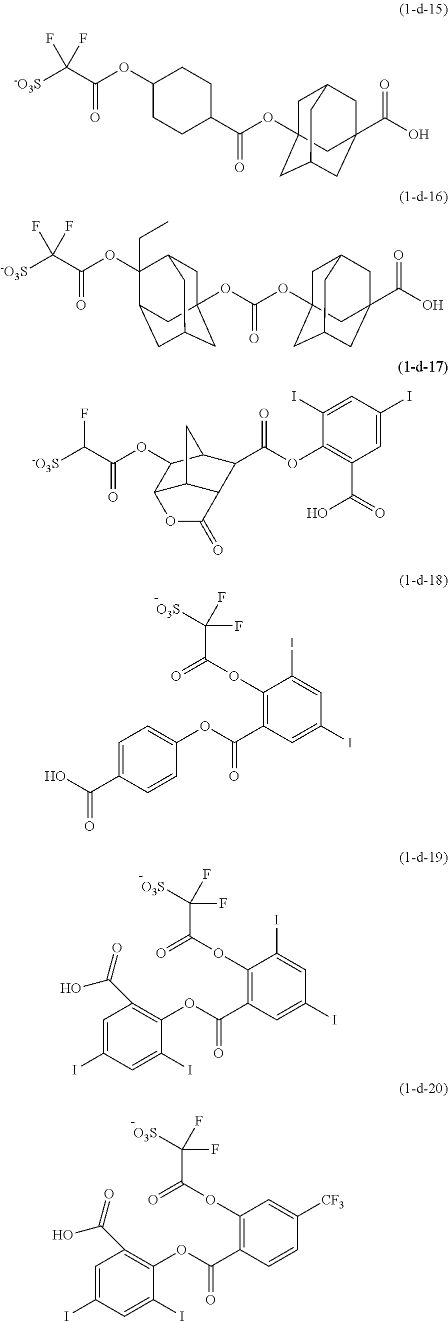

As specific examples of the monovalent radiation-sensitive onium cation represented by Z₁⁺, the structures exemplified as the specific examples of the monovalent radiation-sensitive onium cation represented by Z⁺ in the formula (1) can be suitably employed.

### EXAMPLES

Hereinafter, the present invention will be specifically described with reference to Examples, but the present invention is not limited to these Examples. Methods for measuring various physical property values are shown below.

### [Weight-Average Molecular Weight (Mw) and Number-Average Molecular Weight (Mn)]

The Mw and Mn of a polymer were measured under the conditions described above. A degree of dispersion (Mw/Mn) was calculated from results of the measured Mw and Mn.

### [¹³C-NMR Analysis]

¹³C-NMR analysis of the polymer was performed using a nuclear magnetic resonance apparatus (“JNM-Delta 400” manufactured by JEOL Ltd.).

### <Synthesis of Polymer>

Monomers used for synthesis of polymers in Examples and Comparative Examples are shown below. In the following synthesis examples, unless otherwise specified, “parts by mass” means a value taken when the total mass of the monomers used is 100 parts by mass, and “mol %” means a value taken when the total number of moles of the monomers used is 100 mol %.

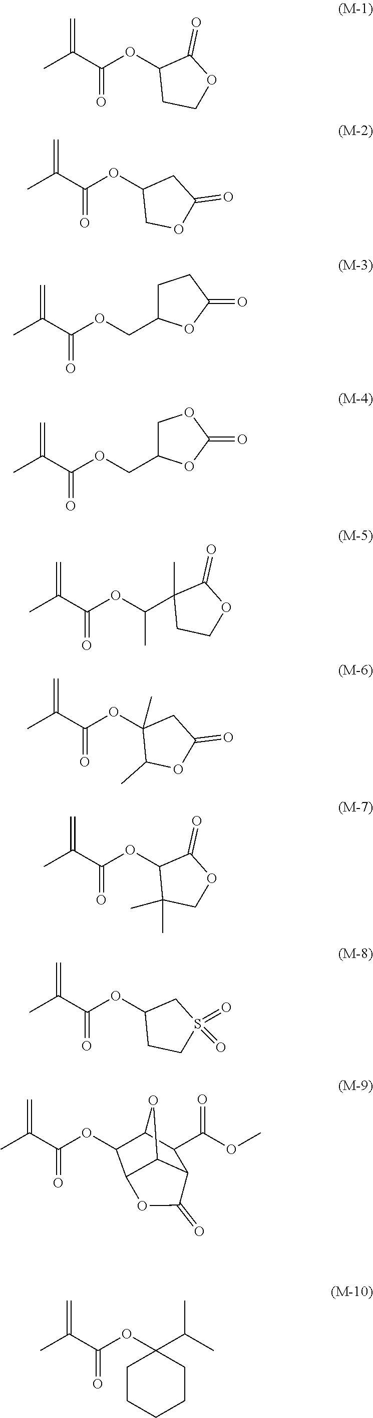

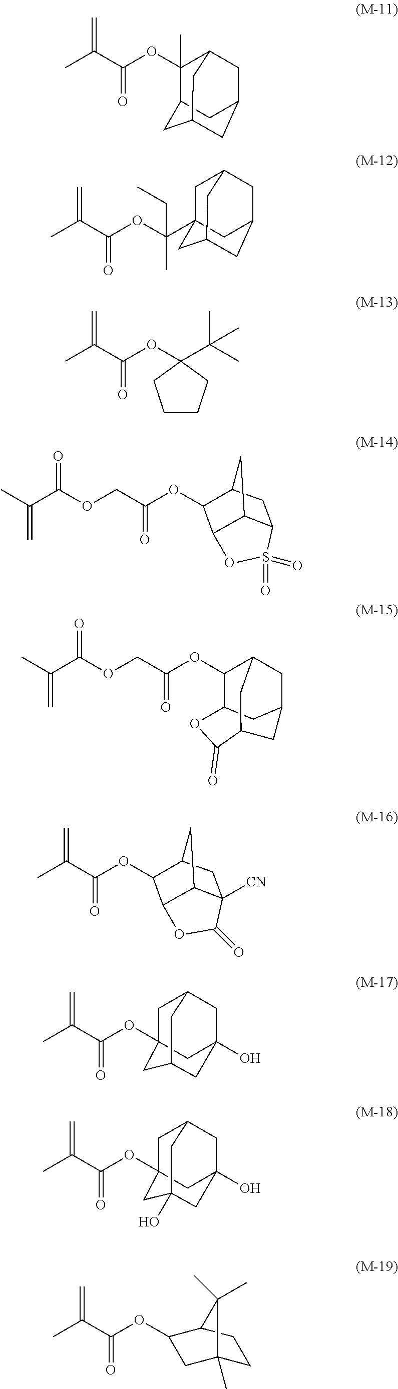

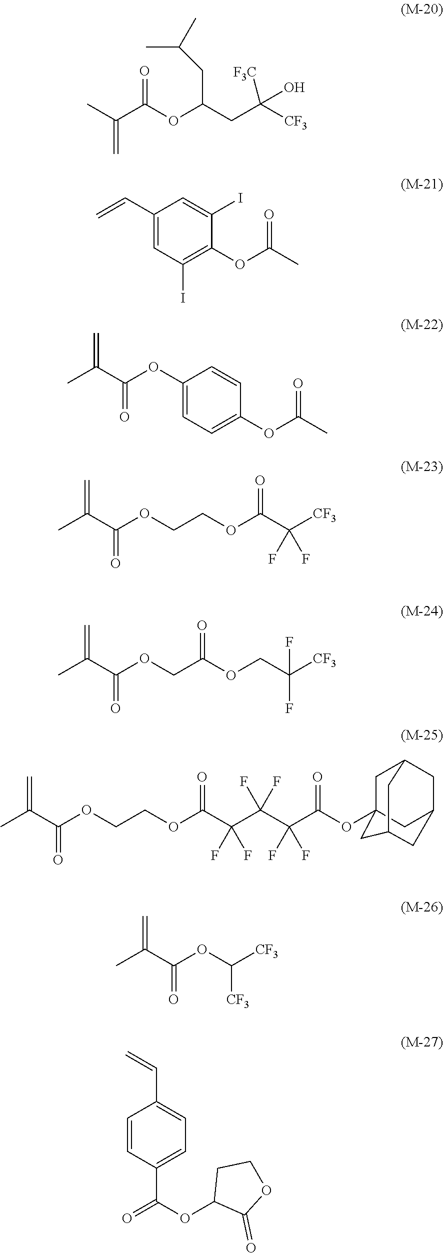

### Synthesis Example 11

### (Synthesis of Polymer (A-1))

A monomer (M-1), a monomer (M-10), a monomer (M-12), a monomer (M-16), and a monomer (M-17) were dissolved at a molar ratio of 35/40/10/10/5 (mol %) in 2-butanone (200 parts by mass), and MAIB (dimethyl 2,2-azobisisobutyrate) (7 mol % based on 100 mol % in total of the monomers used) was added thereto as an initiator to prepare a monomer solution. 2-butanone (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The polymerization solution cooled was poured into methanol (2,000 parts by mass), and a precipitated white powder was collected by filtration. The white powder separated by filtration was washed with methanol twice, then separated by filtration, and dried at 50° C. for 24 hours to obtain a white powdery polymer (A-1) (yield: 85%). The polymer (A-1) had an Mw of 5,500 and an Mw/Mn of 1.56. As a result of ¹³C-NMR analysis, the content ratios of the structural units derived from (M-1), (M-10), (M-12), (M-16), and (M-17) were 35.1 mol %, 40.3 mol %, 9.2 mol %, 10.7 mol %, and 4.7 mol %, respectively.

### Synthesis Examples 2 to 12 and 101 to 102

### (Synthesis of Polymers (A-2) to (A-12) and (A-101) to (A-102)

Polymers (A-2) to (A-12) and (A-101) to (A-102) were synthesized in the same manner as in Synthesis Example 1 except that monomers of types and blending ratios shown in the following Table 1 were used. The content by percent (mol) of each of the structural units and physical property values (Mw and Mw/Mn) of the resulting polymers are also shown in Table 1. In Table 1, “-” indicates that the corresponding monomer was not used (the same applies to Tables below).

TABLE 1

| | | Monomer that affords | | | Monomer that affords | | | |
|---|---|---|---|---|---|---|---|---|
| | | structural unit (II) | | | structural unit (I) | | | |
| | | | Blending | Content ratio | | Blending | Content ratio | Monomer that affords |
| | Polymer | | ratio | of structural | | ratio | of structural | structural unit (III) |
| | [A] | Type | (mol %) | unit (mol %) | Type | (mol %) | unit (mol %) | Type |
| Synthesis | A-1 | M-10 | 40 | 40.3 | M-1 | 35 | 35.1 | M-16 |
| Example 1 | | M-12 | 10 | 9.2 | | | | |
| Synthesis | A-2 | M-10 | 30 | 30.2 | M-1 | 40 | 40.2 | M-14 |
| Example 2 | | M-11 | 20 | 19.2 | | | | |
| Synthesis | A-3 | M-10 | 40 | 39.5 | M-2 | 40 | 40.9 | — |
| Example 3 | | M-12 | 20 | 19.6 | | | | |
| Synthesis | A4 | M-10 | 40 | 40.5 | M-3 | 50 | 49.2 | — |
| Example 4 | | M-12 | 10 | 10.3 | | | | |
| Synthesis | A-5 | M-10 | 40 | 40.6 | M-5 | 50 | 51.3 | — |
| Example 5 | | M-13 | 10 | 8.1 | | | | |
| Synthesis | A-6 | M-13 | 40 | 40.9 | M-6 | 20 | 20.5 | M-9 |
| Example 6 | | M-12 | 10 | 8.3 | | | | |
| Synthesis | A-7 | M-10 | 50 | 50.2 | M-4 | 35 | 34.0 | M-15 |
| Example 7 | | | | | | | | |
| Synthesis | A-8 | M-10 | 40 | 40.0 | M-7 | 20 | 20.5 | M-14 |
| Example 8 | | M-11 | 10 | 10.1 | | | | |
| Synthesis | A-9 | M-13 | 50 | 50.3 | M-8 | 50 | 49.7 | — |
| Example 9 | | | | | | | | |
| Synthesis | A-10 | M-11 | 40 | 40.2 | M-2 | 60 | 59.8 | — |
| Example 10 | | | | | | | | |
| Synthesis | A-11 | M-11 | 40 | 39.4 | M-1 | 40 | 40.7 | — |
| Example 11 | | | | | M-5 | 15 | 15.7 | |
| Synthesis | A-12 | M-11 | 40 | 40.5 | — | — | — | M-16 |
| Example 12 | | | | | | | | |
| Synthesis | A-101 | M-13 | 40 | 39.8 | M-1 | 55 | 55.8 | — |
| Example | | | | | | | | |
| 101 | | | | | | | | |
| Synthesis | A-102 | M-13 | 60 | 60.3 | M-1 | 40 | 39.7 | — |
| Example | | | | | | | | |
| 102 | | | | | | | | |

| | | Monomer that affords | | Monomer that affords | | | | |
|---|---|---|---|---|---|---|---|---|
| | | structural unit (III) | | structural unit (IV) or (VIII) | | | | |
| | | Blending | Content ratio | | Blending | Content ratio | | |
| | Polymer | ratio | of structural | | ratio | of structural | | Mw/ |
| | [A] | (mol %) | unit (mol %) | Type | (mol %) | unit (mol %) | Mw | Mn |
| Synthesis | A-1 | 10 | 10.7 | M-17 | 5 | 4.7 | 5500 | 1.56 |
| Example 1 | | | | | | | | |
| Synthesis | A-2 | 10 | 10.4 | — | — | — | 5000 | 1.51 |
| Example 2 | | | | | | | | |
| Synthesis | A-3 | — | — | — | — | — | 4900 | 1.59 |
| Example 3 | | | | | | | | |
| Synthesis | A4 | — | — | — | — | — | 6600 | 1.61 |
| Example 4 | | | | | | | | |
| Synthesis | A-5 | — | — | — | — | — | 6100 | 1.44 |
| Example 5 | | | | | | | | |
| Synthesis | A-6 | 20 | 20.3 | M-19 | 10 | 10.0 | 1200 | 1.51 |
| Example 6 | | | | | | | | |
| Synthesis | A-7 | 10 | 10.6 | M-17 | 5 | 5.2 | 21200 | 1.55 |
| Example 7 | | | | | | | | |
| Synthesis | A-8 | 20 | 20.2 | M-18 | 10 | 9.2 | 5800 | 1.62 |
| Example 8 | | | | | | | | |
| Synthesis | A-9 | — | — | — | — | — | 6300 | 1.51 |
| Example 9 | | | | | | | | |
| Synthesis | A-10 | — | — | — | — | — | 6700 | 1.50 |
| Example 10 | | | | | | | | |
| Synthesis | A-11 | — | — | M-18 | 5 | 4.2 | 6200 | 1.49 |
| Example 11 | | | | | | | | |
| Synthesis | A-12 | 60 | 59.5 | — | — | — | 5900 | 1.61 |
| Example 12 | | | | | | | | |
| Synthesis | A-101 | — | — | M-17 | 5 | 4.4 | 4900 | 1.55 |
| Example | | | | | | | | |
| 101 | | | | | | | | |
| Synthesis | A-102 | — | — | — | — | — | 8200 | 1.67 |
| Example | | | | | | | | |
| 102 | | | | | | | | |

### (Synthesis of Polymer (A-13))

The monomer (M-1), the monomer (M-10), and a monomer (M-21) were dissolved at a molar ratio of 5/50/45 (mol %) in 1-methoxy-2-propanol (200 parts by mass), and MAIB (dimethyl 2,2-azobisisobutyrate) (7 mol %) was added thereto as an initiator to prepare a monomer solution. 1-methoxy-2-propanol (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The cooled polymerization solution was poured into hexane (2,000 parts by mass), and a precipitated white powder was collected by filtration. The white powder separated by filtration was washed with hexane twice, then separated by filtration, and dissolved in 1-methoxy-2-propanol (300 parts by mass). Next, methanol (500 parts by mass), triethylamine (50 parts by mass) and ultrapure water (10 parts by mass) were added, and a hydrolysis reaction was performed at 70° C. for 6 hours with stirring. After the completion of the reaction, the remaining solvent was distilled off. The resulting solid was dissolved in acetone (100 parts by mass), and the solution was added dropwise to water (500 parts by mass) to solidify a polymer. The resulting solid was separated by filtration, and dried at 50° C. for 13 hours to obtain a white powdery polymer (A-13) (yield: 81%). The polymer (A-13) had an Mw of 5,500 and an Mw/Mn of 1.62. As a result of ¹³C-NMR analysis, the contents by percent of the structural units derived from (M-1), (M-10) and (M-21) were respectively 5.2 mol %, 50.2 mol % and 44.6 mol %.

### Synthesis Examples 14 to 17, and 103

### (Synthesis of Polymers (A-14) to (A-17) and (A-103))

Polymers (A-14) to (A-17) and (A-103) were synthesized in the same manner as in Synthesis Example 13 except that monomers of types and blending ratios shown in the following Table 2 were used. For the monomer that gives the structural unit (IV), in the polymer, the disappearance of the peak of the carbonyl group on the acetyl group was confirmed by measurement of ¹³C-NMR, and substantially all the alkali-dissociable groups were hydrolyzed to phenolic hydroxyl groups. The content ratio (mol %) and physical property values (Mw and Mw/Mn) of each of the structural units of the resulting polymers are also shown in the following Table 2.

TABLE 2

| | | Monomer that affords structural | | | Monomer that affords | | | |
|---|---|---|---|---|---|---|---|---|
| | | unit (II) | | | structural unit (I) | | | |
| | | | Blending | Content ratio | | Blending | Content ratio | Monomer that affords structural |
| | Polymer | | ratio | of structural | | ratio | of structural | unit (IV) |
| | [A] | Type | (mol %) | unit (mol %) | Type | (mol %) | unit (mol %) | Type |
| Synthesis | A-13 | M-10 | 50 | 50.2 | M-1 | 5 | 5.2 | — |
| Example 13 | | | | | | | | |
| Synthesis | A-14 | M-12 | 40 | 40.6 | M-2 | 10 | 10.6 | M-17 |
| Example 14 | | | | | | | | |
| Synthesis | A-15 | M-11 | 50 | 48.1 | M-4 | 5 | 4.9 | M-20 |
| Example 15 | | | | | | | | |
| Synthesis | A-16 | M-13 | 40 | 41.2 | M-6 | 20 | 20.2 | M-20 |
| Example 16 | | | | | | | | |
| Synthesis | A-17 | M-10 | 50 | 53.2 | — | — | — | — |
| Example 17 | | | | | | | | |
| Synthesis | A-103 | M-13 | 60 | 58.4 | M-27 | 10 | 8.7 | — |
| Example | | | | | | | | |
| 103 | | | | | | | | |

| | | Monomer that affords structural | | Monomer that affords | | | | |
|---|---|---|---|---|---|---|---|---|
| | | unit (IV) | | structural unit (V) | | | | |
| | | Blending | Content ratio | | Blending | Content ratio | | |
| | Polymer | ratio | of structural | | ratio | of structural | | Mw/ |
| | [A] | (mol %) | unit (mol %) | Type | (mol %) | unit (mol %) | Mw | Mn |
| Synthesis | A-13 | — | — | M-21 | 45 | 44.6 | 5500 | 1.62 |
| Example 13 | | | | | | | | |
| Synthesis | A-14 | 10 | 10.1 | M-22 | 40 | 38.7 | 5600 | 1.55 |
| Example 14 | | | | | | | | |
| Synthesis | A-15 | 15 | 15.3 | M-21 | 30 | 31.7 | 5100 | 1.59 |
| Example 15 | | | | | | | | |
| Synthesis | A-16 | 10 | 10.2 | M-22 | 30 | 28.4 | 6100 | 1.50 |
| Example 16 | | | | | | | | |
| Synthesis | A-17 | — | — | M-21 | 50 | 46.8 | 6100 | 1.50 |
| Example 17 | | | | | | | | |
| Synthesis | A-103 | — | — | M-21 | 30 | 32.9 | 7800 | 1.48 |
| Example | | | | | | | | |
| 103 | | | | | | | | |

### Synthesis Example 18

### [Synthesis of High Fluorine-Containing Polymer (F-1)]

Monomers (M-10) and (M-23) were dissolved at a molar ratio of 20/80 (mol %) in 2-butanone (200 parts by mass), and AIBN (4 mol %) was added thereto as an initiator to prepare a monomer solution. 2-butanone (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The solvent was replaced with acetonitrile (400 parts by mass). Hexane (100 parts by mass) was then added, followed by stirring, and an acetonitrile layer was collected. The operation was repeated three times. By replacing the solvent with propylene glycol monomethyl ether acetate, a solution of a high fluorine-containing polymer (F-1) was obtained (yield: 75%). The high fluorine-content polymer (F-1) had an Mw of 6,200 and an Mw/Mn of 1.77. As a result of ¹³C-NMR analysis, the contents by percent of the structural units derived from (M-10) and (M-23) were 19.5 mol % and 80.5 mol %, respectively.

### Synthesis Examples 19 to 22, and 104

### (Synthesis of High Fluorine-Containing Polymers (F-2) to (F-5), and (F-101) )

High fluorine-containing polymers (F-2) to (F-5), and (F-101) were synthesized in the same manner as in Synthesis Example 18 except that monomers of types and blending ratios shown in Table 3 were used. The content by percent (mol %) of each of the structural units and physical property values (Mw and Mw/Mn) of the resulting high fluorine-containing polymers are also shown in Table 3.

TABLE 3
Monomer that affords structural Monomer that affords structural

| | | Monomer that affords structural | | | Monomer that affords structural | | | |
|---|---|---|---|---|---|---|---|---|
| | High | unit (VII) | | | unit (II) | | | |
| | fluorine- | | Blending | Content ratio | | Blending | Content ratio | Monomer that affords |
| | content | | ratio | of structural | | ratio | of structural | structural unit (IV) |
| | polymer | Type | (mol %) | unit (mol %) | Type | (mol %) | unit (mol %) | Type |
| Synthesis | F-1 | M-23 | 80 | 80.5 | M-10 | 20 | 19.5 | — |
| Example 18 | | | | | | | | |
| Synthesis | F-2 | M-24 | 80 | 80.9 | M-13 | 20 | 19.1 | — |
| Example 19 | | | | | | | | |
| Synthesis | F-3 | M-25 | 60 | 62.3 | — | — | — | — |
| Example 20 | | | | | | | | |
| Synthesis | F-4 | M-25 | 60 | 60.2 | M-11 | 20 | 19.4 | M-18 |
| Example 21 | | | | | | | | |
| Synthesis | F-5 | M-23 | 60 | 60.0 | M-12 | 10 | 10.1 | M-20 |
| Example 22 | | | | | | | | |
| Synthesis | F-101 | M-24 | 50 | 60.2 | M-13 | 40 | 19.4 | — |
| Example | | | | | | | | |
| 104 | | M-26 | 10 | 60.0 | | | | |

| | | Monomer that affords | | Monomer that affords | | | | |
|---|---|---|---|---|---|---|---|---|
| | High | structural unit (IV) | | structural unit (VIII) | | | | |
| | fluorine- | Blending | Content ratio | | Blending | Content ratio | | |
| | content | ratio | of structural | | ratio | of structural | | Mw/ |
| | polymer | (mol %) | unit (mol %) | Type | (mol %) | unit (mol %) | Mw | Mn |
| Synthesis | F-1 | — | — | — | — | — | 6200 | 1.77 |
| Example 18 | | | | | | | | |
| Synthesis | F-2 | — | — | — | — | — | 7100 | 1.82 |
| Example 19 | | | | | | | | |
| Synthesis | F-3 | — | — | M-19 | 40 | 37.7 | 6900 | 1.91 |
| Example 20 | | | | | | | | |
| Synthesis | F-4 | 20 | 20.4 | — | — | — | 7300 | 1.88 |
| Example 21 | | | | | | | | |
| Synthesis | F-5 | 30 | 29.9 | — | — | — | 6700 | 1.87 |
| Example 22 | | | | | | | | |
| Synthesis | F-101 | — | — | — | — | — | 5500 | 1.78 |
| Example | | | | | | | | |
| 104 | | | | | | | | |

### <Synthesis of Onium Salt Compound B>

### Synthesis Example 21

### (Synthesis of Onium Salt Compound (B-1))

A onium salt compound (B-1) as a radiation-sensitive acid generator was synthesized according to the following synthesis scheme.

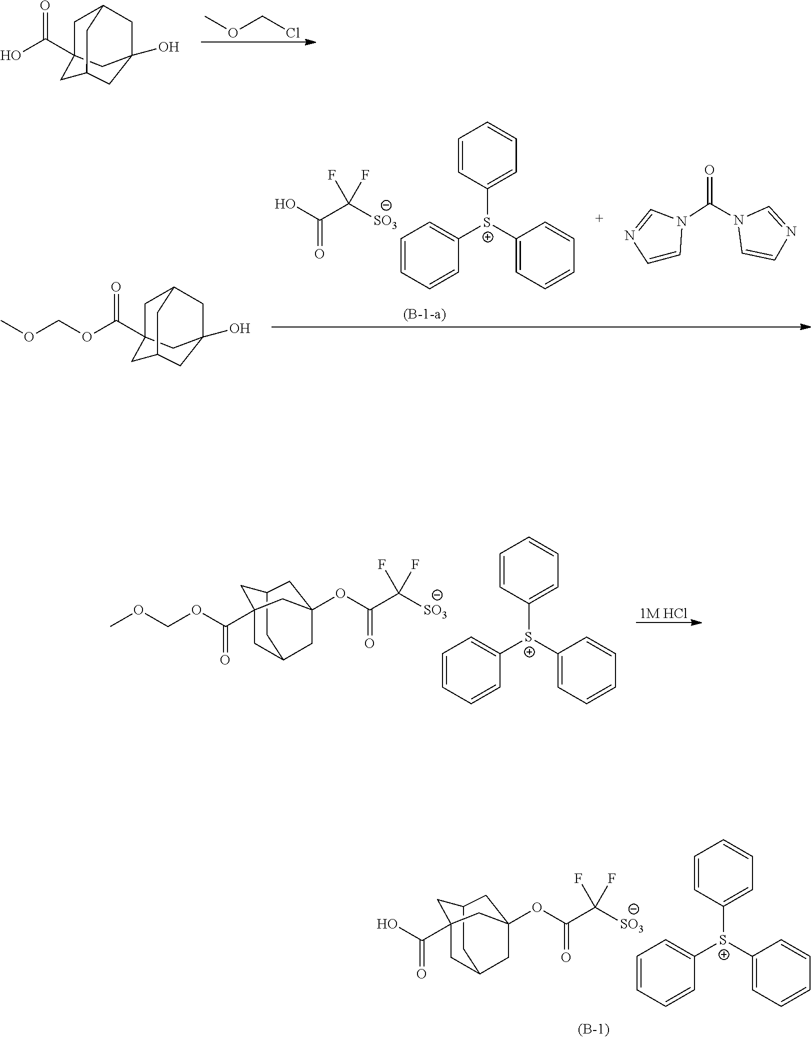

A reaction vessel was charged with 20.0 mmol of 3-hydroxy-1-adamantanecarboxylic acid, 20.0 mmol of chloromethyl methyl ether, 20.0 mmol of triethylamine, and 50 g of dichloromethane, and the mixture was stirred at room temperature for 13 hours. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and purification was performed by column chromatography to obtain a carboxylic acid protector in a good yield.

To the carboxylic acid protector, 20.0 mmol of an onium salt compound (B-1-a) represented by the formula (B-1-a), 20.0 mmol of 1,1-carbonyldiimidazole, 20.0 mmol of pyridine, and 50 g of acetonitrile were added, and the mixture was stirred at 50° C. for 10 hours. Thereafter, water was added to stop the reaction, dichloromethane was then added thereto to perform extraction, and the organic layer was separated. The resulting organic layer was washed with a saturated aqueous solution of sodium chloride and then with water. After drying over sodium sulfate, the solvent was distilled off, and purification was performed by column chromatography to obtain a carboxylic acid protector onium salt in a good yield.

To the carboxylic acid protector onium salt, 50 g of 1-M hydrochloric acid and 50 g of acetonitrile were added, and the mixture was stirred at room temperature for 24 hours. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and purification was performed by column chromatography to obtain an onium salt compound (B-1) represented by the formula (B-1) in a good yield.

### Synthesis Examples 24 to 31

### (Synthesis of Onium Salt Compounds (B-2) to (B-9), (B-17), and (B-18))

Onium salt compounds (B-2) to (B-9), (B-17), and (B-18) represented by the following formulas (B-2) to (B-9), (B-17), and (B-18) were synthesized in the same manner as in Synthesis Example 23 except that the raw materials and the precursor were appropriately changed.

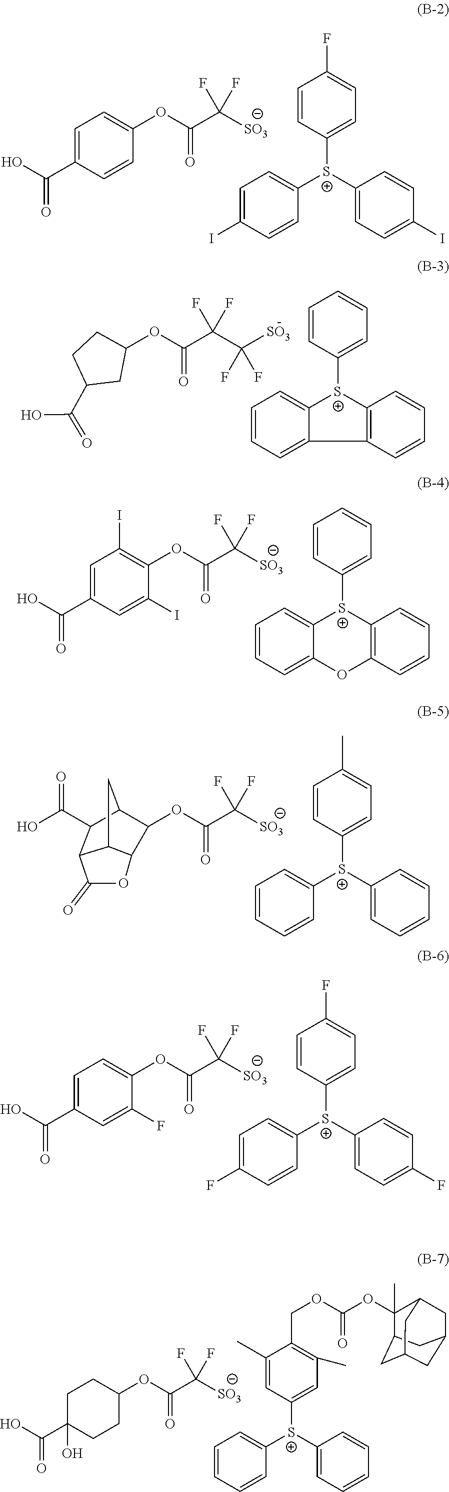

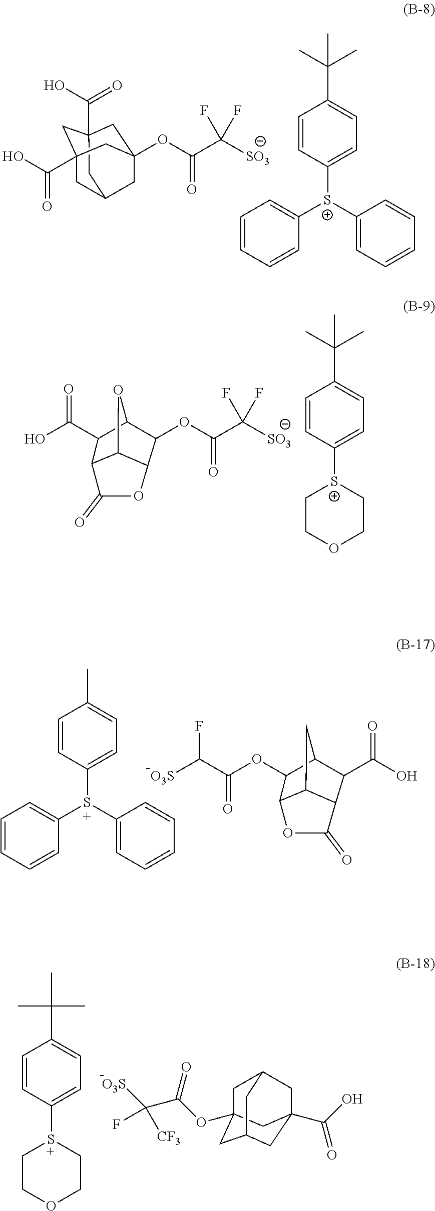

### Synthesis Example 32

### (Synthesis of Onium Salt Compound (B-10))

An onium salt compound (B-10) was synthesized in accordance with the synthesis scheme below.

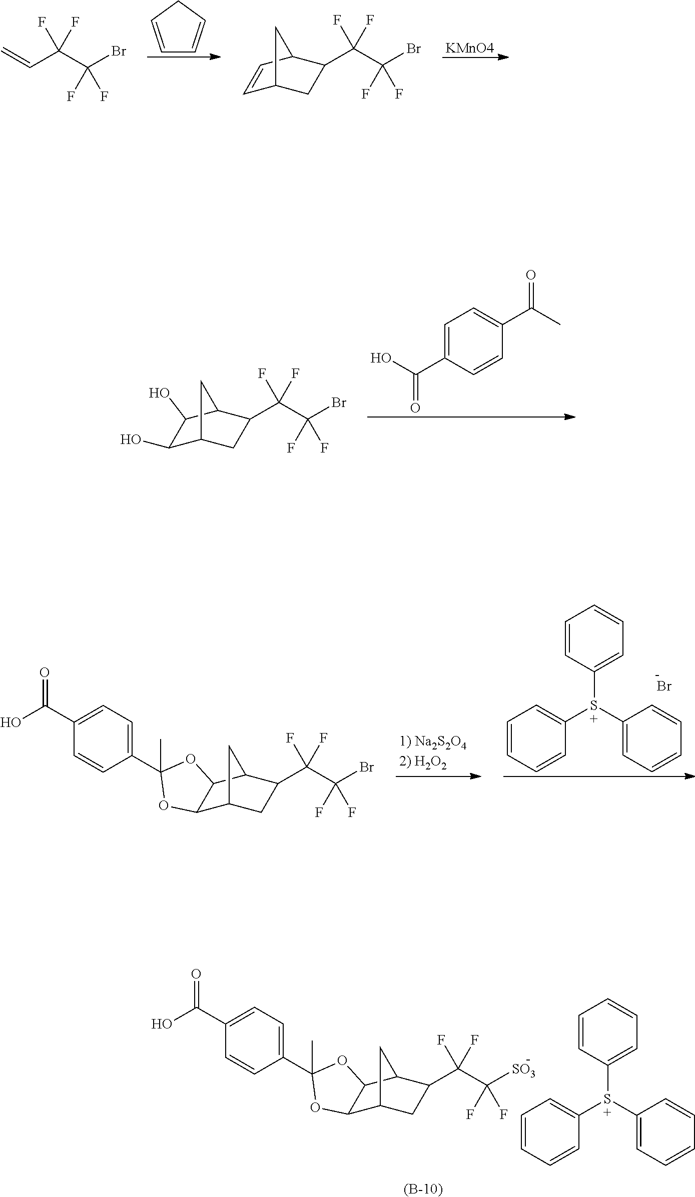

In a reaction vessel, 20.0 mmol of cyclopentadiene and 50 g of dichloromethane were added to 20.0 mmol of 4-bromo-3,3,4,4-tetrafluoro-1-butene, and the mixture was stirred at room temperature for 3 hours. Thereafter, water was added to dilute the mixture, followed by addition of dichloromethane and extraction, and then the organic layer was separated. The resulting organic layer was washed with a saturated aqueous sodium chloride solution and then with water. After drying over sodium sulfate, the solvent was distilled off, and the residue was purified by column chromatography, affording an olefin form in a good yield.

To the olefin form were added 40.0 mmol of potassium permanganate and 50 g of acetonitrile, and the mixture was stirred at 50° C. for 10 hours. Thereafter, a saturated aqueous sodium thiosulfate solution was added to stop the reaction, followed by addition of ethyl acetate and extraction, and then the organic layer was separated. The resulting organic layer was washed with a saturated aqueous sodium chloride solution and then with water. After drying over sodium sulfate, the solvent was distilled off, and the residue was purified by column chromatography, affording a diol form in a good yield.

To the diol form were added 20.0 mmol of 4-acetylbenzoic acid, 2.00 mmol of sulfuric acid, and 50 g of dichloromethane, and the mixture stirred at room temperature for 24 hours. Thereafter, water was added to dilute the mixture, followed by addition of ethyl acetate and extraction, and then the organic layer was separated. The resulting organic layer was washed with a saturated aqueous sodium chloride solution and then with water. After drying over sodium sulfate, a solvent was distilled off, and the residue was purified by column chromatography, affording an acetal form in a good yield.

A mixed liquid of acetonitrile and water (1:1 (mass ratio)) was added to the acetal form to form a 1 M solution. Then, 40.0 mmol of sodium dithionite and 60.0 mmol of sodium hydrogen carbonate were added, and the mixture was reacted at 70° C. for 4 hours. After extraction with acetonitrile and distillation of the solvent, a mixed liquid of acetonitrile and water (3:1 (mass ratio)) was added to form a 0.5 M solution. 60.0 mmol of hydrogen peroxide water and 2.00 mmol of sodium tungstate were added, and the mixture was heated and stirred at 50° C. for 12 hours. The mixture was extracted with acetonitrile, and the solvent was distilled off, affording a sodium sulfonate salt compound. 20.0 mmol of triphenylsulfonium bromide was added to the sodium sulfonate salt compound, and a mixed liquid of water and dichloromethane (1:3 (mass ratio)) was added to form a 0.5 M solution. The solution was vigorously stirred at room temperature for 3 hours. Thereafter, dichloromethane was added thereto, followed by extraction, and then the organic layer was separated. The resulting organic layer was dried over sodium sulfate, the solvent was then distilled off, and the residue was purified by column chromatography, affording an onium salt compound (B-10) represented by the formula (B-1) in a good yield.

### Synthesis Examples 33 to 36

### (Synthesis of Onium Salt Compounds (B-11) to (B-14))

Onium salt compounds (B-11) to (B-14) represented by the following formulas (B-11) to (B-14) were synthesized in the same manner as in Synthesis Example 23 except that the raw materials and the precursor were appropriately changed.

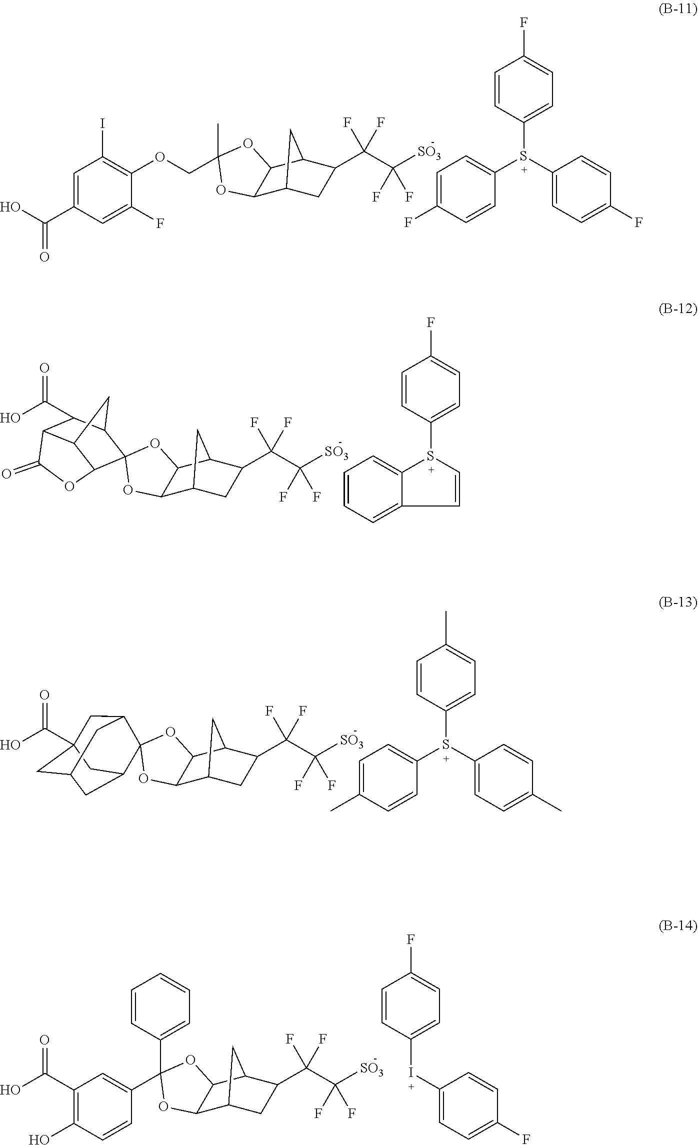

### Synthesis Example 37

### (Synthesis of Onium Salt Compound (B-i))

An onium salt compound (B-a) was synthesized in accordance with the synthesis scheme below.

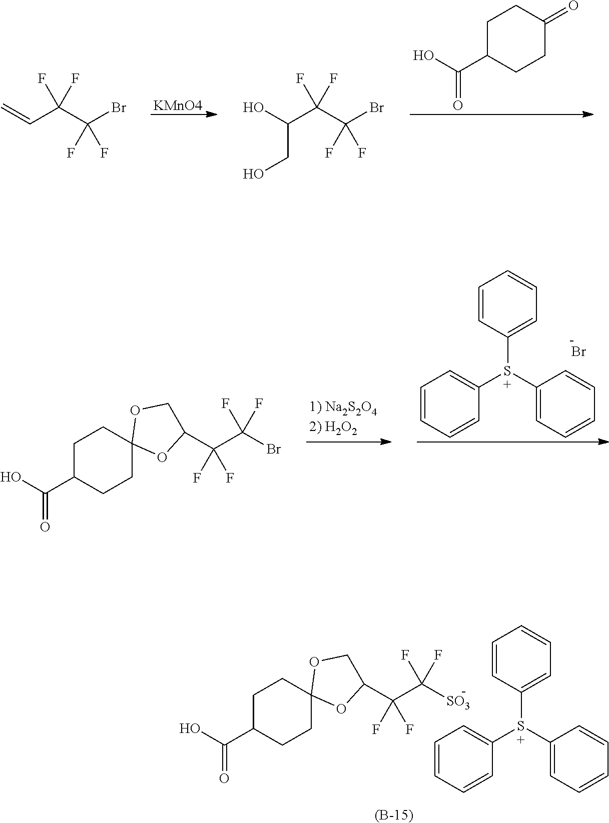

To a reaction vessel were added 20.0 mmol of 4-bromo-3,3,4,4-tetrafluoro-1-butene, 40.0 mmol of potassium permanganate, and 50 g of acetonitrile, and the mixture was stirred at 50° C. for 10 hours. Thereafter, a saturated aqueous sodium thiosulfate solution was added to stop the reaction, followed by addition of ethyl acetate and extraction, and then the organic layer was separated. The resulting organic layer was washed with a saturated aqueous solution of sodium chloride and then with water. After drying over sodium sulfate, the solvent was distilled off, and the residue was purified by column chromatography, affording a diol form in a good yield.

To the diol form were added 20.0 mmol of cyclohexanone-4-carboxylic acid, 2.00 mmol of sulfuric acid, and 50 g of dichloromethane, and the mixture was stirred at room temperature for 24 hours. Thereafter, water was added to dilute the mixture, followed by addition of ethyl acetate and extraction, and then the organic layer was separated. The resulting organic layer was washed with a saturated aqueous sodium chloride solution and then with water. After drying over sodium sulfate, a solvent was distilled off, and purification was performed by column chromatography to obtain an acetal form in good yield.

A mixed liquid of acetonitrile and water (1:1 (mass ratio)) was added to the acetal form to form a 1 M solution. Then, 40.0 mmol of sodium dithionite and 60.0 mmol of sodium hydrogen carbonate were added, and the mixture was reacted at 70° C. for 4 hours. After extraction with acetonitrile and distillation of the solvent, a mixed liquid of acetonitrile and water (3:1 (mass ratio)) was added to form a 0.5 M solution. 60.0 mmol of hydrogen peroxide water and 2.00 mmol of sodium tungstate were added, and the mixture was heated and stirred at 50° C. for 12 hours. The mixture was extracted with acetonitrile, and the solvent was distilled off, affording a sodium sulfonate salt compound. 20.0 mmol of triphenylsulfonium bromide was added to the sodium sulfonate salt compound, and a mixed liquid of water and dichloromethane (1:3 (mass ratio)) was added to form a 0.5 M solution. The solution was vigorously stirred at room temperature for 3 hours. Thereafter, dichloromethane was added thereto, followed by extraction, and then the organic layer was separated. After drying the resulting organic layer over sodium sulfate, the solvent was distilled off, and purification was performed by column chromatography, affording an onium salt compound represented by the formula (B-15) in good yield.

### Synthesis Example 38

### (Synthesis of Onium Salt Compound (B-16))

An onium salt compound (B-16) represented by the following formula (B-16) was synthesized in the same manner as in Synthesis Example 37 except that the raw materials and the precursor were appropriately changed.

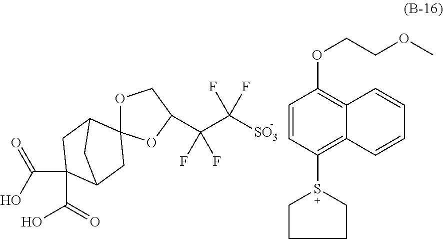

### Example B1

### (Synthesis of Onium Salt Compound (B-19))

An onium salt compound (B-19) was synthesized in accordance with the synthesis scheme below.

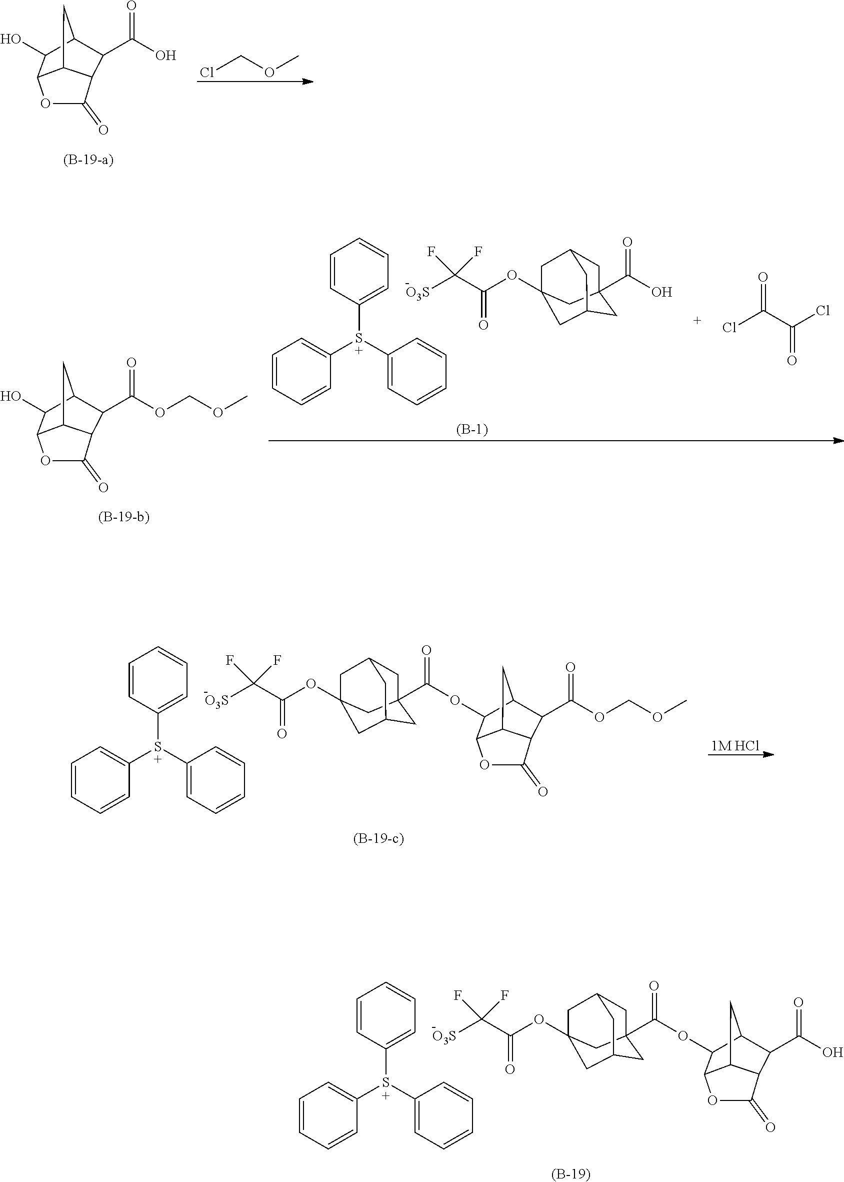

A reaction vessel was charged with 20.0 mmol of a compound (B-19-a), 20.0 mmol of chloromethyl methyl ether, 20.0 mmol of triethylamine, and 50 g of dichloromethane, and the mixture was stirred at room temperature for 13 hours. Thereafter, water was added to dilute the mixture, followed by addition of dichloromethane and extraction, and then the organic layer was separated. The resulting organic layer was washed with a saturated aqueous solution of sodium chloride and then with water. After the organic layer was dried over sodium sulfate, the solvent was distilled off, and the residue was purified by column chromatography, affording a compound (B-19-b) in a good yield.

A reaction vessel was charged with 20.0 mmol of the onium salt compound (B-1) represented by the formula (B-1), 20.0 mmol of oxalyl chloride, and 50 g of acetonitrile, and the mixture was stirred at 50° C. for 2 hours. Then, 30.0 mmol of the compound (B-19-b), 30.0 mmol of pyridine, and 3.0 mmol of 4-dimethylaminopyridine were added, and the mixture was stirred at 100° C. for 10 hours. Thereafter, water was added to stop the reaction, dichloromethane was then added thereto to perform extraction, and the organic layer was separated. The resulting organic layer was washed with a saturated aqueous solution of sodium chloride and then with water. After the organic layer was dried over sodium sulfate, the solvent was distilled off, and the residue was purified by column chromatography, affording an onium salt compound (B-19-c) in a good yield.

To the onium salt compound (B-19-c) represented by the formula (B-19-c), 50 g of 1-M hydrochloric acid and 50 g of acetonitrile were added, and the mixture was stirred at 100° C. for 3 hours. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and purification was performed by column chromatography to obtain an onium salt compound (B-19) represented by the formula (B-19) in a good yield.

### Examples B2 to B5

### (Synthesis of Onium Salt Compounds (B-20) to (B-23))

Onium salt compounds (B-20) to (B-23) represented by the following formulas (B-20) to (B-23) were synthesized in the same manner as in Example B1 except that the raw materials and the precursor were appropriately changed.

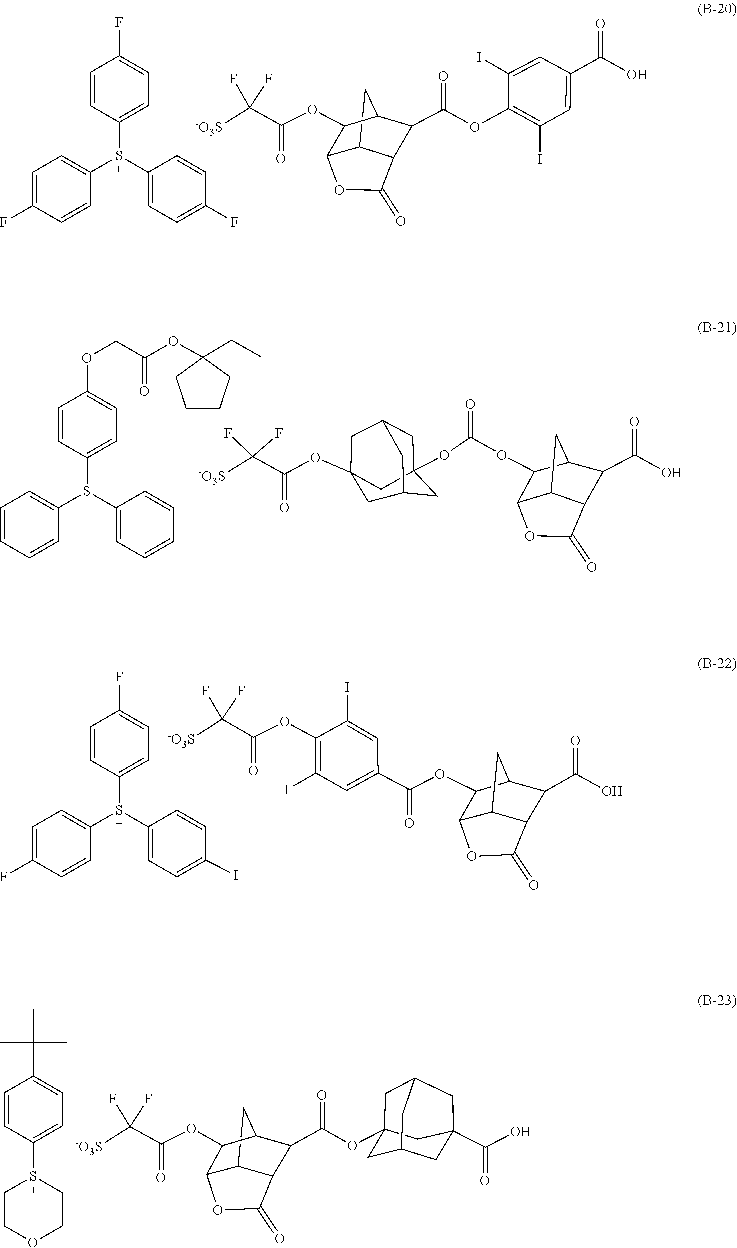

The following compounds were used as components other than the synthesized components.

[Radiation-Sensitive Acid Generators Other than Onium Salt Compounds (B-1) to (B-23)]

b-1 to b-15: Compounds represented by the following formulas (b-1) to (b-15) (hereinafter, the compounds represented by the formulas (b-1) to (b-15) may be described as “compound (b-1)” to “compound (b-15)”, respectively).

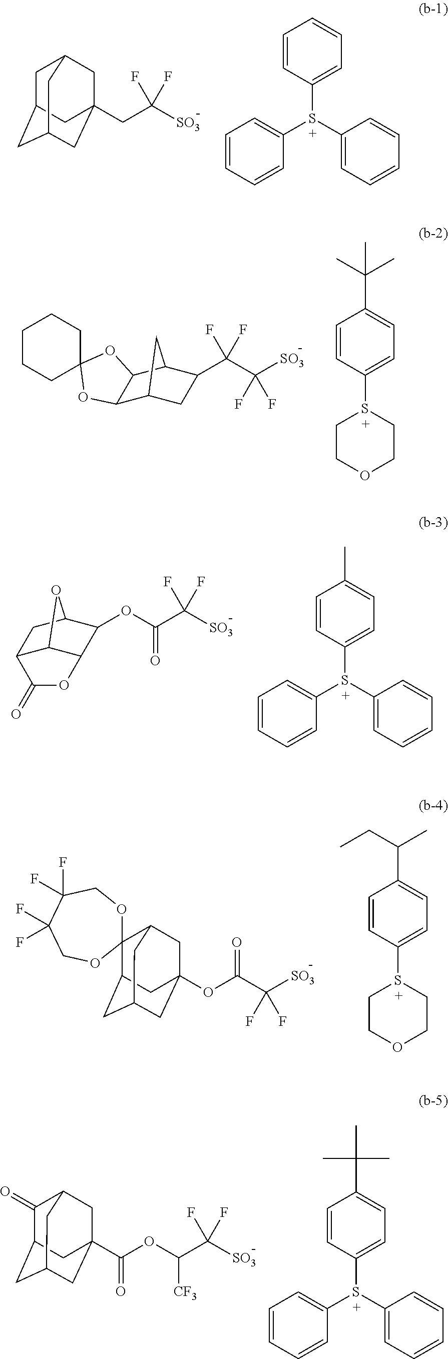

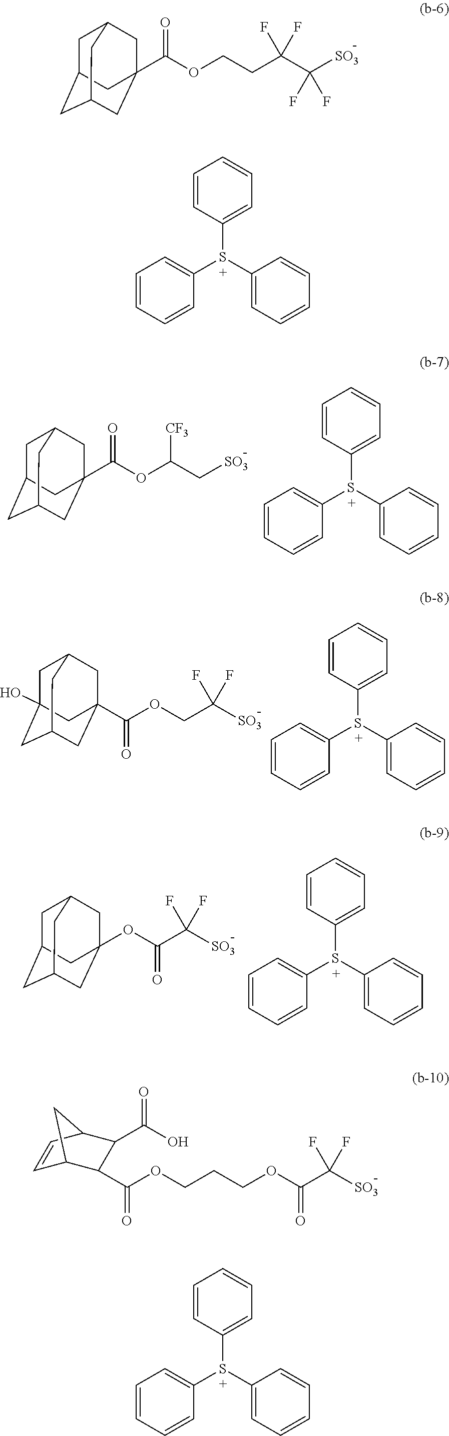

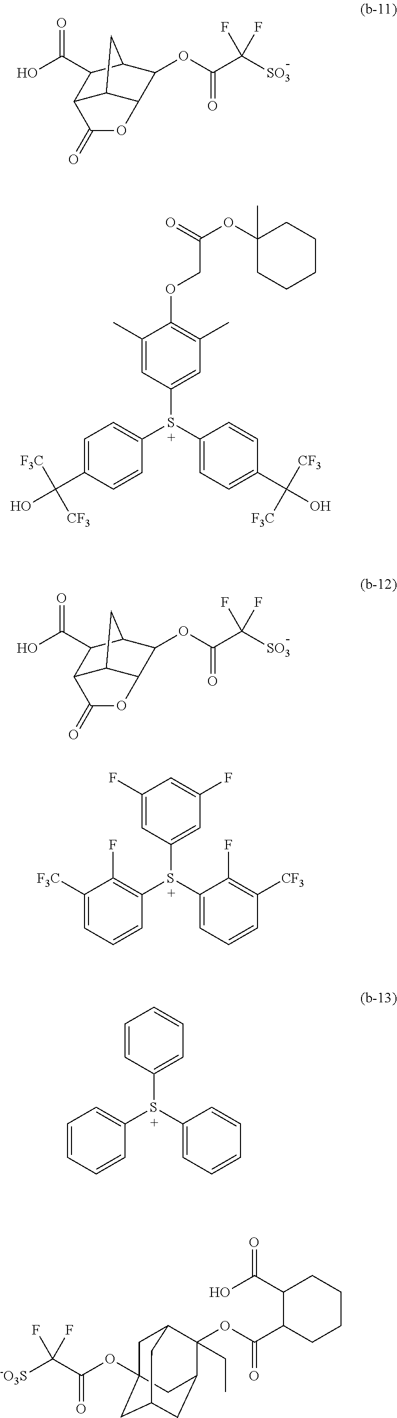

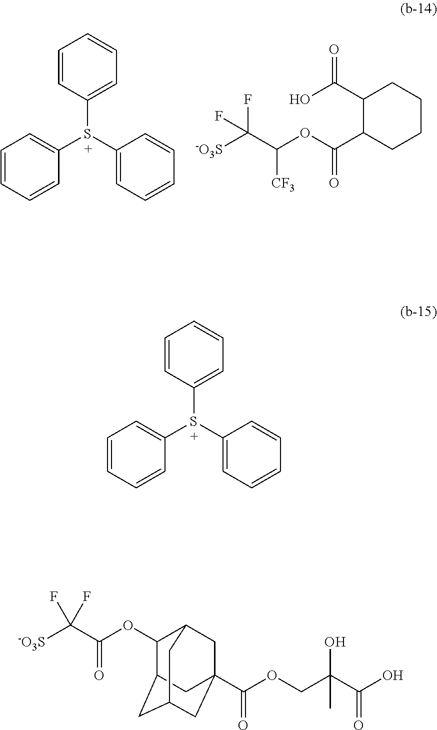

### [Acid Diffusion Controlling Agent [D]]

D-1 to D-12: Compounds represented by formulas (D-1) to (D-12) below.

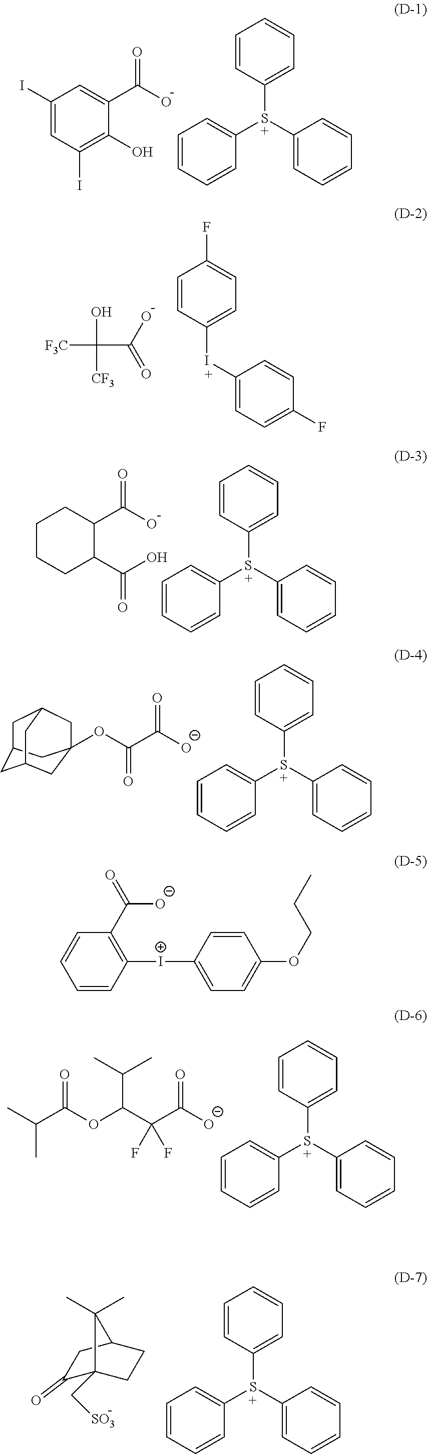

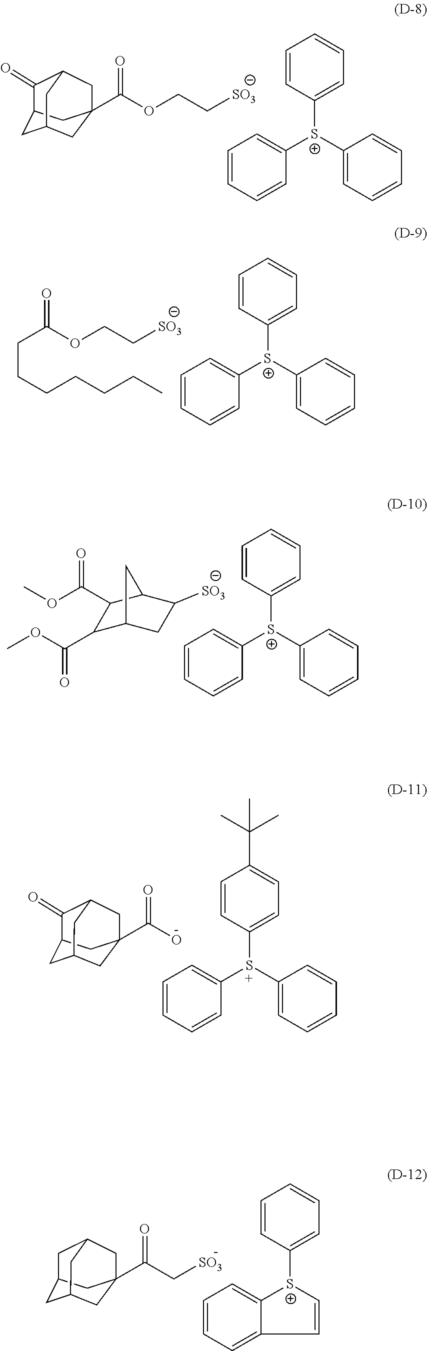

### [Solvent [E]]

E-1: Propylene glycol monomethyl ether acetate
E-2: Propylene glycol monomethyl ether
E-3: Y-Butyrolactone
E-4: Ethyl lactate
E-5: Diacetone alcohol
E-6: Methyl 2-hydroxyisobutyrate

### [Preparation of Positive Radiation-Sensitive Composition for ArF Immersion Exposure]

### Example 1

After mixing 100 parts by mass of (A-1) as the polymer [A], 10.0 parts by mass of (B-1) as the onium salt compound [B], 4.0 parts by mass of (D-1) as the acid diffusion controlling agent [D], 5.0 parts by mass (solid content) of (F-1) as the high fluorine-content polymer [F], and 3,400 parts by mass of a mixed solvent of (E-1)/(E-2)/(E-3) as the solvent [E], the mixture was filtered through a membrane filter having a pore diameter of 0.2 μm to prepare a radiation-sensitive composition (J-1).

### Examples 2 to 47 and 101 to 110 and Comparative Examples 1 to and 21 to 25

Radiation-sensitive compositions (J-2) to (J-47), (J-101) to (J-110), (CJ-1) to (CJ-5), and (CJ-21) to (CJ-25) were prepared in the same manner as in Example 1 except that the respective components of the types and contents shown in Table 4 below were used.

TABLE 4

| | | | | | | Acid diffusion | | | | | |
|---|---|---|---|---|---|---|---|---|---|---|---|
| | | | | Onium salt | | controlling agent | | High fluorine- | | | |
| | | Polymer [A] | | compound [B] | | [D] | | content polymer [F] | | | |
| | Radiation- | | Content | | Content | | Content | | Content | Solvent [E] | |
| | sensitive | | (parts by | | (parts by | | (parts by | | (parts by | | Content |
| | composition | Type | mass) | Type | mass) | Type | mass) | Type | mass) | Type | (parts by mass) |
| Example 1 | J-1 | A-1 | 100 | B-1 | 10.0 | D-1 | 4.0 | F-1 | 5.0 | E-1/E-2/E-3 | 1600/1600/200 |
| Example 2 | J-2 | A-2 | 100 | B-1 | 10.0 | D-1 | 4.0 | F-1 | 5.0 | E-1/E-2/E-3 | 1600/1600/200 |
| Example 3 | J-3 | A-3 | 100 | B-1 | 10.0 | D-1 | 4.0 | F-1 | 5.0 | E-1/E-2/E-3 | 1600/1600/200 |
| Example 4 | J-4 | A-4 | 100 | B-1 | 10.0 | D-1 | 4.0 | F-1 | 5.0 | E-1/E-2/E-3 | 1600/1600/200 |
| Example 5 | J-5 | A-5 | 100 | B-1 | 10.0 | D-1 | 4.0 | F-1 | 5.0 | E-1/E-2/E-3 | 1600/1600/200 |
| Example 6 | J-6 | A-6 | 100 | B-1 | 10.0 | D-1 | 4.0 | F-1 | 5.0 | E-1/E-2/E-3 | 1600/1600/200 |
| Example 7 | J-7 | A-7 | 100 | B-1 | 10.0 | D-1 | 4.0 | F- | 5.0 | E-1/E-2/E-3 | 1600/1600/200 |
| Example 8 | J-8 | A-8 | 100 | B-1 | 10.0 | D-1 | 4.0 | F-1 | 5.0 | E-1/E-2/E-3 | 1600/1600/200 |
| Example 9 | J-9 | A-9 | 100 | B-1 | 10.0 | D-1 | 4.0 | F-1 | 5.0 | E-1/E-2/E-3 | 1600/1600/200 |
| Example 10 | J-10 | A-10 | 100 | B-1 | 10.0 | D-1 | 4.0 | F-1 | 5.0 | E-1/E-2/E-3 | 1600/1600/200 |
| Example 11 | J-11 | A-11 | 100 | B-1 | 10.0 | D-1 | 4.0 | F-1 | 5.0 | E-1/E-2/E-3 | 1600/1600/200 |
| Example 101 | J-101 | A-101 | 100 | B-1 | 10.0 | D-1 | 4.0 | F-1 | 5.0 | E-1/E-2/E-3 | 1600/1600/200 |
| Example 102 | J-102 | A-102 | 100 | B-1 | 10.0 | D-1 | 4.0 | F-1 | 5.0 | E-1/E-2/E-3 | 1600/1600/200 |
| Example 12 | J-12 | A-1 | 100 | B-1 | 10.0 | D-2 | 4.0 | F-1 | 5.0 | E-1/E-2/E-3 | 1600/1600/200 |
| Example 13 | J-13 | A-1 | 100 | B-1 | 10.0 | D-3 | 4.0 | F-1 | 5.0 | E-1/E-2/E-3 | 1600/1600/200 |
| Example 14 | J-14 | A-1 | 100 | B-1 | 10.0 | D-4 | 4.0 | F-1 | 5.0 | E-1/E-2/E-3 | 1600/1600/200 |
| Example 15 | J-15 | A-1 | 100 | B-1 | 10.0 | D-5 | 4.0 | F-1 | 5.0 | E-1/E-2/E-3 | 1600/1600/200 |
| Example 16 | J-16 | A-1 | 100 | B-1 | 10.0 | D-6 | 4.0 | F-1 | 5.0 | E-1/E-2/E-3 | 1600/1600/200 |
| Example 17 | J-17 | A-1 | 100 | B-1 | 10.0 | D-7 | 4.0 | F-1 | 5.0 | E-1/E-2/E-3 | 1600/1600/200 |
| Example 18 | J-18 | A-1 | 100 | B-1 | 10.0 | D-8 | 4.0 | F-1 | 5.0 | E-1/E-2/E-3 | 1600/1600/200 |
| Example 19 | J-19 | A-1 | 100 | B-1 | 10.0 | D-9 | 4.0 | F-1 | 5.0 | E-1/E-2/E-3 | 1600/1600/200 |
| Example 20 | J-20 | A-1 | 100 | B-1 | 10.0 | D-10 | 4.0 | F-1 | 5.0 | E-1/E-2/E-3 | 1600/1600/200 |
| Example 21 | J-21 | A-1 | 100 | B-1 | 10.0 | D-11 | 4.0 | F-1 | 5.0 | E-1/E-2/E-3 | 1600/1600/200 |
| Example 22 | J-22 | A-1 | 100 | B-1 | 10.0 | D-12 | 4.0 | F-1 | 5.0 | E-1/E-2/E-3 | 1600/1600/200 |
| Example 23 | J-23 | A-1 | 100 | B-2 | 10.0 | D-1 | 4.0 | F-1 | 5.0 | E-1/E-2/E-3 | 1600/1600/200 |
| Example 24 | J-24 | A-1 | 100 | B-3 | 10.0 | D-1 | 4.0 | F-1 | 5.0 | E-1/E-2/E-3 | 1600/1600/200 |
| Example 25 | J-25 | A-1 | 100 | B-4 | 10.0 | D-1 | 4.0 | F-1 | 5.0 | E-1/E-2/E-3 | 1600/1600/200 |
| Example 26 | J-26 | A-1 | 100 | B-5 | 10.0 | D-1 | 4.0 | F-1 | 5.0 | E-1/E-2/E-3 | 1600/1600/200 |
| Example 27 | J-27 | A-1 | 100 | B-6 | 10.0 | D-1 | 4.0 | F-1 | 5.0 | E-1/E-2/E-3 | 1600/1600/200 |
| Example 28 | J-28 | A-1 | 100 | B-7 | 10.0 | D-1 | 4.0 | F-1 | 5.0 | E-1/E-2/E-3 | 1600/1600/200 |
| Example 29 | J-29 | A-1 | 100 | B-8 | 10.0 | D-1 | 4.0 | F-1 | 5.0 | E-1/E-2/E-3 | 1600/1600/200 |
| Example 30 | J-30 | A-1 | 100 | B-9 | 10.0 | D-1 | 4.0 | F-1 | 5.0 | E-1/E-2/E-3 | 1600/1600/200 |
| Example 31 | J-31 | A-1 | 100 | B-10 | 10.0 | D-1 | 4.0 | F-1 | 5.0 | E-1/E-2/E-3 | 1600/1600/200 |
| Example 32 | J-32 | A-1 | 100 | B-11 | 10.0 | D-1 | 4.0 | F-1 | 5.0 | E-1/E-2/E-3 | 1600/1600/200 |
| Example 33 | J-33 | A-1 | 100 | B-12 | 10.0 | D-1 | 4.0 | F-1 | 5.0 | E-1/E-2/E-3 | 1600/1600/200 |
| Example 34 | J-34 | A-1 | 100 | B-13 | 10.0 | D-1 | 4.0 | F-1 | 5.0 | E-1/E-2/E-3 | 1600/1600/200 |
| Example 35 | J-35 | A-1 | 100 | B-14 | 10.0 | D-1 | 4.0 | F-1 | 5.0 | E-1/E-2/E-3 | 1600/1600/200 |
| Example 36 | J-36 | A-1 | 100 | B-15 | 10.0 | D-1 | 4.0 | F-1 | 5.0 | E-1/E-2/E-3 | 1600/1600/200 |
| Example 37 | J-37 | A-1 | 100 | B-16 | 10.0 | D-1 | 4.0 | F-1 | 5.0 | E-1/E-2/E-3 | 1600/1600/200 |
| Example 103 | J-103 | A-1 | 100 | B-17 | 10.0 | D-1 | 4.0 | F-1 | 5.0 | E-1/E-2/E-3 | 1600/1600/200 |
| Example 104 | J-104 | A-1 | 100 | B-18 | 10.0 | D-1 | 4.0 | F-1 | 5.0 | E-1/E-2/E-3 | 1600/1600/200 |
| Example 105 | J-105 | A-1 | 100 | B-19 | 10.0 | D-1 | 4.0 | F-1 | 5.0 | E-1/E-2/E-3 | 1600/1600/200 |
| Example 106 | J-106 | A-1 | 100 | B-21 | 10.0 | D-1 | 4.0 | F-1 | 5.0 | E-1/E-2/E-3 | 1600/1600/200 |
| Example 107 | J-107 | A-1 | 100 | B-23 | 10.0 | D-1 | 4.0 | F-1 | 5.0 | E-1/E-2/E-3 | 1600/1600/200 |
| Example 38 | J-38 | A-1 | 100 | B-1/b-1 | 5.0/5.0 | D-1 | 4.0 | F-1 | 5.0 | E-1/E-2/E-3 | 1600/1600/200 |
| Example 39 | J-39 | A-1 | 100 | B-1/b-2 | 5.0/5.0 | D-1 | 4.0 | F-1 | 5.0 | E-1/E-2/E-3 | 1600/1600/200 |
| Example 40 | J-40 | A-1 | 100 | B-1/b-3 | 5.0/5.0 | D-1 | 4.0 | F-1 | 5.0 | E-1/E-2/E-3 | 1600/1600/200 |
| Example 41 | J-41 | A-1 | 100 | B-1/b-4 | 5.0/5.0 | D-1 | 4.0 | F-1 | 5.0 | E-1/E-2/E-3 | 1600/1600/200 |
| Example 42 | J-42 | A-1 | 100 | B-1/b-5 | 5.0/5.0 | D-1 | 4.0 | F-1 | 5.0 | E-1/E-2/E-3 | 1600/1600/200 |
| Example 43 | J-43 | A-1 | 100 | B-1/b-6 | 5.0/5.0 | D-1 | 4.0 | F-1 | 5.0 | E-1/E-2/E-3 | 1600/1600/200 |
| Example 44 | J-44 | A-1 | 100 | B-1/b-7 | 5.0/5.0 | D-1 | 4.0 | F-1 | 5.0 | E-1/E-2/E-3 | 1600/1600/200 |
| Example 45 | J-45 | A-1 | 100 | B-1 | 10.0 | D-1 | 4.0 | F-2 | 5.0 | E-1/E-2/E-3 | 1600/1600/200 |
| Example 46 | J-46 | A-1 | 100 | B-1 | 10.0 | D-1 | 4.0 | F-3 | 5.0 | E-1/E-2/E-3 | 1600/1600/200 |
| Example 47 | J-47 | A-1 | 100 | B-1 | 10.0 | D-1 | 4.0 | F-4 | 5.0 | E-1/E-2/E-3 | 1600/1600/200 |
| Example 108 | J-108 | A-1 | 100 | B-1 | 10.0 | D-1 | 4.0 | F-101 | 5.0 | E-1/E-2/E-3 | 1600/1600/200 |
| Example 109 | J-109 | A-1 | 100 | B-1 | 10.0 | D-1 | 4.0 | F-1 | 5.0 | E-1/E-2/E-4 | 2000/1200/30 |
| Example 110 | J-110 | A-1 | 100 | B-1 | 10.0 | D-1 | 4.0 | F-1 | 5.0 | E-1/E-2/E-5 | 2000/1200/30 |
| Comparative | CJ-1 | A-12 | 100 | B-1 | 10.0 | D-4 | 4.0 | F-1 | 5.0 | E-1/E-2/E-3 | 1600/1600/200 |
| Example 1 | | | | | | | | | | | |
| Comparative | CJ-2 | A-1 | 100 | b-8 | 10.0 | D-1 | 4.0 | F-1 | 5.0 | E-1/E-2/E-3 | 1600/1600/200 |
| Example 2 | | | | | | | | | | | |
| Comparative | CJ-3 | A-1 | 100 | b-9 | 10.0 | D-1 | 4.0 | F-1 | 5.0 | E-1/E-2/E-3 | 1600/1600/200 |
| Example 3 | | | | | | | | | | | |
| Comparative | CJ-4 | A-1 | 100 | b-10 | 10.0 | D-1 | 4.0 | F-1 | 5.0 | E-1/E-2/E-3 | 1600/1600/200 |
| Example 4 | | | | | | | | | | | |
| Comparative | CJ-5 | A-1 | 100 | b-11 | 10.0 | D-1 | 4.0 | F-1 | 5.0 | E-1/E-2/E-3 | 1600/1600/200 |
| Example 5 | | | | | | | | | | | |
| Comparative | CJ-21 | A-1 | 100 | b-12 | 10.0 | D-1 | 4.0 | F-1 | 5.0 | E-1/E-2/E-3 | 1600/1600/200 |
| Example 21 | | | | | | | | | | | |
| Comparative | CJ-22 | A-1 | 100 | b-13 | 10.0 | D-1 | 4.0 | F-1 | 5.0 | E-1/E-2/E-3 | 1600/1600/200 |
| Example 22 | | | | | | | | | | | |
| Comparative | CJ-23 | A-1 | 100 | b-14 | 10.0 | D-1 | 4.0 | F-1 | 5.0 | E-1/E-2/E-3 | 1600/1600/200 |
| Example 23 | | | | | | | | | | | |
| Comparative | CJ-24 | A-1 | 100 | b-15 | 10.0 | D-1 | 4.0 | F-1 | 5.0 | E-1/E-2/E-3 | 1600/1600/200 |
| Example 24 | | | | | | | | | | | |
| Comparative | CJ-25 | A-1 | 100 | b-3 | 10.0 | D-1 | 4.0 | F-1 | 5.0 | E-1/E-2/E-3 | 1600/1600/200 |
| Example 25 | | | | | | | | | | | |

### <Formation of Resist Pattern Using Positive Radiation-Sensitive Composition for ArF Immersion Exposure>

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 100 nm. The positive radiation-sensitive composition for ArF immersion exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 160 nm. Next, this resist film was exposed through a mask pattern of contact holes with an 80 nm hole and a 150 nm pitch using an ArF excimer laser immersion exposure apparatus (“TWINSCAN XT-1900i” manufactured by ASML) at NA=1.35 under an optical condition of Annular (σ=0.8/0.6). After the exposure, PEB (post exposure bake) was performed at 100° C. for 60 seconds. Thereafter, the resist film was developed with an alkali with use of a 2.38% by mass aqueous TMAH solution as an alkaline developer, followed by washing with water and further drying to form a positive resist pattern (pattern of contact holes with an 80 nm hole and a 150 nm pitch).

### <Evaluation>

The resist pattern formed using the positive radiation-sensitive composition for ArF immersion exposure was evaluated on sensitivity, CDU performance, pattern circularity, and the number of development defects according to the following methods. The results are shown in Table 5 below. A scanning electron microscope (“CG-5000” manufactured by Hitachi High-Tech Corporation) was used for measuring the length of the resist pattern.

### [Sensitivity]

An exposure dose at which contact holes with an 80 nm hole and a 150 nm pitch were formed in the resist pattern formation using the positive radiation-sensitive compositions for ArF immersion exposure was defined as an optimum exposure dose, and this optimum exposure dose was defined as sensitivity (mJ/cm²). In the case of being 30 mJ/cm² or less, the sensitivity was evaluated as “good”, and in the case of being more than 30 mJ/cm², the sensitivity was evaluated as “poor”.

### [CDU Performance]

Contact holes with an 80 nm hole and a 150 nm pitch were formed by irradiation with an optimum exposure dose determined in the evaluation of sensitivity. The formed resist pattern was observed from above the pattern using the scanning electron microscope. The variation of the contact holes was measured at 500 points in total, and the 3 sigma value was determined from the distribution of the measurement values, and defined as CDU (nm). The smaller the value of the CDU is, the smaller the roughness of the contact holes is, which is better. When the value was less than 3.0 nm, the CDU performance was evaluated to be “good”, and when the value was 3.0 nm or more, the CDU performance was evaluated to be “poor”.

### [Pattern Circularity]

The contact holes with an 80 nm hole and a 150 nm pitch formed by irradiation with the optimum exposure dose determined in the evaluation of sensitivity were observed using the scanning electron microscope, and the size in the longitudinal direction and the size in the lateral direction were measured. When the ratio of the size in the longitudinal direction to the size in the lateral direction (aspect ratio) was 0.95 or more and less than 1.05, the pattern circularity was evaluated as “A” (extremely good), when the ratio was 0.90 or more and less than 0.95, or 1.05 or more and less than 1.10, the pattern circularity was evaluated as “B” (good), and when the ratio was less than 0.90, or 1.10 or more, the pattern circularity was evaluated as “C” (poor).

### [Number of Development Defects]

The resist film was exposed at the optimum exposure dose to form a contact hole pattern having 80 nm holes and 150 nm pitches, thereby obtaining a wafer for defect inspection. The number of defects on this wafer for defect inspection was measured with use of a defect inspection device (“KLA 2810” of KLA-Tencor Corporation). The defects having a diameter of 50 nm or less were determined to be derived from the resist film, and the number of the defects was calculated. When the number of the defects determined to be derived from the resist film was 50 or less, the number of defects after development was evaluated as “good”, and when more than 50, evaluated as “poor”.

### [EL (Exposure Latitude)]

In the range of the exposure dose including the optimum exposure dose, resist patterns were formed by changing the exposure dose every 1 mJ/cm², and the hole diameter of each resist pattern was measured using the scanning electron microscope. From the obtained relationship between the hole diameter and the exposure dose, the exposure dose E(88) at which the hole diameter was 88 nm and the exposure dose E(72) at which the hole diameter was 72 nm were determined, and the exposure latitude (%) was calculated from the equation: exposure latitude (EL)=(E(72)−E(88))×100/(optimum exposure dose). The larger the value of the exposure latitude, the smaller the fluctuation in dimension among the patterns obtained when the exposure dose fluctuates, and the higher the yield at the time of manufacturing a device. When being 10% or more, the EL was evaluated as “good”, and when being less than 10%, the EL was evaluated as “poor”.

TABLE 5

| | | | | | Number of | |
|---|---|---|---|---|---|---|
| | Radiation-sensitive | Sensitivity | CDU | Pattern | development | EL |
| | composition | (mJ/cm²) | (nm) | circularity | defects | (%) |
| Example 1 | J-1 | 24 | 2.2 | A | 12 | 15.7 |
| Example 2 | J-2 | 20 | 2.1 | A | 23 | 17.5 |
| Example 3 | J-3 | 26 | 2.3 | A | 21 | 11.2 |
| Example 4 | J-4 | 26 | 2.0 | A | 11 | 11.7 |
| Example 5 | J-5 | 27 | 1.9 | A | 19 | 14.0 |
| Example 6 | J-6 | 26 | 2.1 | A | 23 | 12.1 |
| Example 7 | J-7 | 23 | 2.4 | A | 43 | 12.5 |
| Example 8 | J-8 | 25 | 1.8 | A | 12 | 17.1 |
| Example 9 | J-9 | 20 | 2.5 | A | 24 | 11.6 |
| Example 10 | J-10 | 23 | 2.4 | A | 31 | 11.7 |
| Example 11 | J-11 | 26 | 1.8 | A | 22 | 15.6 |
| Example 101 | J-101 | 22 | 1.7 | A | 5 | 14.7 |
| Example 102 | J-102 | 21 | 1.7 | A | 12 | 15.2 |
| Example 12 | J-12 | 25 | 2.1 | A | 13 | 12.1 |
| Example 13 | J-13 | 24 | 1.8 | A | 14 | 17.1 |
| Example 14 | J-14 | 21 | 2.5 | A | 21 | 12.1 |
| Example 15 | J-15 | 22 | 2.1 | A | 22 | 11.8 |
| Example 16 | J-16 | 20 | 1.9 | A | 19 | 16.5 |
| Example 17 | J-17 | 20 | 1.8 | A | 15 | 14.4 |
| Example 18 | J-18 | 21 | 2.0 | A | 33 | 12.2 |
| Example 19 | J-19 | 26 | 2.1 | A | 13 | 12.0 |
| Example 20 | J-20 | 25 | 2.2 | A | 15 | 11.6 |
| Example 21 | J-21 | 26 | 1.7 | A | 13 | 15.9 |
| Example 22 | J-22 | 23 | 2.4 | A | 26 | 11.7 |
| Example 23 | J-23 | 25 | 2.1 | A | 10 | 16.4 |
| Example 24 | J-24 | 25 | 2.6 | A | 22 | 14.4 |
| Example 25 | J-25 | 27 | 2.6 | A | 21 | 14.9 |
| Example 26 | J-26 | 20 | 2.1 | A | 25 | 15.2 |
| Example 27 | J-27 | 20 | 2.3 | A | 13 | 14.7 |
| Example 28 | J-28 | 27 | 2.5 | A | 18 | 16.6 |
| Example 29 | J-29 | 23 | 2.0 | A | 20 | 17.2 |
| Example 30 | J-30 | 25 | 2.4 | A | 21 | 16.7 |
| Example 31 | J-31 | 22 | 1.8 | A | 23 | 17.8 |
| Example 32 | J-32 | 20 | 1.7 | A | 32 | 15.5 |
| Example 33 | J-33 | 25 | 1.9 | A | 22 | 17.5 |
| Example 34 | J-34 | 26 | 1.6 | A | 21 | 15.1 |
| Example 35 | J-35 | 21 | 1.8 | A | 28 | 18.0 |
| Example 36 | J-36 | 25 | 1.8 | A | 29 | 14.6 |
| Example 37 | J-37 | 26 | 1.7 | A | 31 | 15.0 |
| Example 103 | J-103 | 23 | 2.2 | A | 21 | 14.8 |
| Example 104 | J-104 | 24 | 2.1 | A | 23 | 15.8 |
| Example 105 | J-105 | 22 | 1.6 | A | 20 | 17.6 |
| Example 106 | J-106 | 26 | 1.7 | A | 13 | 16.5 |
| Example 107 | J-107 | 22 | 1.6 | A | 9 | 17.8 |
| Example 38 | J-38 | 20 | 2.1 | A | 12 | 14.8 |
| Example 39 | J-39 | 21 | 2.2 | A | 13 | 17.8 |
| Example 40 | J-40 | 25 | 2.2 | A | 15 | 14.7 |
| Example 41 | J-41 | 27 | 2.3 | A | 13 | 16.5 |
| Example 42 | J-42 | 25 | 2.1 | A | 12 | 16.7 |
| Example 43 | J-43 | 23 | 2.2 | A | 18 | 14.3 |
| Example 44 | J-44 | 27 | 2.1 | A | 17 | 14.7 |
| Example 45 | J-45 | 24 | 2.3 | A | 21 | 17.4 |
| Example 46 | J-46 | 23 | 2.2 | A | 24 | 17.6 |
| Example 47 | J-47 | 24 | 2.2 | A | 25 | 15.7 |
| Example 108 | J-108 | 24 | 2.2 | A | 40 | 15.5 |
| Example 109 | J-109 | 23 | 2.2 | A | 12 | 15.4 |
| Example 110 | J-110 | 21 | 2.3 | A | 25 | 15.7 |
| Comparative | CJ-1 | 41 | 3.7 | C | 140 | 4.5 |
| Example 1 | | | | | | |
| Comparative | CJ-2 | 38 | 3.5 | C | 231 | 5.2 |
| Example 2 | | | | | | |
| Comparative | CJ-3 | 43 | 3.4 | C | 320 | 4.4 |
| Example 3 | | | | | | |
| Comparative | CJ-4 | 39 | 3.6 | C | 72 | 4.2 |
| Example 4 | | | | | | |
| Comparative | CJ-5 | 37 | 3.5 | B | 67 | 8.9 |
| Example 5 | | | | | | |
| Comparative | CJ-21 | 37 | 3.5 | B | 98 | 9.3 |
| Example | | | | | | |
| 21 | | | | | | |
| Comparative | CJ-22 | 39 | 4.0 | B | 78 | 7.8 |
| Example 22 | | | | | | |
| Comparative | CJ-23 | 41 | 3.3 | C | 72 | 4.5 |
| Example 23 | | | | | | |
| Comparative | CJ-24 | 40 | 4.1 | C | 67 | 6.4 |
| Example 24 | | | | | | |
| Comparative | CJ-25 | 43 | 3.5 | C | 421 | 4.4 |
| Example 25 | | | | | | |

As is apparent from the results in Table 5, the radiation-sensitive compositions of Examples were good in sensitivity, CDU performance, pattern circularity, development defect suppression performance, and EL when used for ArF exposure, whereas the radiation-sensitive compositions of Comparative Examples were inferior in the characteristics to those of Examples. Therefore, when the radiation-sensitive compositions of Examples are used for ArF exposure, resist patterns having good CDU performance, pattern circularity, development defect suppression performance, and EL can be formed with high sensitivity.

### [Preparation of Negative Radiation-Sensitive Composition for ArF Immersion Exposure]

### Example 48

After mixing 100 parts by mass of (A-1) as the polymer [A], 6.0 parts by mass of (B-1) as the onium salt compound [B], 3.0 parts by mass of (D-2) as the acid diffusion controlling agent [D], 3.0 parts by mass (solid content) of (F-3) as the high fluorine-content polymer [F], and 3,230 parts by mass of a mixed solvent of (E-1)/(E-2)/(E-3) as the solvent [E], the mixture was filtered through a membrane filter having a pore diameter of 0.2 μm to prepare a radiation-sensitive composition (J-48).

### Examples 49 to 61 and Comparative Examples 6 to 10

Radiation-sensitive compositions (J-49) to (J-61) and (CJ-6) to (CJ-10) were prepared in the same manner as in Example 48 except that the components of the types and contents shown in Table 6 below were used.

TABLE 6

| | | | | | | Acid diffusion | | | | | |
|---|---|---|---|---|---|---|---|---|---|---|---|
| | | | | Onium salt | | controlling agent | | High fluorine- | | | |
| | | Polymer [A] | | compound [B] | | [D] | | content polymer [F] | | Solvent [E] | |
| | Radiation- | | Content | | Content | | Content | | Content | | Content |
| | sensitive | | (parts by | | (parts by | | (parts by | | (parts by | | (parts by |
| | composition | Type | mass) | Type | mass) | Type | mass) | Type | mass) | Type | mass) |
| Example 48 | J-48 | A-1 | 100 | B-1 | 6.0 | D-2 | 3.0 | F-3 | 3.0 | E-1/E-2/E-3 | 2240/960/30 |
| Example 49 | J-49 | A-6 | 100 | B-1 | 6.0 | D-2 | 3.0 | F-3 | 3.0 | E-1/E-2/E-3 | 2240/960/30 |
| Example 50 | J-50 | A-7 | 100 | B-1 | 6.0 | D-2 | 3.0 | F-3 | 3.0 | E-1/E-2/E-3 | 2240/960/30 |
| Example 51 | J-51 | A-8 | 100 | B-1 | 6.0 | D-2 | 3.0 | F-3 | 3.0 | E-1/E-2/E-3 | 2240/960/30 |
| Example 52 | J-52 | A-1 | 100 | B-1 | 6.0 | D-5 | 3.0 | F-3 | 3.0 | E-1/E-2/E-3 | 2240/960/30 |
| Example 53 | J-53 | A-1 | 100 | B-1 | 6.0 | D-7 | 3.0 | F-3 | 3.0 | E-1/E-2/E-3 | 2240/960/30 |
| Example 54 | J-54 | A-1 | 100 | B-4 | 6.0 | D-2 | 3.0 | F-3 | 3.0 | E-1/E-2/E-3 | 2240/960/30 |
| Example 55 | J-55 | A-1 | 100 | B-5 | 6.0 | D-2 | 3.0 | F-3 | 3.0 | E-1/E-2/E-3 | 2240/960/30 |
| Example 56 | J-56 | A-1 | 100 | B-8 | 6.0 | D-2 | 3.0 | F-3 | 3.0 | E-1/E-2/E-3 | 2240/960/30 |
| Example 57 | J-57 | A-1 | 100 | B-9 | 6.0 | D-2 | 3.0 | F-3 | 3.0 | E-1/E-2/E-3 | 2240/960/30 |
| Example 58 | J-58 | A-1 | 100 | B-12 | 6.0 | D-2 | 3.0 | F-3 | 3.0 | E-1/E-2/E-3 | 2240/960/30 |
| Example 59 | J-59 | A-1 | 100 | B-8/b-4 | 3.0/3.0 | D-2 | 3.0 | F-3 | 3.0 | E-1/E-2/E-3 | 2240/960/30 |
| Example 60 | J-60 | A-1 | 100 | B-13/b-6 | 3.0/3.0 | D-2 | 3.0 | F-3 | 3.0 | E-1/E-2/E-3 | 2240/960/30 |
| Example 61 | J-61 | A-1 | 100 | B-14/b-7 | 3.0/3.0 | D-2 | 3.0 | F-3 | 3.0 | E-1/E-2/E-3 | 2240/960/30 |
| Comparative | CJ-6 | A-12 | 100 | B-1 | 6.0 | D-2 | 3.0 | F-3 | 3.0 | E-1/E-2/E-3 | 2240/960/30 |
| Example 6 | | | | | | | | | | | |
| Comparative | CJ-7 | A-1 | 100 | b-8 | 6.0 | D-2 | 3.0 | F-3 | 3.0 | E-1/E-2/E-3 | 2240/960/30 |
| Example 7 | | | | | | | | | | | |
| Comparative | CJ-8 | A-1 | 100 | b-9 | 6.0 | D-2 | 3.0 | F-3 | 3.0 | E-1/E-2/E-3 | 2240/960/30 |
| Example 8 | | | | | | | | | | | |
| Comparative | CJ-9 | A-1 | 100 | b-10 | 6.0 | D-2 | 3.0 | F-3 | 3.0 | E-1/E-2/E-3 | 2240/960/30 |
| Example 9 | | | | | | | | | | | |
| Comparative | CJ-10 | A-1 | 100 | b-11 | 6.0 | D-2 | 3.0 | F-3 | 3.0 | E-1/E-2/E-3 | 2240/960/30 |
| Example 10 | | | | | | | | | | | |

### <Formation of Resist Pattern Using Negative Radiation-Sensitive Composition for ArF Immersion Exposure>

Onto the surface of a 12-inch silicon wafer, an underlayer antireflection film forming composition (“ARC66” manufactured by Brewer Science Incorporated) was applied with use of a spin coater (“CLEAN TRACK ACT12” manufactured by Tokyo Electron Limited). The wafer was then heated at 205° C. for 60 seconds to form an underlayer antireflection film having an average thickness of 100 nm. The negative radiation-sensitive composition for ArF immersion exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 90 nm. Next, this resist film was exposed through a mask pattern having a 40 nm hole and a 105 nm pitch using an ArF excimer laser immersion exposure apparatus (“TWINSCAN XT-1900i” manufactured by ASML) with NA of 1.35 under an optical condition of Dipole (σ=0.9/0.7). After the exposure, PEB (post exposure bake) was performed at 100° C. for 60 seconds. Thereafter, the resist film was developed with an organic solvent using n-butyl acetate as an organic solvent developer, and dried to form a negative resist pattern (40 nm hole, 105 nm pitch).

### <Evaluation>

The resist pattern formed using the negative radiation-sensitive composition for ArF immersion exposure was evaluated on sensitivity, CDU performance, and depth of focus in accordance with the following methods. The results are shown in the following Table 7. A scanning electron microscope (“CG-5000” manufactured by Hitachi High-Tech Corporation) was used for measuring the length of the resist pattern.

### [Sensitivity]

An exposure dose at which a 40 nm hole and 105 nm pitch resist pattern was formed in formation of a resist pattern using the negative radiation-sensitive composition for ArF immersion exposure was defined as the optimum exposure dose, and this optimum exposure dose was defined as sensitivity (mJ/cm²). The sensitivity was evaluated to be “good” in the case of being 25 mJ/cm² or less, and “poor” in the case of exceeding 25 mJ/cm².

### [CDU Performance]

A resist pattern with 40 nm holes and 105 nm pitches was measured using the scanning electron microscope, and measurement was performed at any 1,800 points in total from above the pattern. The dimensional variation (3o) was determined and taken as the CDU performance (nm). A smaller value of CDU indicates smaller variation in the hole diameter in the long period and better performance. When the value was 4.0 nm or less, the CDU performance was evaluated as “good”, and when the value exceeded 4.0 nm, the CDU performance was evaluated as “poor”.

### [Depth of Focus]

In a resist pattern to be resolved with the optimum exposure dose determined in the above-described evaluation of sensitivity, the dimension when the focus was changed in the depth direction was observed, and the latitude in the depth direction in which the pattern dimension fell within 90% to 110% of the reference without any bridge or residue was measured. The measured value was taken as the depth of focus (nm). The larger the measured value, the better the depth of focus. When the measured value is 70 nm or more, the depth of focus can be evaluated as “good”, and when the measured value is less than 70 nm, the depth of focus can be evaluated as “poor”

TABLE 7

| | Radiation- | Sensi- | | |
|---|---|---|---|---|
| | sensitive | tivity | CDU | Depth of |
| | composition | (mJ/cm²) | (nm) | focus |
| Example 48 | J-48 | 15 | 3.3 | 100 |
| Example 49 | J-49 | 18 | 3.6 | 120 |
| Example 50 | J-50 | 18 | 2.8 | 110 |
| Example 51 | J-51 | 19 | 2.8 | 100 |
| Example 52 | J-52 | 20 | 3.5 | 130 |
| Example 53 | J-53 | 19 | 2.8 | 120 |
| Example 54 | J-54 | 21 | 3.5 | 110 |
| Example 55 | J-55 | 22 | 3.6 | 100 |
| Example 56 | J-56 | 16 | 3.1 | 100 |
| Example 57 | J-57 | 19 | 2.9 | 100 |
| Example 58 | J-58 | 20 | 2.8 | 120 |
| Example 59 | J-59 | 21 | 2.7 | 110 |
| Example 60 | J-60 | 15 | 3.1 | 120 |
| Example 61 | J-61 | 22 | 3.0 | 110 |
| Comparative | CJ-6 | 33 | 4.9 | 20 |
| Example 6 | | | | |
| Comparative | CJ-7 | 34 | 4.7 | 30 |
| Example 7 | | | | |
| Comparative | CJ-8 | 35 | 4.5 | 20 |
| Example 8 | | | | |
| Comparative | CJ-9 | 37 | 4.5 | 50 |
| Example 9 | | | | |
| Comparative | CJ-10 | 36 | 4.6 | 50 |
| Example 10 | | | | |

As is apparent from the results in Table 7, the radiation-sensitive compositions of Examples were good in sensitivity, CDU performance, and depth of focus when used for ArF exposure, whereas the radiation-sensitive compositions of Comparative Examples were poorer in the characteristics than those of Examples. Therefore, when the radiation-sensitive compositions of Examples are used for ArF exposure, a resist pattern having good CDU performance and depth of focus with optimum sensitivity can be formed.

### [Preparation of Positive Radiation-Sensitive Composition for Extreme Ultraviolet (EUV) Exposure]

### Example 62

After mixing 100 parts by mass of (A-13) as the polymer [A], 20.0 parts by mass of (B-1) as the onium salt compound [B], 10.0 parts by mass of (D-1) as the acid diffusion controlling agent [D], 3.0 parts by mass (solid content) of (F-5) as the high fluorine-content polymer [F], and 6,100 parts by mass of a mixed solvent of (E-1)/(E-2)/(E-4) as the solvent [E], the mixture was filtered through a membrane filter having a pore diameter of 0.2 μm to prepare a radiation-sensitive composition (J-62).

### Examples 63 to 72 and 111 to 117 and Comparative Examples 11 to 15, 26, and 27

Radiation-sensitive compositions (J-63) to (J-72), (J-111) to (J-117), (CJ-11) to (CJ-15), (CJ-26), and (CJ-27) were prepared in the same manner as in Example 62 except that the respective components of the types and contents shown in Table 8 below were used.

TABLE 8

| | | | | | | Acid diffusion | | High fluorine- | | | |
|---|---|---|---|---|---|---|---|---|---|---|---|
| | | | | Onium salt compound | | controlling agent | | content polymer | | | |
| | | Polymer [A] | | [B] | | [D] | | [F] | | Solvent [E] | |
| | Radiation- | | Content | | Content | | Content | | Content | | Content |
| | sensitive | | (parts by | | (parts by | | (parts by | | (parts by | | (parts by |
| | composition | Type | mass) | Type | mass) | Type | mass) | Type | mass) | Type | mass) |
| Example 62 | J-62 | A-13 | 100 | B-1 | 20.0 | D-1 | 10.0 | F-5 | 3.0 | E-1/E-2/E-4 | 2000/4000/100 |
| Example 63 | J-63 | A-13 | 100 | B-2 | 20.0 | D-1 | 10.0 | F-5 | 3.0 | E-1/E-2/E-4 | 2000/4000/100 |
| Example 64 | J-64 | A-13 | 100 | B-4 | 20.0 | D-1 | 10.0 | F-5 | 3.0 | E-1/E-2/E-4 | 2000/4000/100 |
| Example 65 | J-65 | A-13 | 100 | B-10 | 20.0 | D-1 | 10.0 | F-5 | 3.0 | E-1/E-2/E-4 | 2000/4000/100 |
| Example 66 | J-66 | A-13 | 100 | B-11 | 20.0 | D-1 | 10.0 | F-5 | 3.0 | E-1/E-2/E-4 | 2000/4000/100 |
| Example 67 | J-67 | A-13 | 100 | B-14 | 20.0 | D-1 | 10.0 | F-5 | 3.0 | E-1/E-2/E-4 | 2000/4000/100 |
| Example 111 | J-111 | A-13 | 100 | B-18 | 20.0 | D-1 | 10.0 | F-5 | 3.0 | E-1/E-2/E-4 | 2000/4000/100 |
| Example 112 | J-112 | A-13 | 100 | B-20 | 20.0 | D-1 | 10.0 | F-5 | 3.0 | E-1/E-2/E-4 | 2000/4000/100 |
| Example 113 | J-113 | A-13 | 100 | B-22 | 20.0 | D-1 | 10.0 | F-5 | 3.0 | E-1/E-2/E-4 | 2000/4000/100 |
| Example 114 | J-114 | A-13 | 100 | B-20 | 40.0 | D-1 | 20.0 | F-5 | 3.0 | E-1/E-2/E-4 | 2000/4000/100 |
| Example 68 | J-68 | A-13 | 100 | B-20/b-2 | 40.0/10.0 | D-1 | 20.0 | F-5 | 3.0 | E-1/E-2/E-4 | 2000/4000/100 |
| Example 69 | J-69 | A-13 | 100 | B-20/b-5 | 40.0/10.0 | D-1 | 20.0 | F-5 | 3.0 | E-1/E-2/E-4 | 2000/4000/100 |
| Example 70 | J-70 | A-14 | 100 | B-20 | 20.0 | D-1 | 10.0 | F-5 | 3.0 | E-1/E-2/E-4 | 2000/4000/100 |
| Example 71 | J-71 | A-15 | 100 | B-20 | 20.0 | D-1 | 10.0 | F-5 | 3.0 | E-1/E-2/E-4 | 2000/4000/100 |
| Example 72 | J-72 | A-16 | 100 | B-20 | 20.0 | D-1 | 10.0 | F-5 | 3.0 | E-1/E-2/E-4 | 2000/4000/100 |
| Example 115 | J-115 | A-103 | 100 | B-20 | 20.0 | D-1 | 10.0 | F-5 | 3.0 | E-1/E-2/E-4 | 2000/4000/100 |
| Example 116 | J-116 | A-13 | 100 | B-20 | 20.0 | D-1 | 10.0 | F-5 | 3.0 | E-1/E-5/E-3 | 2000/4000/100 |
| Example 117 | J-117 | A-13 | 100 | B-20 | 20.0 | D-1 | 10.0 | F-5 | 3.0 | E-1/E-5/E-3 | 2000/4000/100 |
| Comparative | CJ-11 | A-17 | 100 | B-1 | 20.0 | D-1 | 10.0 | F-5 | 3.0 | E-1/E-2/E-4 | 2000/4000/100 |
| Example 11 | | | | | | | | | | | |
| Comparative | CJ-12 | A-13 | 100 | b-8 | 20. | D-1 | 10.0 | F-5 | 3.0 | E-1/E-2/E-4 | 2000/4000/100 |
| Example 12 | | | | | | | | | | | |
| Comparative | CJ-13 | A-13 | 100 | b-3 | 20.0 | D-1 | 10.0 | F-5 | 3.0 | E-1/E-2/E-4 | 2000/4000/100 |
| Example 13 | | | | | | | | | | | |
| Comparative | CJ-14 | A-13 | 100 | b-10 | 20.0 | D-1 | 10.0 | F-5 | 3.0 | E-1/E-2/E-4 | 2000/4000/100 |
| Example 14 | | | | | | | | | | | |
| Comparative | CJ-15 | A-13 | 100 | b-11 | 20.0 | D-1 | 10.0 | F-5 | 3.0 | E-1/E-2/E-4 | 2000/4000/100 |
| Example 15 | | | | | | | | | | | |
| Comparative | CJ-26 | A-13 | 100 | b-12 | 20.0 | D-1 | 10.0 | F-5 | 3.0 | E-1/E-2/E-4 | 2000/4000/100 |
| Example 26 | | | | | | | | | | | |
| Comparative | CJ-27 | A-13 | 100 | b-13 | 20.0 | D-1 | 10.0 | F-5 | 3.0 | E-1/E-2/E-4 | 2000/4000/100 |
| Example 27 | | | | | | | | | | | |

### <Formation of Resist Pattern Using Positive Radiation-Sensitive Composition for EUN Exposure>

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 105 nm. The positive radiation-sensitive composition for EUV exposure prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed by an EUV exposure apparatus (“NXE3300”, manufactured by ASML) with NA of 0.33 under a lighting condition of Conventional s=0.89 and with a mask of imecDEFECT32FFR02. After exposing, PEB was performed at 120° C. for 60 seconds. Thereafter, the resist film was developed with an alkali with use of a 2.38% by mass aqueous TMAH solution as an alkaline developer, followed by washing with water and further drying to form a positive resist pattern (15 nm line-and-space pattern).

### <Evaluation>

The resist pattern formed using the positive radiation-sensitive composition for EUV exposure was evaluated on sensitivity, LWR performance, pattern rectangularity in accordance with the following methods. The results are shown in the following Table 9. A scanning electron microscope (“CG-5000” manufactured by Hitachi High-Tech Corporation) was used for measuring the length of the resist pattern.

### [Sensitivity]

An exposure dose at which a 15 nm line-and-space pattern was formed in the aforementioned resist pattern formation using the positive radiation-sensitive composition for EUV exposure was defined as an optimum exposure dose, and this optimum exposure dose was defined as sensitivity (mJ/cm²). The sensitivity was evaluated to be “good” in a case of being 35 mJ/cm² or less, and “poor” in a case of exceeding 35 mJ/cm².

### [LWR Performance]

A resist pattern was formed by adjusting a mask size so as to form a 15 nm line-and-space pattern by irradiation with the optimum exposure dose obtained in the evaluation of the sensitivity. The formed resist pattern was observed from above the pattern with use of the scanning electron microscope. The variation in the line width was measured at a total of 500 points. The 3 sigma value was obtained from the distribution of the measurement values, and defined as LWR (nm). The smaller the value of the LWR is, the smaller the wobble of the line is, which is better. The LWR performance was evaluated to be “good” in a case of being 2.5 nm or less, and “poor” in a case of exceeding 2.5 nm.

### [Pattern Rectangularity]

The 15 nm line-and-space resist pattern formed by irradiation with the optimum exposure dose determined in the evaluation of the sensitivity was observed with use of the scanning electron microscope, and the sectional shape of the line-and-space pattern was evaluated. The rectangularity of the resist pattern was evaluated as “A” (extremely good) when the ratio of the length of the lower side to the length of the upper side in the sectional shape was 1.00 or more and 1.05 or less, “B” (good) when the ratio was more than 1.05 and 1.10 or less, and “C” (poor) when the ratio was more than 1.10.

### [EL (Exposure Latitude)]

In the range of the exposure dose including the optimum exposure dose, resist patterns were formed by changing the exposure dose every 1 mJ/cm², and the line width of each resist pattern was measured using the scanning electron microscope. From the obtained relationship between the line width and the exposure dose, the exposure dose E(16.5) at which the line width was 16.5 nm and the exposure dose E(13.5) at which the line width was 13.5 nm were determined, and the exposure latitude (%) was calculated from the equation: exposure latitude (EL)=(E(13.5)−E(16.5))×100/(optimum exposure dose). The larger the value of the exposure latitude, the smaller the fluctuation in dimension among the patterns obtained when the exposure dose fluctuates, and the higher the yield at the time of manufacturing a device. When being 7% or more, the EL was evaluated as “good”, and when being less than 7%, the EL was evaluated as “poor”.

TABLE 9

| | Radiation- | Sensi- | | Pattern | |
|---|---|---|---|---|---|
| | sensitive | tivity | LWR | rectan- | EL |
| | composition | (mJ/cm²) | (nm) | gularity | (%) |
| Example 62 | J-62 | 29 | 2.1 | A | 8.0 |
| Example 63 | J-63 | 26 | 2.2 | A | 8.4 |
| Example 64 | J-64 | 25 | 2.2 | A | 8.5 |
| Example 65 | J-65 | 31 | 1.9 | A | 11.2 |
| Example 66 | J-66 | 26 | 1.5 | A | 11.8 |
| Example 67 | J-67 | 26 | 1.7 | A | 12.5 |
| Example 111 | J-111 | 27 | 2.2 | A | 9.0 |
| Example 112 | J-112 | 27 | 1.7 | A | 12.1 |
| Example 113 | J-113 | 28 | 1.8 | A | 13.7 |
| Example 114 | J-114 | 25 | 1.7 | A | 11.4 |
| Example 68 | J-68 | 29 | 2.0 | A | 8.3 |
| Example 69 | J-69 | 26 | 2.0 | A | 8.5 |
| Example 70 | J-70 | 27 | 1.8 | A | 11.2 |
| Example 71 | J-71 | 29 | 1.9 | A | 13.8 |
| Example 72 | J-72 | 30 | 1.7 | A | 13.3 |
| Example 115 | J-115 | 27 | 1.8 | A | 12.4 |
| Example 116 | J-116 | 27 | 1.8 | A | 12.9 |
| Example 117 | J-117 | 27 | 1.9 | A | 12.3 |
| Comparative | CJ-11 | 45 | 3.6 | C | 3.4 |
| Example 11 | | | | | |
| Comparative | CJ-12 | 40 | 3.5 | C | 3.8 |
| Example 12 | | | | | |
| Comparative | CJ-13 | 42 | 3.5 | C | 4.3 |
| Example 13 | | | | | |
| Comparative | CJ-14 | 46 | 3.4 | C | 5.5 |
| Example 14 | | | | | |
| Comparative | CJ-15 | 43 | 3.4 | C | 5.4 |
| Example 15 | | | | | |
| Comparative | CJ-26 | 46 | 3.8 | B | 5.9 |
| Example 26 | | | | | |
| Comparative | CJ-27 | 44 | 3.9 | C | 6.0 |
| Example 27 | | | | | |

As is apparent from the results in Table 9, the radiation-sensitive compositions of Examples were good in sensitivity, LWR performance, pattern rectangularity, and EL when used for EUV exposure, whereas the radiation-sensitive compositions of Comparative Examples were inferior in the characteristics to those of Examples.

[Preparation of Negative Radiation-Sensitive Composition for EUV Exposure, and Formation and Evaluation of Resist Pattern Using this Composition]

### Example 73

After mixing 100 parts by mass of (A-16) as the polymer [A], 30.0 parts by mass of (B-12) as the onium salt compound [B], 10.0 parts by mass of (D-4) as the acid diffusion controlling agent [D], 5.0 parts by mass (solid content) of (F-5) as the high fluorine-content polymer [F], and 6,110 parts by mass of a mixed solvent of (E-1)/(E-4) (4280/1830 parts by mass) as the solvent [E], the mixture was filtered through a membrane filter having a pore diameter of 0.2 μm to prepare a radiation-sensitive composition (J-73).

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 105 nm. The negative radiation-sensitive composition for EUV exposure (J-73) prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed by an EUV exposure apparatus (“NXE3300”, manufactured by ASML) with NA of 0.33 under a lighting condition of Conventional s=0.89 and with a mask of imecDEFECT32FFR15. After exposing, PEB was performed at 120° C. for 60 seconds. Thereafter, the resist film was developed with an organic solvent using n-butyl acetate as an organic solvent developer, and dried to form a negative resist pattern (contact hole pattern with hole of 20 nm and pitch of nm).

The resist pattern formed using the negative radiation-sensitive composition for EUV exposure was evaluated in the same manner as the resist pattern formed using the negative radiation-sensitive composition for ArF exposure. As a result, the radiation-sensitive composition of Example 73 had good sensitivity, CDU performance, and depth of focus even when a negative resist pattern was formed by EUV exposure.

According to the radiation-sensitive composition and the method for forming a pattern described above, a resist pattern having good sensitivity to exposure light and excellent CDU performance, pattern circularity, development defect suppression performance, depth of focus, LWR performance, pattern rectangularity, and exposure latitude can be formed. Therefore, the composition and method can be suitably used for a machining process and the like of a semiconductor device that is expected to be further miniaturized in the future.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising:
an onium salt compound represented by formula (1);
a polymer comprising a structural unit (I) represented by formula (2); and
a solvent, 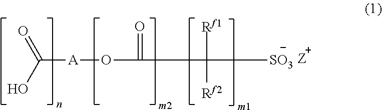
wherein in the formula (1),
A is a (1+n)-valent organic group having 3 to 40 carbon atoms comprising a cyclic structure, when the group represented by A comprises a linear alkanediyl group, a carbon number of the linear alkanediyl group is 1 or 2, the group represented by A does not contain a cyclohexylcarbonyloxy structure,
R^{f1} and R^{f2} are each independently a hydrogen atom, a monovalent organic group having 1 to 20 carbon atoms, a fluorine atom, or a monovalent fluorinated hydrocarbon group, when there are a plurality of R^{f1}s and R^{f2}s, the plurality of R^{f1}s and R^{f2}s are the same or different from each other, at least one of R^{f1} and R^{f2} is a fluorine atom or a monovalent fluorinated hydrocarbon group,
ml is an integer of 1 to 4,
m2 is 0 or 1,
n is an integer of 1 to 3,
Z⁺ is a monovalent radiation-sensitive onium cation, and the number of fluorine atoms in Z⁺ is 8 or less, 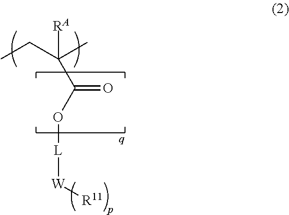
wherein in the formula (2),
R^{A} is a hydrogen atom, a fluorine atom, a methyl group, or a trifluoromethyl group,
L is a single bond or a divalent linking group,
W is a monocyclic lactone structure, a monocyclic carbonate structure, a monocyclic sultone structure, a monocyclic sulfone structure, or a monocyclic ether structure,
R¹¹ is a monovalent organic group having 1 to 10 carbon atoms, a cyano group, a nitro group, a hydroxy group, or an amino group, when there are a plurality of R¹¹s, the plurality of R¹¹s are each the same or different from each other,
p is an integer of 0 to 3, and
q is 0 or 1.

2. The radiation-sensitive composition according to claim 1, wherein the cyclic structure included in the group represented by A is an alicyclic hydrocarbon structure, an aromatic hydrocarbon structure, a lactone structure, a sultone structure, a cyclic acetal structure, a cyclic carbonate structure, a cyclic ether structure, or a combination thereof.

3. The radiation-sensitive composition according to claim 1, wherein in the formula (1), n is 1 or 2.

4. The radiation-sensitive composition according to claim 1, wherein a content of the onium salt compound in the radiation-sensitive composition is 1 part by mass or more and 60 parts by mass or less with respect to 100 parts by mass of the polymer.

5. The radiation-sensitive composition according to claim 1, wherein in the formula (2), W is a monocyclic lactone structure.

6. The radiation-sensitive composition according to claim 1, wherein in the formula (2), L is a single bond or a divalent alkanediyl group having 1 to 10 carbon atoms.

7. The radiation-sensitive composition according to claim 1, wherein a content ratio of the structural unit (I) in the polymer to all structural units constituting the polymer is 1 mol % or more and 70 mol % or less.

8. The radiation-sensitive composition according to claim 1, wherein the monovalent radiation-sensitive onium cation represented by Z⁺ is a sulfonium cation or an iodonium cation.

9. The radiation-sensitive composition according to claim 1, wherein the polymer comprises a plurality of structural units represented by the structural unit (I).

10. The radiation-sensitive composition according to claim 1, wherein the polymer further comprises a structural unit (II) which comprises an acid-dissociable group.

11. The radiation-sensitive composition according to claim 1, wherein the polymer further comprises a structural unit which comprises at least one selected from the group consisting of a polycyclic lactone structure, a polycyclic carbonate structure, and a polycyclic sultone structure.

12. The radiation-sensitive composition according to claim 1, further comprising an acid diffusion controlling agent.

13. A method for forming a pattern, the method comprising:
applying the radiation-sensitive composition according to claim 1 directly or indirectly to a substrate to form a resist film;
exposing the resist film to light; and
developing the exposed resist film with a developer.

14. The method for forming a pattern according to claim 13, wherein exposing comprises exposing the resist film to an ArF excimer laser or extreme ultraviolet rays.

15. An onium salt compound represented by formula (1-d), 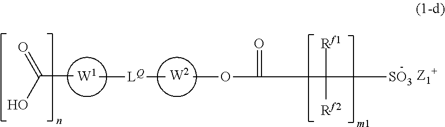
wherein in the formula (1-d),
W¹ and W² are each independently a substituted or unsubstituted cyclic structure,
L^{Q} is a single bond, *—OCO—, or *—O—CO—O—, * is a bond on the W¹ side,
R^{f1} and R^{f2} are each independently a hydrogen atom, a monovalent organic group having 1 to 20 carbon atoms, a fluorine atom, or a monovalent fluorinated hydrocarbon group, when there are a plurality of R^{f1}s and R^{f2}s, the plurality of R^{f1}s and R^{f2}s are the same or different from each other, at least one of R^{f1} and R^{f2} is a fluorine atom or a monovalent fluorinated hydrocarbon group,
ml is an integer of 1 to 4,
n is an integer of 1 to 3, and
Z₁⁺ represents a monovalent radiation-sensitive onium cation.
